(19) United States
(12) Patent Application Publication
(10) Pub. No.: US 2024/0132554 A1
(43) Pub. Date: Apr. 25, 2024

(54) METHOD OF TREATMENT OF MALARIA BY TARGETTING OPEN READING FRAMES

(71) Applicant: Cambridge Enterprise Limited, Cambridge (GB)

(72) Inventor: Sudhakaran PRABAKARAN, Acton, MA (US)

(21) Appl. No.: 18/274,839

(22) PCT Filed: Jan. 27, 2022
(86) PCT No.: PCT/IB2022/050732

§ 371 Date: Jul. 28, 2023

Related U.S. Application Data
(60) Provisional application No. 63/142,810, filed on Jan. 28, 2021.

(51) Int. Cl. C07K 14/445; C12N 15/113

(52) U.S. Cl. CPC C07K 14/445; C12N 15/113; C07K 2317/622; C12N 2310/14; C12N 2740/15043; C12N 2750/14143

(57) ABSTRACT

The present application features methods of diagnosing treating malaria by targeting novel open reading frames in Plasmodium falciparum. The novel open reading frames (nORFs) associated with malaria are expressed in a mosquito and in a human

### SEQUENCE LISTING

This application contains a Sequence Listing which has been filed electronically in ASCII format and is hereby incorporated by reference in its entirety. Said ASCII copy, created on Jan. 24, 2022, is named 51525-005WO2_Sequence_Listing_1_24_22_ST25.txt and is 98,339 bytes in size.

### BACKGROUND OF THE INVENTION

Plasmodium falciparum causes the deadliest form of malaria, which impacts over 200 million individuals and results in nearly 450,000 deaths each year, 60% of which are children aged under 5 years (WHO, 2018). It belongs to the large Apicomplexa phylum of diverse eukaryotic intracellular parasites. in addition to malaria, this phylum also encompasses infectious agents of cryptosporidiosis and toxoplasmosis. The latter is caused by Toxoplasma gondii, which is estimated to infect over 30% of the world population, and hence considered as one of the most successful parasites. These diseases are not only life-threatening but also widespread, and therefore present a serious threat to public health.

Unfortunately, both vaccines and treatment options with drugs are limited due to the biological complexity of the Apicomplexan parasites. Even for the well-studied plasmodium species, there is only one vaccine targeted for malaria with relatively low efficacy. The complexity arises from the highly regulated life cycles that allow them to inhabit different hosts and intracellular niches. Accordingly, new methods are needed for effective treatment of malaria and other related infectious diseases.

### SUMMARY OF THE INVENTION

In one aspect, the invention features a method of treating malaria in a subject. The method includes identifying a sequence of a novel open reading frame (nORF) in Plasmodium falciparum, wherein the sequence of the nORF is distinct from a canonical open reading frame (cORF) of a gene, wherein the nORF is present in (i) an overlapping region of the cORF in an alternate reading frame, (ii) a 5′ untranslated region (Urn) of the cORF, (iii) a 3′ UTR of the cORF, (iv) an intronic region of the cORF, (v) an intergenic region of the cORF, or (vi) a region not associated with the cORF or the gene, or in an antisense transcript thereof. The nORF may be expressed (e.g., above basal levels) in Plasmodium falciparum in a mosquito and in the subject. The method further includes administering to the subject an inhibitor that reduces expression of the nORF to treat malaria.

In another aspect, the invention features a method of treating malaria in a subject by administering to the subject an inhibitor that reduces expression of a nORF in Plasmodium falciparum. The subject may have previously been identified with a sequence of the nORF, wherein the sequence of the nORF is distinct from a cORF of a gene, wherein the nORF is present in (i) an overlapping region of the cORF in an alternate reading frame, (ii) a 5′ UTR of the cORF, (iii) a 3′ UTR of the cORF, (iv) an intronic region of the cORF, (v) an intergenic region of the cORF, or (vi) a region not associated with the cORF or the gene, or in an antisense transcript thereof. The nORF may he expressed (e.g., above basal levels) in Plasmodium falciparum in a mosquito in the subject.

In some embodiments, the nORF is expressed in an oocyst sporozoite (e.g., in a mosquito).

In some embodiments, the nORF is express in a salivary gland sporozoite (e.g., in the subject).

In some embodiments, the nORF is expressed in an oocyst sporozoite and in a salivary gland sporozoite.

In some embodiments, the nORF expression is increased in a salivary gland sporozoite (e.g. by at least 1%, 2%, 3%, 4%, 5%, 10%, 15%, 20%, 25%, 30%, 40%, 50%, 60%, 70%, 80%, 90%, 100%, 200%, 300%, 400%, 500%, or more), e.g., as compared to the basal nORF expression or as compared to the nORF expression in a different cell stage.

In some embodiments, the nORF expression is increased in an oocyst sporozoite (e.g. by at least 1%, 2%, 3%, 4%, 5%, 10%, 15%, 20%, 25%, 30%, 40%, 50%, 60%, 70%, 80%, 90%, 100%, 200%, 300%, 400%, 500%, or more), e.g., as compared to the basal nORF expression or as compared to the nORF expression in a different cell stage.

In some embodiments, the nORF expression is increased in an oocyst sporozoite (e.g. by at least 1%, 2%, 3%, 4%, 5%, 10%, 15%, 20%, 25%, 30%, 40%, 50%, 60%, 70%, 80%, 90%, 100%, 200%, 300%, 400%, 500%, or more), e.g., as compared to the basal nORF expression or as compared to the nORF expression in a different cell stage, and the nORF expression is increased in a salivary gland sporozoite (e.g. by at least 1%, 2%, 3%, 4%, 5%, 10%, 15%, 20%, 25%, 30%, 40%, 50%, 60%, 70%, 80%, 90%, 100%, 200%, 300%, 400%, 500%, or more), e.g., as compared to the basal nORF expression or as compared to the nORF expression in a different cell stage.

In some embodiments of either of the foregoing aspects, the method reduces expression of then nORF, e.g., by at least 5%, 10%, 15%, 20%, 25%, 30%, 40%, 50%, 60%, 70%, 80%, 90%, 95%, 97%, or 99%. The nORF may exhibit an increase (e.g. by at least 1%, 2%, 3%, 4%, 5%, 10%, 15%, 20%, 25%, 30%, 40%, 50%, 60%, 70%, 80%, 90%, 100%, 200%, 300%, 400%, 500%, or more) in expression, e.g., as compared to the basal nORF expression or as compared to the nORF expression in a different cell stage.

In some embodiments, the nORF is within an antisense transcript.

In some embodiments of either of the above aspects, the inhibitor is a small molecule, a polynucleotide, or a polypeptide. The polynucleotide may include, e.g., a miRNA, an antisense RNA, an shRNA, or an siRNA. The polypeptide may include, e.g., an antibody or antigen-binding fragment thereof (e.g., an scFv).

In some embodiments, the inhibitor is encoded by a vector, such as a viral vector. The viral vector may be selected, for example, from the group consisting of a Retroviridae family virus, an adenovirus, a parvovirus, a coronavirus, a rhabdovirus, a paramyxovirus, a picornavirus, an alphavirus, a herpes virus, and a poxvirus. The parvovirus viral vector may be, for example, an adeno-associated virus (AAV) vector.

In some embodiments, the viral vector is a Retroviridae family viral vector (e.g., a ientiviral vector, an alpharetroviral vector, or a gammaretroviral vector). The Retroviridae family viral vector may include, e.g., one or more of the following: a central polypurine tract, a woodchuck hepatitis virus post-transcriptional regulatory element, a 5′-LTR, HIV signal sequence, HIV Psi signal 5′-splice site, delta-GAG element, 3′-splice site, and a 3′-self inactivating LTR.

In some embodiments, the viral vector is a pseudotyped viral vector. The pseudotyped viral vector may be selected, for example, from the group consisting of a pseudotyped adenovirus, a pseudotyped parvovirus, a pseudotyped coronavirus, a pseudotyped rhabdovirus, a pseudotyped paramyxovirus, a pseudotyped picornavirus, a pseudotyped alphavirus, a pseudotyped herpes virus, a pseudotyped poxvirus, and a pseudotyped Retroviridae family virus. The pseudotyped viral vector may be, e.g., a lentiviral vector.

In some embodiments, the pseudotyped viral vector includes one or more envelope proteins from a virus selected from vesicular stomatitis virus (VSV), RD114 virus, murine leukemia virus (MLV), feline leukemia virus (FeLV), Venezuelan equine encephalitis virus (VEE), human foamy virus (HFV), walleye dermal sarcoma virus (WDSV), Semliki Forest virus (SFV), Rabies virus, avian leukosis virus (ALV), bovine immunodeficiency virus (BIV), bovine leukemia virus (BLV), Epstein-Barr virus (EBV), Caprine arthritis encephalitis virus (CAEV), Sin Nombre virus (SNV), Cherry Twisted Leaf virus (ChTLV), Simian T-cell leukemia virus (STLV). Mason-Pfizer monkey virus (MPMV), squirrel monkey retrovirus (SMRV), Rous-associated virus (RAV), Fujinami sarcoma virus (FuSV), avian carcinoma virus (MH2), avian encephalomyelitis virus (AEV), Alfa mosaic virus (AMV), avian sarcoma virus CT10, and equine infectious anemia virus (EIAV).

In some embodiments, the pseudotyped viral vector includes a VSV-G envelope protein,

In some embodiments, the encoded protein product of the nORF is less than about 100 amino acids (e.g., less than about 95, 90, 85, 80, 75, 70, 65, 60, 55, or 50 amino acids).

In some embodiments, the method further comprises performing a statistical analysis between the expression of nORF in the mosquito and expression of the nORF in the subject.

In some embodiments, the statistical analysis measures a positive or negative association between the expression of the nORF in the mosquito and expression of the nORF in the subject.

In some embodiments, the nORF has at least 85% (e.g., at least 90%, 95%, 97%, 98%, 99%, or 100%) sequence identity to any one of SEQ NOs: 1-13 or a portion thereof.

In some embodiments, the gene product of the nORF has at least 85% (e.g., at least 90%, 95%, 97%, 98%, 99%, or 100%) sequence identity to any one of SEQ 1E) NOs: 14-28,

In some embodiments, the nORF has at least 85% (e.g., at least 90%, 95%, 97%, 98%, 99%, or 100%) to SEQ ID NO: 1 or a portion thereof. in some embodiments, the nORF has at least 85% (e.g., at least 90%, 95%, 97%, 98%, 99%, or 100%) to SEQ ID NO: 2 or a portion thereof. In some embodiments, the nORF has at least 85% (e.g., at least 90%, 95%, 97%, 98%, 99%, or 100%) to SEQ ID NO: 3 or a portion thereof. In some embodiments, the nORF has at least 85% ((e.g., at least 90%, 95%, 97%, 98%, 99%, or 100%) to SEQ ID NO: 4 or a portion thereof. In some embodiments, the nORF has at least 85% (e.g., at least 90%, 95%, 97%, 98%, 99%, or 100%) to SEQ ID NO: 5 or a portion thereof. In some embodiments, the nORF has at least 85% (e.g., at least 90%, 95%, 97%, 98%, 99%, or 100%) to SEQ ID NO: 6 or a portion thereof. In some embodiments, the nORF has at least 85% (e.g., at least 90%, 95%, 97%, 98%, 99%, or 100%) to SEQ ID NO: 7 or a portion thereof. In some embodiments, the nORF has at least 85% (e.g., at least 90%, 95%, 97%, 98%, 99%, or 100%) to SEQ ID NO: 8 or a portion thereof. In some embodiments, the nORF has at least 85% (e.g., at least 90%, 95%, 97%, 98%, 99%, or 100%) to SEQ ID NO: 9 or a portion thereof. In some embodiments, the nORF has at least 85% (e.g., at least 90%, 95%, 97%, 98%, 99%, or 100%) to SEQ ID NO: 10 or a portion thereof. In some embodiments, the nORF has at least 85% (e.g., at least 90%, 95%, 97%, 98%, 99%, or 100%) to SEQ ID NO: 11 or a portion thereof. in some embodiments, the nORF has at least 85% (e.g., at least 90%, 95%, 97%, 98%, 99%, or 100%) to SEQ ID NO: 12 or a portion thereof. In some embodiments, the nORF has at least 85% (e.g., at least 90%, 95%, 97%, 98%, 99%, or 100%) to SEQ ID NO: 13 or a portion thereof.

In some embodiments, the gene product of the nORF has at least 85% (e.g., at least 90%, 95%, 97%, 98%, 99%, or 100%) sequence identity to SEQ 10 NO: 14. In some embodiments, the gene product of the nORF has at least 85% (e.g., at least 90%, 95%, 97%, 98%, 99%, or 100%) sequence identity to SEQ ID NO: 15. In some embodiments, the gene product of the nORF has at least 85% (e.g., at least 90%, 95%, 97%, 98%, 99%, or 100%) sequence identity to SEQ ID NO: 16. In some embodiments, the gene product of the nORF has at least 85% (e.g., at least 90%, 95%, 97%, 98%, 99%, or 100%) sequence identity to SEQ ID NO: 17. In some embodiments, the gene product of the nORF has at least 85% (e.g., at least 90%, 95%, 97%, 98%, 99%, or 100%) sequence identity to SEQ ID NO: 18. In some embodiments, the gene product of the nORF has at least 85% (e.g., at least 90%, 95%, 97%, 98%, 99%, or 100%) sequence identity to SEQ ID NO: 19. In some embodiments, the gene product of the nORF has at least 85% (e.g., at least 90%, 95%, 97%, 98%, 99%, or 100%) sequence identity to SEQ ID NO: 20. In some embodiments, the gene product of the nORF has at least 85% (e.g., at least 90%, 95%, 97%, 98%, 99%, or 100%) sequence identity to SEQ ID NO: 21. In some embodiments, the gene product of the nORF has at least 85% (e.g., at least 90%, 95%, 97%, 98%, 99%, or 100%) sequence identity to SEQ ID NO: 22. In some embodiments, the gene product of the nORF has at least 85% (e.g., at least 90%, 95%, 97%, 98%, 99%, or 100%) sequence identity to SEQ ID NO: 23. In some embodiments, the gene product of the nORF has at least 85% (e.g., at least 90%, 95%, 97%, 98%, 99%, or 100%) sequence identity to SEQ ID NO: 24, In some embodiments, the gene product of the nORF has at least 85% (e.g., at least 90%, 95%, 97%, 98%, 99%, or 100%) sequence identity to SEQ ID NO: 25. In some embodiments, the gene product of the nORF has at least 85% (e.g., at least 90%, 95%, 97%, 98%, 99%, or 100%) sequence identity to SEQ ID NO: 26. In some embodiments, the gene product of the nORF has at least 85% (e.g., at least 90%, 95%, 97%, 98%, 99%, or 100%) sequence identity to SEQ ID NO: 27. In some embodiments, the gene product of the nORF has at least 85% (e.g., at least 90%, 95%, 97%, 98%, 99%, or 100%) sequence identity to SEQ ID NO: 28.

In some embodiments, the method includes diagnosing the subject as having malaria and then treating the subject for malaria.

### Definitions

As used herein, “expressed” refers to transcription and/or translation of an open reading frame at least 1%, 2%, 3%, 4%, 5%, 6%, 7%, 8%, 9%, 10%, 15%, 20%, 25%, 30%, 40%, 50%, 60%, 70%, 80%, 90%, 100%, or more above a basal level of transcription and/or translation, Basal expression may be caused by leaky expression of an open reading frame as would be understood by one of skill in the art.

As used herein, a “novel open reading frame” or “nORF” refers to an open reading frame that is transcribed in a cell and consists of a sequence that is distinct from a canonical open reading frame (cORF) transcribed from a gene. The nORF may be present in (i) an overlapping region of the cORF in an alternate reading frame, (ii) a 5′ untranslated region (UTR) of the cORF, (iii) a 3′ UTR of the cORF, (iv) an intronic region of the cORF, (v) an intergenic region of the cORF, or (vi) a region not associated with a cORF or the gene. The nORF may be any unannotated genetic sequence that is transcribed in a cell. The nORF may be present in an antisense transcript.

As used herein, a “canonical open reading frame” or “cORF” refers to an open reading frame that is transcribed in a cell and its associated genetic elements, including the 5′ UTR, the 3′ UTR, the intronic regions, the exonic regions, and the intergenic regions flanking the gene comprising the cORF. A cORF includes either the primary open reading frame that is expressed from a gene, the most abundantly expressed open reading frame expressed from a gene, or an ORF that is annotated in a publicly available database as the primary and/or most abundantly expressed open reading frame from a gene.

### BRIEF DESCRIPTION OF THE DRAWINGS

FIGS. 1A and 1B are schematic diagrams showing the location of a nORF (FIG. 1A) and a proteogenomic workflow (FIG. 1B) to identify translational products from novel open reading frames using transcriptomes and proteomes. oo-spz: occyst sporozoite, sg-spz: salivary gland sporozoite

FIGS. 2A and 2B are a set of graphs showing distribution of number of unique peptides mapped to novel transcripts identified in occyst sporozoite (FIG. 2A) and salivary gland sporozoite (FIG. 2B).

FIG. 3A-3C are a set of graphs showing distribution of nORF categories in total proteomes of oocyst sporozoite (FIG. 3A) and salivary gland sporozoite (FIG. 3B), as well as in differentially expressed nORFs (FIG. 3C).

FIGS. 4A and 4B are graphs showing gene ontology (GO) terms (FIG. 4A) and Kyoto encyclopedia of genes and genomes (KEGG) pathways (FIG. 4B) enrichment of the reference genes associated with novel peptides identified in cocyst sporozoite and salivary gland sporozoite. For GO terms enrichment the p-value cut-off was 0.01, and 0.02 for KEGG pathways. Percentage of genes means how many percent of the background genes (in Plasmodium falciparum) with a particular term are present in the gene set of interest.

FIGS. 5A-5C are drawings showing predicted structures of differentially expressed nORF with 3′UTR (FIG. 5A), differentially expressed intergenic nORF (FIG. 5B), and two AltORFs (FIG. 5C) that have protein expression that was anticorrelated with the associated reference genes.

FIGS. 6A and 6B are graphs showing predicted protein domains in high quality (Mascot score>20) intergenic ORFs (FIG. 6A; total ORFs=32) and AltORFs (FIG. 6B; total ORFs=248).

FIG. 7 is a schematic drawing of the different developmental stages in P. falciparum involving mosquito vector and human host (Adapted from “DNA Repair Mechanisms and Their Biological Roles in the Malaria Parasite Plasmodium falciparum” by Lee et al., 2014 with modifications)

FIG. 8 is a flow chart showing the concept of proteogenomic approach where genomics, transcriptomics, and proteomics are integrated to improve genome annotation and gene models (LC-MS/MS: Liquid Chromatography with tandem mass spectrometry).

FIG. 9 is a schematic drawing show definitions of novel open reading frames with respect to the structure of a canonical protein coding gene (exons).

FIGS. 10A and 10B are schematic drawings showing estimation of lull-length ORF from peptides matched to a translated transcript, where M is methionine translated from the start codon and * is translated from stop codon. FIG. 10A shows a schematic drawing inside ORF and FIG. 10B shows a schematic drawing outside ORF.

FIGS. 11A and 11B are graphs showing correlation of mRNA log fold change (FIG. 11A) and spectral abundance factors (SAF) (FIG. 11B) between the reference values for canonical genes in an oocyst sporozoite (FIG. 11B; left panel) and in a salivary gland sporozoite (FIG. 11B; right panel).

FIG. 12 is a schematic diagram showing the RNA-seq coverage of the 3′UTR of PF3D7_1013400 and the transmembrane region prediction by TMHMM. The peptide-spectrum match that maps to the 3′UTR was only found in oocyst sporozoite. oo-spz: oocyst sporozoite, sg-spz: salivary gland sporozoite.

### DETAILED DESCRIPTION

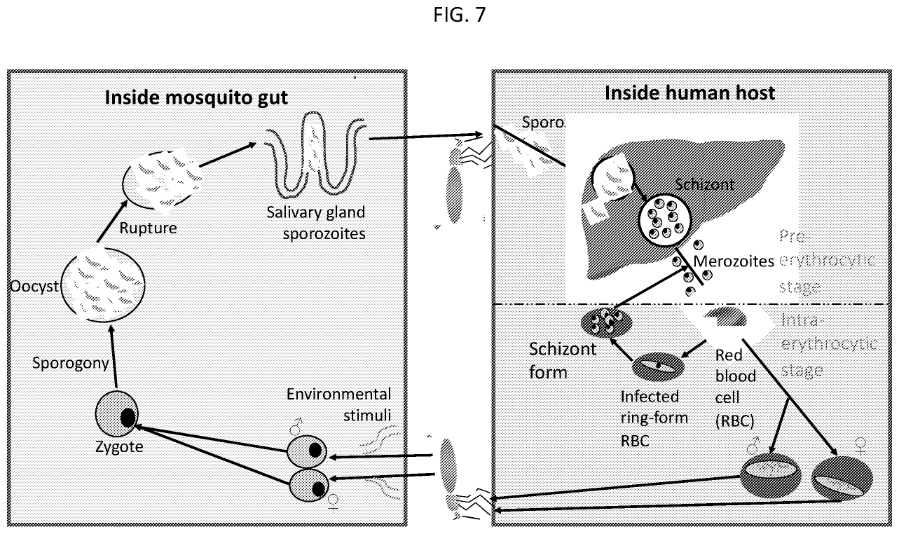

Described herein are methods of diagnosing and treating malaria. Malaria is caused by infection of Plasmodium falciparum, a unicellular protozoan. P. falciparum can infect two separate hosts, the Anopheles mosquito and a human. The developmental cycle starts with the malaria infected mosquito taking a blood meal from a human where it transmits sporozoites of plasmodium parasites from the mosquito salivary gland to the human host (FIG. 7). The sporozoites then travel to the liver and infect liver cells, allowing them to replicate and mature into schizonts. They then rupture and release merozoites, which infect red blood cells and is the stage that causes clinical manifestation of malaria. Some of the blood-stage parasites mature into a schizont, an asexual multiplying form that ruptures and releases more merozoites to infect red blood cells. They can also develop into sexual precursor cells, known as gametocytes, which are ingested by the Anopheles mosquito during a blood meal. The gametocytes are activated by environmental stimuli inside mosquito midgut and differentiate into gametes and fuse to form a zygote. The zygotes further develop into oocysts, within which sporogony takes place and produces sporozoites to invade mosquito's salivary gland, ready for next round of infection via a mosquito bite.

The present invention is premised, in part, upon the discovery that certain novel open reading frames (nORFs)—short, unannotated, expressed gene products—are differentially expressed during different developmental stages in P. falciparum. For example, the progression from one developmental stage to another involves drastic changes in gene expression, and infectivity of the parasites to human hosts can vary depending on the stage. For instance, while oocyst sporozoites are highly infectious for the mosquito salivary gland, they are non-infectious to mammalian hosts; on the contrary, salivary gland sporozoites exhibit specific infectivity for mammalian liver, but correspondingly loose infectivity for mosquito's salivary gland.

The present invention features methods of treating malaria by targeting one or more of these nORFs to reduce or eliminate infectivity by the parasite. The methods of treatment are described in more detail below.

### Methods of Diagnosis

Genetic testing offers one avenue by which a patient may be diagnosed as having or is at risk of developing malaria. For example, a genetic analysis can he used to determine whether a patient has a malaria infection by identifying expression of a nORF as described herein. The nORF may be present in any region of a P. falciparum gene, such as within the cORF, a 5′ untranslated region (UTR) of the cORF, a 3′ UTR of the cORF, an intronic region of the cORF, or an intergenic region of the cORF, or in an antisense transcript thereof. The nORF may be present in a region that is not associated with the cORF or the gene.

Exemplary genetic tests that can be used to determine whether a patient has expression of the nORF include polymerase chain reaction (PCR) methods known in the art, such as DNA and RNA sequencing. In some embodiments, the subject is identified as having expression of a P. falciparum associated gene, which may be annotated in a publicly available database as being associated with malaria. nORF sequences may be identified de novo, e.g., using computational or statistical methods. Furthermore, nORF sequences may be identified from publicly available databases in genomic sequences in which the nORF was not previously identified and/or annotated as a sequence that was expressed and/or translated.

### Methods of Treatment

The invention features methods of treating malaria by targeting a nORF that is expressed in Plasmodium. The nORF may be expressed in an oocyst sporozoite (e.g., in a mosquito). The nORF may be expressed in a salivary gland sporozoite (e.g., in the subject). In some embodiments, the nORF is expressed in an oocyst sporozoite and in a salivary gland sporozoite.

In some embodiments, the nORF has differential expression (e.g., increased or decreased expression) in one cell stage (e.g., oocyst sporozoite, e.g., in a mosquito) versus another (e.g., salivary gland sporozoite, e.g., in a human) or vice versa. The nORF may exhibit an increase (e.g. by at least 1%, 2%, 3%, 4%, 5%, 10%, 15%, 20%, 25%, 30%, 40%, 50%, 60%, 70%, 80%, 90%, 100%, 200%, 300%, 400%, 500%, or more) in expression, e.g., as compared to basal expression, or as compared to nORF expression in a different cell stage. In some embodiments, the nORF may exhibit an increase in expression (e.g. by at least 5%, 10%, 15%, 20%, 25%, 30%, 40%, 50%, 60%, 70%, 80%, 90%, 100%, 200%, 300%, 400%, 500%, or more), e.g., in two cell stages, e.g., in the oocyst sporozoite and in the salivary gland sporozoite.

The subject may be first determined to have the expressed nORF and then may be subsequently be treated for malaria. The subject may have previously been determined to have the expressed nORF and is then treated for malaria, The treatment varies according to the expressed nORF associated with the disease. For example, the treatment may include an inhibitor that targets the nORF to decrease (e.g., by at least 5%, 10%, 15%, 20%, 25%, 30%, 40%, 50%, 60%, 70%, 80%, 90%, 95%, 97%, or 99%) expression of the nORF.

In some embodiments, the length of the nORF is less than about 100 amino acids (e.g., from about 50 to 100, 50 to 90, 50 to 80, 60 to 90, 60 to 80, 70 to 100, 70 to 90, 70 to 80. 80 to 100, or 90 to 100 amino acids).

In some embodiments, the nORF is within an antisense transcript.

In some embodiments, the nORF has at least 85% (e.g., at least 90%, 95%, 97%, 98%, 99%, or 100%) sequence identity to any one of SEQ ID NOs:1 -13 or a portion thereof.

In some embodiments, the gene product of the nORF has at least 85% (e.g., at least 90%, 95%, 97%, 98%, 99%, or 100%) sequence identity to any one of SEQ ID NOs: 14-28.

In some embodiments, the nORF has at least 85% (e.g., at least 90%, 95%, 97%, 98%, 99%, or 100%) to SEQ ID NO: 1 or a portion thereof. in some embodiments, the nORF has at least 85% (e.g., at least 90%, 95%, 97%, 98%, 99%, or 100%) to SEQ ID NO: 2 or a portion thereof. In some embodiments, the nORF has at least 85% (e.g., at least 90%, 95%, 97%, 98%, 99%, or 100%) to SEQ ID NO: 3 or a portion thereof. In some embodiments, the nORF has at least 85% (e.g., at least 90%, 95%, 97%, 98%, 99%, or 100%) to SEQ ID NO: 4 or a portion thereof. In some embodiments, the nORF has at least 85% (e.g., at least 90%, 95%, 97%, 98%, 99%, or 100%) to SEQ ID NO: 5 or a portion thereof. In some embodiments, the nORF has at least 85% (e.g., at least 90%, 95%, 97%, 98%, 99%, or 100%) to SEQ ID NO: 6 or a portion thereof. In some embodiments, the nORF has at least 85% (e.g., at least 90%. 95%, 97%, 98%, 99%, or 100%) to SEQ ID NO: 7 or a portion thereof. In some embodiments, the nORF has at least 85% (e.g., at least 90%, 95%, 97%, 98%, 99%, or 100%) to SEQ ID NO: 8 or a portion thereof. In some embodiments, the nORF has at least 85% (e.g., at least 90%, 95%, 97%, 98%, 99%, or 100%) to SEQ ID NO: 9 or a portion thereof. In some embodiments, the nORF has at least 85% (e.g., at least 90%, 95%, 97%, 98%, 99%, or 100%) to SEQ ID NO: 10 or a portion thereof. In some embodiments, the nORF has at least 85% (e.g., at least 90%, 95%, 97%, 98%, 99%, or 100%) to SEQ ID NO: 11 or a portion thereof. In some embodiments, the nORF has at least 85% (e.g., at least 90%, 95%, 97%, 98%, 99%, or 100%) to SEQ ID NO: 12 or a portion thereof. In some embodiments, the nORF has at least 85% (e.g., at least 90%, 95%, 97%, 98%, 99%, or 100%) to SEQ ID NO: 13 or a portion thereof.

In some embodiments, the gene product of the nORF has at least 85% (e.g., at least 90%, 95%, 97%, 98%, 99%, or 100%) sequence identity to SEQ ID NO: 14. In some embodiments, the gene product of the nORF has at least 85% (e.g., at least 90%, 95%, 97%, 98%, 99%, or 100%) sequence identity to SEQ ID NO: 15. In some embodiments, the gene product of the nORF has at least 85% (e.g., at least 90%, 95%, 97%, 98%, 99%, or 100%) sequence identity to SEQ ID NO: 16. In some embodiments, the gene product of the nORF has at least 85% (e.g., at least 90%, 95%, 97%, 98%, 99%, or 100%) sequence identity to SEQ ID NO: 17. In some embodiments, the gene product of the nORF has at least 85% (e.g., at least 90%, 95%, 97%, 98%, 99%, or 100%) sequence identity to SEQ ID NO: 18. in some embodiments, the gene product of the nORF has at least 85% (e.g., at least 90%, 95%, 97%, 98%, 99%, or 100%) sequence identity to SEQ ID NO: 19. In some embodiments, the gene product of the nORF has at least 85% (e.g., at least 90%, 95%, 97%. 98%, 99%, or 100%) sequence identity to SEQ ID NO: 20. In some embodiments, the gene product of the nORF has at least 85% (e.g., at least 90%, 95%, 97%, 98%, 99%, or 100%) sequence identity to SEQ ID NO: 21. In some embodiments, the gene product of the nORF has at least 85% (e.g., at least 90%, 95%, 97%, 98%, 99%, or 100%) sequence identity to SEQ ID NO: 22. In some embodiments, the gene product of the nORF has at least 85% (e.g., at least 90%, 95%, 97%, 98%, 99%, or 100%) sequence identity to SEQ ID NO: 23. In some embodiments, the gene product of the nORF has at least 85% (e.g., at least 90%, 95%, 97%, 98%, 99%, or 100%) sequence identity to SEQ ID NO: 24. In some embodiments, the gene product of the nORF has at least 85% (e.g., at least 90%, 95%, 97%, 98%, 99%, or 100%) sequence identity to SEQ ID NO: 25. In some embodiments, the gene product of the nORF has at least 85% (e.g., at least 90%, 95%, 97%, 98%, 99%, or 100%) sequence identity to SEQ ID NO: 26. In some embodiments, the gene product of the nORF has at least 85% (e.g., at least 90%, 95%, 97%, 98%, 99%, or 100%) sequence identity to SEQ ID NO: 27. In some embodiments, the gene product of the nORF has at least 85% (e.g., at least 90%, 95%, 97%, 98%, 99%, or 100%) sequence identity to SEQ ID NO: 28.

### Inhibitors

The methods of treatment described herein may include providing an inhibitor that targets the expressed nORF. The inhibitor may reduce (e.g., by at least 5%, 10%, 15%, 20%, 25%, 30%, 40%, 50%, 60%, 70%, 80%, 90%, 95%, 97%, or 99%) an amount or activity of the expressed nORF, such as to prevent the deleterious effect of the expressed nORF. The inhibitor may target the polynucleotide containing the nORF or the protein encoded by the nORF. The inhibitor may be a small molecule, a polynucleotide, or a polypeptide. Suitable small molecules may be determined or identified by using computational analysis based on the structure of the nORF as determined by a protein bolding algorithm. The small molecule may target any region of the nORF. The small molecule may target the nORF or the protein encoded by the nORF. Suitable polypeptides for reducing an activity or amount of the nORF include, for example, an antibody or antigen-binding fragment thereof that binds to the nORF (e.g., a single chain antibody or antigen-binding fragment thereof). Suitable polynucleotides that can reduce an amount or activity of the nORF include RNA. For example, an RNA for reducing an activity or amount of the nORF may be, for example, a miRNA, an antisense RNA, an shRNA, or an siRNA. The miRNA, antisense RNA, shRNA, or siRNA may target a region of RNA (e.g., nORF gene) to reduce expression of the nORF. The polynucleotide may be an aptamer, e.g., an RNA aptamer that binds to and/or reduces an amount and/or activity of the nORF or the protein encoded by the nORF. The inhibitor may be provided directly or may be provided by a vector (e.g., a viral vector) encoding the inhibitor. The inhibitor may be formulated, e.g, in a pharmaceutical composition containing a pharmaceutically acceptable carrier. The composition can be administered by any suitable method known in the art to the skilled artisan. The composition (e.g., a vector, e.g., a viral vector) may be formulated in a virus or a virus-like particle,

### Nucleic Acid Mediated Knockdown

Using the compositions and methods described herein, a patient with malaria may be administered an interfering RNA molecule, a composition containing the same, or a vector encoding the same, so as to reduce or suppress the expression of a nORF. Exemplary interfering RNA molecules that may be used in conjunction with the compositions and methods described herein are siRNA molecules, miRNA molecules, shRNA molecules, and antisense RNA molecules, among others. In the case of siRNA molecules, the siRNA may be single stranded or double stranded. miRNA molecules, in contrast, are single-stranded molecules that form a hairpin, thereby adopting a hydrogen-bonded structure reminiscent of a nucleic acid duplex. In either case, the interfering RNA may contain an antisense or “guide” strand that anneals (e.g., by way of complementarity) to the repeat-expanded mutant RNA target. The interfering RNA may also contain a “passenger” strand that is complementary to the guide strand and, thus, may have the same nucleic acid sequence as the RNA target.

siRNA is a class of short (e.g., 20-25 nt) double-stranded non-coding RNA that operates within the RNA interference pathway. siRNA may interfere with expression of the nORF gene with complementary nucleotide sequences by degrading mRNA (via the Dicer and RISC pathways) after transcription, thereby preventing translation. miRNA is another short (e.g., about 22 nucleotides) non-coding RNA molecule that functions in RNA silencing and post-transcriptional regulation of gene expression. miRNAs function via base-pairing with complementary sequences within mRNA molecules, thereby leading to cleavage of the mRNA strand into two pieces and destabilization of the mRNA through shortening of its poly(A) tail. shRNA is an artificial RNA molecule with a tight hairpin turn that can be used to silence target gene expression via RNA interference. Antisense RNA are also short single stranded molecules that hybridize to a target RNA and prevent translation by occluding the translation machinery, thereby reducing expression of the target (e.g., the nORF).

### Antibody Mediated Knockdown

Using the compositions and methods described herein, a patient with malaria may be provided an antibody or antigen-binding fragment thereof, a composition containing the same, a vector encoding the same, or a composition of cells containing a vector encoding the same, so as to suppress or reduce the activity of the expressed nORF. In some embodiments of the compositions and methods described herein, an antibody or antigen-biding fragment thereof may be used that binds to and reduces or eliminates the activity of the nORF. The antibody may be monoclonal or polyclonal. In some embodiments, the antigen-binding fragment is an antibody that lacks the Fc portion, an F(ab′)2, a Fab, an Fv, or an scFv. The antigen-binding fragment may be an scFv.

One of ordinary skill in the art will appreciate that an antibody may include four polypeptides: two identical copies of a heavy chain polypeptide and two copies of a light chain polypeptide. Each of the heavy chains contains one N-terminal variable (V_{H}) region and three C-terminal constant (CH1, CH2 and CH3) regions, and each light chain contains one N-terminal variable (V_{L}) region and one C-terminal constant (C_{L}) region. Thus, one of skill in the art would appreciate that as described herein, a vector that includes a transgene that encodes a polypeptide that is an antibody may be a single transgene that encodes a plurality of polypeptides. Also contemplated is a vector that includes a plurality of transgenes, each transgene encoding a separate polypeptide of the antibody. All variations are contemplated herein. The variable regions of each pair of light and heavy chains form the antigen binding site of an antibody. The transgene which encodes an antibody directed against the nORF can include one or more transgene sequences, each of which encodes one or more of the heavy and/or light chain polypeptides of an antibody. In this respect, the transgene sequence which encodes an antibody directed against the nORF can include a single transgene sequence that encodes the two heavy chain polypeptides and the two light chain polypeptides of an antibody. Alternatively, the transgene sequence which encodes an antibody directed against the nORF can include a first transgene sequence that encodes both heavy chain polypeptides of an antibody, and a second transgene sequence that encodes both light chain polypeptides of an antibody. In yet another embodiment, the transgene sequence which encodes an antibody can include a first transgene sequence encoding a first heavy chain polypeptide of an antibody, a second transgene sequence encoding a second heavy chain polypeptide of an antibody, a third transgene sequence encoding a first light chain polypeptide of an antibody, and a fourth transgene sequence encoding a second light chain polypeptide of an antibody.

In some embodiments, the transgene that encodes the antibody includes a single open reading frame encoding a heavy chain and a light chain, and each chain is separated by a protease cleavage site.

In some embodiments, the transgene encodes a single open reading frame encoding both heavy chains and both light chains, and each chain is separate by protease cleavage site.

In some embodiments, full-length antibody expression can be achieved from a single transgene cassette using 2A peptides, such as foot-and-mouth disease virus (FMDV) equine rhinitis A, porcine teschovirus-1, and Thosea asigna virus 2A peptides, which are used to link two or more genes and allow the translated polypeptide to be self-cleaved into individual polypeptide chains (e.g., heavy chain and light chain, or two heavy chains and two light chains). Thus, in some embodiments, the transgene encodes a 2A peptide in between the heavy and light chains, optionally with a flexible linker flanking the 2A peptide (e.g., GSG linker). The transgene may further include one or more engineered cleavage sequences, e.g., a furin cleavage sequence to remove the 2A peptide residues attached to the heavy chain or light chain. Exemplary 2A peptides are described, e.g., in Chng et al MAbs 7: 403-412, 201f5, and Lin et al, Front, Plant Sol. 9:1379, 2018, the disclosures of which are hereby incorporated by reference in their entirety.

In some embodiments, the antibody is a single-chain antibody or antigen-binding fragment thereof expressed from a single transgene.

### Viral Vectors for Expression

Viral genomes provide a rich source of vectors that can be used for the efficient delivery of exogenous genes into a mammalian cell, The gene to be delivered may include an inhibitor that targets a nORF, such as an RNA (e.g., an aptamer, a miRNA, an antisense RNA, an shRNA, or an siRNA). Viral genomes are particularly useful vectors for gene delivery as the polynucleotides contained within such genomes are typically incorporated into the nuclear genome of a mammalian cell by generalized or specialized transduction. These processes occur as part of the natural viral replication cycle, and do riot require added proteins or reagents in order to induce gene integration. Examples of viral vectors are a retrovirus (e.g., Retroviridae family viral vector), adenovirus (e.g., Ad5, Ad26, Ad34, Ad35, and Ad48), parvovirus (e.g., an adeno-associated viral (AAV) vector), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus), and poxvirus (e.g., vaccinia, modified vaccinia Ankara (MVA), fowlpox and canarypox). Other viruses include Norwalk virus, togavirus, flavivirus, reoviruses, papovavirus, hepadnavirus, human papilloma virus, human foamy virus, and hepatitis virus, for example. Examples of retroviruses are: avian leukosis-sarcoma, avian C-type viruses, mammalian C-type, B-type viruses, D-type viruses, oncoretroviruses, HTLV-BLV group, lentivirus, alpharetrovirus, gammaretrovirus, spumavirus (Coffin, J. M., Retroviridae: The viruses and their replication, Virology, Third Edition (Lippincott-Raven, Philadelphia, (1996))). Other examples are murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, in McVey et al., (U.S. Pat. No. 5,801,030), the teachings of which are incorporated herein by reference.

### Retro Viral Vectors

The delivery vector used in the methods described herein may be a retroviral vector, One type of retroviral vector that may be used in the methods and compositions described herein is a lentiviral vector. Lentivral vectors (LVs), a subset of retroviruses, transduce a wide range of dividing and non-dividing cell types with high efficiency, conferring stable, long-term expression of the transgene encoding the polypeptide or RNA. An overview of optimization strategies for packaging and transducing LVs is provided in Delenda, The Journal of Gene Medicine 6: S125 (2004), the disclosure of which is incorporated herein by reference.

The use of lentivirus-based gene transfer techniques relies on the in vitro production of recombinant lentivirai particles carrying a highly deleted viral genome in which the agent of interest is accommodated. In particular, the recombinant lentivirus are recovered through the in trans coexpression in a permissive cell line of (1) the packaging constructs, i.e., a vector expressing the Gag-Pol precursors together with Rev (alternatively expressed in trans); (2) a vector expressing an envelope receptor, generally of an heterologous nature; and (3) the transfer vector, consisting in the viral cDNA deprived of all open reading frames, but maintaining the sequences required for replication, encapsiciation, and expression, in which the sequences to be expressed are inserted.

A LV used in the methods and compositions described herein may include one or more of a 5′-Long terminal repeat (LTR), HIV signal sequence, HIV Psi signal 5-splice site (SD), delta-GAG element, Rev Responsive Element (RRE), 3′-splice site (SA), elongation factor (EF) 1-alpha promoter and 3′-self inactivating LTR (SIN-LTR), The lentiviral vector optionally includes a central polypurine tract (cPPT) and a woodchuck hepatitis virus post-transcriptional regulatory element (WPRE), as described in U.S. Pat. No. 6,136,597, the disclosure of which is incorporated herein by reference as it pertains to WPRE. The lentiviral vector may further include a pHR′ backbone, which may include for example as provided below.

The Lentigen LV described in Lu et al., Journal of Gene Medicine 6:963 (2004) may be used to express the DNA molecules and/or transduce cells. A LV used in the methods and compositions described herein may a 5′-Long terminal repeat (LTR), HIV signal sequence, HIV Psi signal 5′-splice site (SD), delta-GAG element, Rev Responsive Element (RRE), 3′-splice site (SA), elongation factor (EF) 1-alpha promoter and 3′-self inactivating L TR (SIN-LTR). It will be readily apparent to one skilled in the art that optionally one or more of these regions is substituted with another region performing a similar function.

Enhancer elements can be used to increase expression of modified DNA molecules or increase the lentiviral integration efficiency. The LV used in the methods and compositions described herein may include a nef sequence. The LV used in the methods and compositions described herein may include a cPPT sequence which enhances vector integration. The cPPT acts as a second origin of the (+)-strand DNA synthesis and introduces a partial strand overlap in the middle of its native HIV genome. The introduction of the cPPT sequence in the transfer vector backbone strongly increased the nuclear transport and the total amount of genome integrated into the DNA of target cells. The LV used in the methods and compositions described herein may include a Woodchuck Posttranscriptional Regulatory Element (WPRE). The WPRE acts at the transcriptional level, by promoting nuclear export of transcripts and/or by increasing the efficiency of polyadenylation of the nascent transcript, thus increasing the total amount of mRNA in the cells. The addition of the WPRE to LV results in a substantial improvement in the level of expression from several different promoters, both in vitro and in vivo. The LV used in the methods and compositions described herein may include both a cPPT sequence and WPRE sequence. The vector may also include an IRES sequence that permits the expression of multiple polypeptides from a single promoter.

In addition to IRES sequences, other elements which permit expression of multiple polypeptides are useful. The vector used in the methods and compositions described herein may include multiple promoters that permit expression more than one polypeptide, The vector used in the methods and compositions described herein may include a protein cleavage site that allows expression of more than one polypeptide. Examples of protein cleavage sites that allow expression of more than one polypeptide are described in Klump et al., Gene Ther.; 8:811 (2001), Osborn et al., Molecular Therapy 12:569 (2005), Szymczak and Vignali, Expert Opin Biol Ther. 5:627 (2005), and Szymczak et al., Nat Biotechnol. 22:589 (2004), the disclosures of which are incorporated herein by reference as they pertain to protein cleavage sites that allow expression of more than one polypeptide. it will be readily apparent to one skilled in the art that other elements that permit expression of multiple polypeptides identified in the future are useful and may be utilized in the vectors suitable for use with the compositions and methods described herein.

The vector used in the methods and compositions described herein may, he a clinical grade vector.

The viral vectors (e.g., retroviral vectors, e.g., lentiviral vectors) may include a promoter operably coupled to the transgene encoding the polypeptide or the polynucleotide encoding the RNA to control expression. The promoter may be a ubiquitous promoter. Alternatively, the promoter may be a tissue specific promoter, such as a myeloid cell-specific or hepatocyte-specific promoter. Suitable promoters that may be used with the compositions described herein include CD11 b promoter, sp146/p47 promoter, CD68 promoter, sp146/gp9 promoter, elongation factor 1 α (EF1α) promoter, EF1α short form (EFS) promoter, phosphoglycerate kinase (PGK) promoter, α-globin promoter, and β-globin promoter. Other promoters that may be used include, e.g., DC172 promoter, human serum albumin promoter, alpha1 antitrypsin promoter, thyroxine binding globulin promoter. The DC172 promoter is described in Jacob, et al. Gene Ther. 15:594-603, 2008, hereby incorporated by reference in its entirety.

The viral vectors (e.g., retroviral vectors, e.g., lentiviral vectors) may include an enhancer operably coupled to the transgene encoding the polypeptide or the polynucleotide encoding the RNA to control expression. The enhancer may include a β-globin locus control region (βLCR).

### Methods of Measuring nORF Gene Expression

Preferably, the compositions and methods of the disclosure are used to facilitate expression of a nORF at physiologically normal levels in a patient (e.g., a human patient), decrease expression of an upregulated nORF, or increase expression of a downregulated nORF. The therapeutic agents ol the disclosure, for example, may reduce nORF expression in a human subject. For example, the therapeutic agents of the disclosure may reduce nORF expression e.g., by about 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 97%, 98%, or 99%.

The expression level ol the nORF expressed in a patient can be ascertained, for example, by evaluating the concentration or relative abundance of mRNA transcripts derived from transcription of the nORF. Additionally, or alternatively, expression can be determined by evaluating the concentration or relative abundance of the nORF following transcription and/or translation of an inhibitor that decreases an amount of the nORF. Protein concentrations can also be assessed using functional assays, such as MDP detection assays. Expression can be evaluated by a number of methodologies known in the art, including, but not limited to, nucleic acid sequencing, microarray analysis, proteomics, in-situ hybridization (e.g., fluorescence in-situ hybridization (FISH)), amplification-based assays, in situ hybridization, fluorescence activated cell sorting (FACS), northern analysis and/or PCR analysis of mRNAs.

### Nucleic Acid Detection

Nucleic acid-based methods for determining expression (e.g., of an RNA inhibitor or an RNA encoding the nORF) detection that may be used in conjunction with the compositions and methods described herein include imaging-based techniques (e.g., Northern blotting or Southern blotting). Such techniques may be performed using cells obtained from a patient following administration of the polynucleotide encoding the agent. Northern blot analysis is a conventional technique well known in the art and is described, for example, in Molecular Cloning, a Laboratory Manual, second edition. 1989, Sambrook, Fritch, Maniatis, Cold Spring Harbor Press, 10 Skyline Drive, Plainview, NY 11803-2500. Typical protocols for evaluating the status of genes and gene products are found, for example in Ausubel et al,, eds., 1995, Current Protocols In Molecular Biology, Units 2 (Northern Blotting), 4 (Southern Blotting), 15 (Immunoblotting) and 18 (PCR Analysis).

Detection techniques that may be used in conjunction with the compositions and methods described herein to evaluate nORF expression further include microarray sequencing experiments (e.g., Sanger sequencing and next-generation sequencing methods, also known as high-throughput sequencing or deep sequencing). Exemplary next generation sequencing technologies include, without limitation, Illumina sequencing, Ion Torrent sequencing, 454 sequencing, SOLiD sequencing, and nanopore sequencing platforms, Additional methods of sequencing known in the art can also be used. For instance, expression at the mRNA level may be determined using RNA-Seq (e.g., as described in Mortazavi et al., Nat. Methods 5:621-628 (2008) the disclosure of which is incorporated herein by reference in their entirety). RNA-Seq is a robust technology for monitoring expression by direct sequencing the RNA molecules in a sample. Briefly, this methodology may involve fragmentation of RNA to an average length of 200 nucleotides, conversion to cDNA by random priming, and synthesis of double-stranded cDNA (e.g., using the Just cDNA DoubleStranded cDNA Synthesis Kit from Agilent Technology). Then, the cDNA is converted into a molecular library for sequencing by addition of sequence adapters for each library (e.g., from Illumina®/Solexa), and the resulting 50-100 nucleotide reads are mapped onto the genome.

Expression levels of the nORF may be determined using microarray-based platforms (e.g., single-nucleotide polymorphism arrays), as microarray technology offers high resolution. Details of various microarray methods can be found in the literature, See, for example, U.S. Pat. No. 6,232,068 and Pollack et al., Nat. Genet. 23:41-46 (1999), the disclosures of each of which are incorporated herein by reference in their entirety. Using nucleic acid microarrays, mRNA samples are reverse transcribed and labeled to generate cDNA. The probes can then hybridize to one or more complementary nucleic acids arrayed and immobilized on a solid support. The array can be configured, for example, such that the sequence and position of each member of the array is known, Hybridization of a labeled probe with a particular array member indicates that the sample from which the probe was derived expresses that gene. Expression level may be quantified according to the amount of signal detected from hybridized probe-sample complexes. A typical microarray experiment involves the following steps: 1) preparation of fluorescently labeled target from RNA isolated from the sample, 2) hybridization of the labeled target to the microarray, 3) washing, staining, and scanning of the array, 4) analysis of the scanned image and 5) generation of gene expression profiles. One example of a microarray processor is the Affymetrix GENECHIP® system, which is commercially available and comprises arrays fabricated by direct synthesis of oligonucleotides on a glass surface. Other systems may be used as known to one skilled in the art.

Amplification-based assays also can be used to measure the expression level of the nORF or RNA in a target cell following delivery to a patient. In such assays, the nucleic acid sequences of the gene act as a template in an amplification reaction (for example, PCR, such as qPCR). In a quantitative amplification, the amount of amplification product is proportional to the amount of template in the original sample. Comparison to appropriate controls provides a measure of the expression level of the gene, corresponding to the specific probe used, according to the principles described herein. Methods of real-time gPCR using TaqMan probes are well known in the art. Detailed protocols for real-time gPCR are provided, for example, in Gibson et al., Genome Res. 6:995-1001 (1996), and in Heid et al., Genome Res. 6:986-994 (1996), the disclosures of each of which are incorporated herein by reference in their entirety. Levels of gene expression as described herein can be determined by RT-PCR technology. Probes used for PCR may be labeled with a detectable marker, such as, for example, a radioisotope, fluorescent compound, bioluminescent compound, a chemiluminescent compound, metal chelator, or enzyme.

### Protein Detection

Expression of the nORF can additionally be determined by measuring the concentration or relative abundance of a corresponding protein product (e.g., the nORF). Protein levels can be assessed using standard detection techniques known in the art. Protein expression assays suitable for use with the compositions and methods described herein include proteomics approaches, immunohistochemical and/or western blot analysis, immunoprecipitation, molecular binding assays, ELISA, enzyme-linked immunofiltration assay (ELIFA), mass spectrometry, mass spectrometric immunoassay, and biochemical enzymatic activity assays. In particular, proteomics methods can be used to generate large-scale protein expression datasets in multiplex, Proteomics methods may utilize mass spectrometry to detect and quantify polypeptides (e.g., proteins) and/or peptide microarrays utilizing capture reagents (e.g., antibodies) specific to a panel of target proteins to identify and measure expression levels of proteins expressed in a sample (e.g., a single cell sample or a multi-cell population).

Exemplary peptide microarrays have a substrate-bound plurality of polypeptides, the binding of an oligonucleotide, a peptide, or a protein to each of the plurality of bound polypeptides being separately detectable. Alternatively, the peptide microarray may include a plurality of binders, including, but not limited to, monoclonal antibodies, polyclonal antibodies, phage display binders, yeast two-hybrid binders, aptamers, which can specifically detect the binding of specific oligonucleotides, peptides, or proteins. Examples of peptide arrays may be found in U.S. Pat. Nos. 6,268,210, 5,766,960, and 5,143,854, the disclosures of each of which are incorporated herein by reference in their entirety.

Mass spectrometry (MS) may be used in conjunction with the methods described herein to identify and characterize expression of the nORF in a cell from a patient (e.g., a human patient) following delivery of the transgene encoding the nORF. Any method of MS known in the art may be used to determine, detect, and/or measure a protein or peptide fragment of interest, e.g., LC-MS, ESI-MS, ESI-MS/MS, MALDI-TOF-MS, MALDI-TOF/TOF-MS, tandem MS, and the like. Mass spectrometers generally contain an ion source and optics, mass analyzer, and data processing electronics. Mass analyzers include scanning and ion-beam mass spectrometers, such as time-of-flight (TOF) and quadruple (Q), and trapping mass spectrometers, such as ion trap (IT), Orbitrap, and Fourier transform ion cyclotron resonance (FT-ICP), may be used in the methods described herein. Details of various MS methods can be found in the literature. See, for example, Yates et al., Annu. Rev, Biomed. Eng. 11:49-79, 2009, the disclosure of which is incorporated herein by reference in its entirety.

Prior to MS analysis, proteins in a sample obtained from the patient can be first digested into smaller peptides by chemical (e.g., via cyanogen bromide cleavage) or enzymatic (e.g., trypsin) digestion. Complex peptide samples also benefit from the use of front-end separation techniques, e.g., 2D-PAGE : HPLC, RPLC, and affinity chromatography. The digested, and optionally separated, sample is then ionized using an ion source to create charged molecules for further analysis. Ionization of the sample may be performed, e.g., by electrospray ionization (ESI), atmospheric pressure chemical ionization (APCI), photoionization, electron ionization, fast atom bombardment (FAB)/liquid secondary ionization (LSIMS), matrix assisted laser desorption/ionization (MALDI), field ionization, field desorption, thermospray/plasmaspray ionization, and particle beam ionization. Additional information relating to the choice of ionization method is known to those of skill in the art.

After ionization, digested peptides may then be fragmented to generate signature MS/MS spectra. Tandem MS, also known as MS/MS, may be particularly useful for analyzing complex mixtures. Tandem MS involves multiple steps of MS selection, with some form of ion fragmentation occurring in between the stages, which may be accomplished with individual mass spectrometer elements separated in space or using a single mass spectrometer with the MS steps separated in time. In spatially separated tandem MS, the elements are physically separated and distinct, with a physical connection between the elements to maintain high vacuum, in temporally separated tandem MS, separation is accomplished with ions trapped in the same place, with multiple separation steps taking place over time. Signature, MS/MS spectra may then be compared against a peptide sequence database (e.g., SEQUEST). Post-translational modifications to peptides may also be determined, for example, by searching spectra against a database while allowing for specific peptide modifications.

### EXAMPLES

The following examples further illustrate the invention but should not be construed as in any way limiting its scope.

### Example 1

Developmental Cycle of P. falciparum and Annotation Challenge

Like many other Apicomplexan parasites, P. falciparum is specialized to infect two separate hosts—the female Anopheles vector mosquitoes that transmit the disease, and the infected human host. The developmental cycle starts with malaria-infected female Anopheles mosquito taking a blood meal, through which the sporozoites of plasmodium parasites are transmitted from the mosquito's salivary gland to the human host (FIG. 7). The sporozoites then travel to the liver and infect liver cells, allowing them to replicate and mature into schizonts. They then rupture and release merozoites, which infect red blood cells and is the stage that causes clinical manifestation of malaria. Some of the blood-stage parasites mature into a schizont, an asexual multiplying form that ruptures and releases more merozoites to infect red blood cells. They can also develop into sexual precursor cells, known as gametocytes, which are ingested by the Anopheles mosquito during a blood meal. The gametocytes are activated by environmental stimuli inside mosquito midgut and differentiate into gametes and fuse to form a zygote. The zygotes further develop into oocysts, within which sporogony takes place and produces sporozoites to invade mosquito's salivary gland, ready for next round of infection via a mosquito bite.

The progression from one developmental stage to another in P. falciparum involves drastic changes in gene expressions, and infectivity of the parasites to human hosts can also vary greatly depending on the stage. For instance, while oocyst sporozoites are highly infectious for the mosquito salivary gland, they are non-infectious to mammalian hosts; on the contrary, salivary gland sporozoites exhibit specific infectivity for mammalian liver, but correspondingly loose infectivity for mosquito's salivary gland.

Proteogenomics and Novel Open Reading Frames (nORFs) Discovery

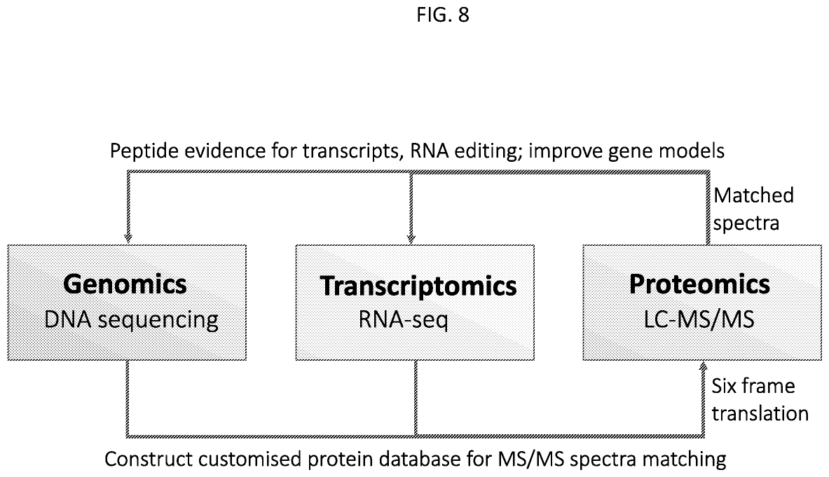

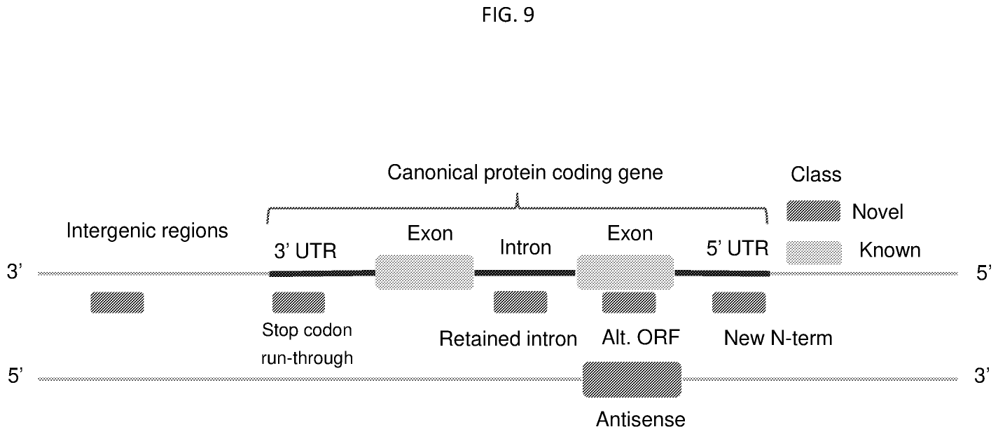

One approach that can address the challenges above is proteogenomics, which combines the power of genomic, proteomic and transcriptomic data to improve genome annotation and our understanding of protein expression (FIG. 8). We performed a peptide search in proteogenomic approach, which allowed the detection of translation of nORFs that may otherwise be neglected, such as those from non-coding regions, including antisense transcripts, IncRNAs, intergenic and intronic sequences, which are defined as shown in FIG. 9. Conversely, the proteomic data can provide peptide evidence for novel transcripts and RNA editing events, further improving the gene models. This approach can be iterative as more multi-omics data are being generated, and continuously improve genome annotation.

Here, we performed proteogenomic analysis on the transcriptomes and proteomes obtained from the oocyst sporozoites and salivary gland sporozoites of P. falciparum to discover novel open reading frames in these two life cycle stages. Bioinformatics analyses suggest that these nORFs are of functional importance.

### Methods

### Dataset

A literature review was conducted to identify the datasets suitable for proteogenomic analysis, which should consist of a pair of proteomes and transcriptomes from the same developmental stage. Thanks to the substantial research efforts devoted to P. falciparum, several transcriptome and proteome datasets have been deposited in the NCBI Sequence Read Archive (NCBI-SRA) and the PRIDE database, respectively. However, it was difficult to find both transcriptomes and proteomes from the same stage, and some are not available in retrievable format. Additionally, omics-data for P. falciparum are usually generated from different, independent studies, making the downstream analysis susceptible to batch effects.

As a result, this work focuses on two pairs of transcriptome/proteome generated by Lindner et al., (Nat. Comm. 10:4694, 2019). The authors produced transcriptomic and proteomic data for oocyst sporozoites from wild-type P. falcparum parasites (NF54 strain) as well as salivary gland sporozoites, with three biological replicates for each sample type. The reasons for narrowing down to these datasets are twofold: firstly, these two stages correspond to the mosquito-infectious and human-infectious stage and therefore novel CRFs discovered by proteogenomic analysis could help explain the development of human-specific infectivity; secondly, since the datasets were produced in the same lab, the conditions should presumably be similar and differential expression is mainly caused by biological differences between the developmental stages. The transcriptomic data was downloaded from the GEO database (Accession #GSM3109291, GSM3109292. GSM3109293 for oocysts sporozoites; # GSM3109294, GSM3109295, GSM3109296 for salivary gland sporozoites), and the proteomic data from PRIDE (Accession #PXD009726 for salivary gland sporozoites, #PXD009728 for oocysts sporozoites).

### Proteogenomic Workflow

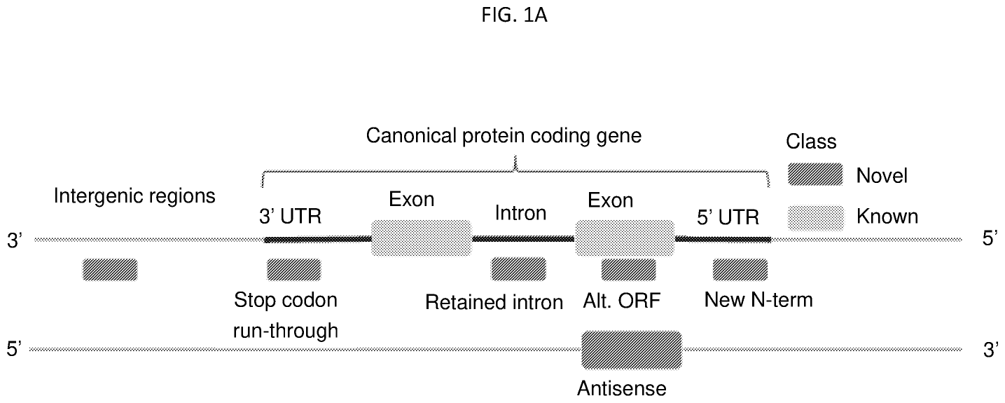

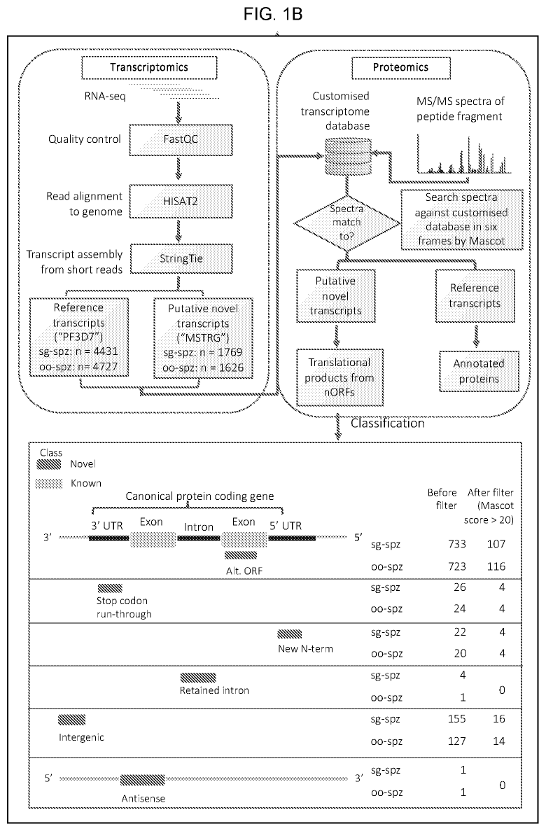

The novelty of proteogenomic analysis comes from matching the MS/MS spectra against a customized database of protein sequences, instead of limiting the searches to known proteins. In this work, a customized database was constructed from the transcripts assembled from the HSAT2-StringTie pipeline (FIGS. 1A and 1B).

Briefly, the quality of RNA-seq data from oocyst sporozoites and salivary gland sporozoites were assessed using FastQC to check for contamination from adapters and the sequences from other species. The adapter sequences were subsequently trimmed using Cutadapt. The processed reads were then aligned to the reference genome for P. falciparum 307 strain retrieved from PlasmoDB ver.46 using HISAT2. The aligned BAM files were used to assemble transcripts using StringTie, which was guided by the reference annotation OFF file (PlasmcDB ver.46) and allowed the assembly of novel transcripts including splice variants. The assembled transcripts therefore include known transcripts from reference annotation with “PF3D7” as transcript ID prefix, and potentially novel transcripts with “MSTRG” prefix, which are constructed from reads that cannot be explained by reference transcripts. Compared to gene-level quantification where all reads are mapped to known genes, transcript-based approach taken by StringTie does not assume that genome annotation is complete and effectively expands the search space for matching MS/MS peptide spectra. The transcript nucleotide sequences were then extracted from the reference genome using BEDTools getfasta. The constructed transcriptome database was then searched in six-frames on the fly for matching to MS/MS spectra by Mascot, a search engine that identifies protein using the MS data. It matches all peptide spectra to the in silico translated proteins derived from the transcriptomic database “on the fly,” hence identifies which transcript is supported by peptide evidence. For each peptide-spectrum match (PSM), Mascot also computes a probability score which is higher for proteins with more peptides matched to it.

### Novel Peptides Classification

Since the known genes have been analyzed thoroughly by Lindner et al. when they reported the transcriptomes and proteomes (Lindner et al., Nat, Comm. 10:4694, 2019), only potentially novel transcripts with “MSTRG” prefix were considered in discovering novel CRFs that have been overlooked previously. Peptides that identify these MSTRG transcripts were subsequently classified (FIG. 1) into different categories as defined above based on the position of matched peptides relative to the reference genes. Each category was classified independently as described below.

### Antisense

The first step to identify antisense peptides was to identify antisense transcripts from the pool of potentially novel MSTRG transcripts. Firstly, all the assembled transcripts were compared with reference transcripts using BEDtools intersect, using parameters that only return transcript that is on the complementary strand of the reference transcript that it overlaps with. Peptides that are matched to these antisense transcripts are then compared with the protein sequences from six frame translation using Transeq to see which frame it originated from. Only peptides that are translated from frame 4 to 6 are considered translation evidence for antisense transcripts, because it could be degenerate peptides from reference genes due to six frame translation.

### Intergenic

The intergenic regions were extracted by using BEDtools complement to subtract the genorne from all annotated regions and return genome intervals with no genes identified. The MSTRG transcripts were mapped to these intervals with BEDtools intersect, returning transcripts that overlap completely are classified as intergenic. Intergenic transcripts with peptides from any frame that identified them are intergenic ORFs.

### Retained Intron

MSTRG transcripts that contain the introns of overlapping reference genes are determined by GffCompare that compares their intron-exon structures. The exons of these transcripts were subsequently intersected with the introns of reference genes to extract the retained intron region, which were six frames translated by Transeq and mapped by the peptides to see if they fall into the introns. We then checked manually if the intronic peptides are in-frame with the neighboring exon on genome visualizer, Artemis; if not, they were classified as AltORFs instead.

### 3′ and 5′ UTR

Unlike other well-characterized genomes, information about the untranslated regions of Plasmodium falciparum is relatively scarce where the majority of research has been focused on coding sequences only. Therefore, the first attempt to extract 3′ and 5′ UTRs by subtracting coding sequence from the genomic coordinates of mRNAs failed, because they are of equal lengths. We combined BEDtools intersect and length filtering to find MSTRG transcripts with complete overlap with a reference gene and are also longer than the overlapped gene. These transcripts therefore contain extended region on the 3′ and/or 5′ end outside coding sequence and regarded as UTRs accordingly. The UTRs were then translated into protein sequence to check if any peptides can confirm their translation. Similarly, peptides from UTRs need to be in-frame with the parent reference gene to be considered, otherwise moved to the AltORFs category.

### AltORF

Along with the out-of-frame peptides from retained intron and UTR classification, all peptides from MSTRG transcripts (excluding antisense transcripts) that overlap with known genes were also compared with the corresponding six frame translated coding sequences. The frame from which the peptides are translated can then be detected, and those from non-canonical frame, were classified as products of AltORFs. However, it is worth noting that almost half of these AltORF peptides map to frames that are antisense (frame 4, 5 and 6) to the canonical genes (341/732 peptides in oocyst sporozoites, and 379/733 peptides in salivary gland sporozoites), these peptides are categorized as AltORF products as they are not associated with an antisense transcript.

### Differential Expression Analysis

To identify novel ORFs that may be involved in the development of infectivity to human hosts, differential expression analysis between the two stages studied was conducted at both transcript and protein levels.

For RNA-seq data, we chose to use the DESeq2 differential expression analysis tool, which is an R package available within the Bioconductor project. Since DESeq2 requires read counts as an input, while StringTie outputs coverage values for transcript abundance, we first converted coverage to counts for each transcript, using the formula reads_per_transcript=coverage*transcript_len/ read_len with a python script (available at ccb.jhu.edu/software/stringtie/dl/prepDE.py). DESeq2 then normalizes the counts internally and compares them between oocyst sporozoites and salivary gland, producing statistics metrics including adjusted p-values and log-transformed fold changes. Transcripts with adjusted p-values<0.1 were called differentially expressed between the two stages.

For protein differential expression analysis, we adopted the spectral count method for protein expression analysis that compares the peptide-spectrum matches (PSM) for each protein between the two stages, which has the highest reproducibility for label-free proteomics data; and we combined it with the G test statistics for computing p-values. However, in this study we matched the MS/MS spectra matched against a customised transcriptome database instead of annotated protein database, we therefore need to adjust the definition of a protein. Because the protein sequence of a translated transcript is different for each frame, we assumed that a given peptide can only identify one frame of the matched transcript being translated, and that a maximum of one protein can be translated from each frame of a transcript. As a result, the sum of PSMs that identity one frame of a transcript are the spectral counts for the protein product from that frame of the transcript.

Based on this assumption, we computed the spectral counts for each frame of each transcript (potential protein) with peptide evidence and increased all of them by 1 to remove zero-values. These counts were subsequently normalized by first calculating the sum of all PSMs of both samples to identify the sample with smaller sum, where its PSMs were multiplied by the ratio of two PSM sums to minimize the background effect between samples. We could then determine the differences in spectral counts of a protein between two stages by applying the C test of significance as follows:

$$G = 2 [ {C}_{oo} ln ( \frac{{C}_{oo}}{( \frac{{C}_{sg +} {C}_{oo}}{2} )} ) + {C}_{sg} ln ( \frac{{C}_{sg}}{( \frac{{C}_{sg +} {C}_{oo}}{2} )} ) ]$$

Where G is the G test static, Cₒₒ and C_{sg} are the normalized spectral counts for a protein in oocyst sporozoite and salivary gland sporozoite respectively. A p-value was calculated as the probability that a X² distribution with 1 degree of freedom was more extreme than the G statistic for that protein. We also used the Benjamini-Hochberg method to correct for false discovery rates from multiple hypothesis testing, where a protein needs to satisfy the criteria of both G-test and FDR<5.0% to be called differentially expressed.

Estimation of Full-Length ORF from Peptide-Spectral Match

While differential expression analysis compares the expression level of one protein between samples, it we also compared the relative abundance of a protein with other proteins within and between samples. Therefore, we computed the normalized spectral abundance factors (NSAF) for each protein, which has been proved to provide reliable quantification. For a given protein k, the spectral abundance factor (SAF) is calculated as the total PSMs (or spectral counts) that identifies the protein normalized by its length, and the NSAF is then the SAF normalized by total SAF values in the sample as shown below:

$${( NSAF )}_{k} = \frac{{( SpC / Length )}_{k}}{{\sum }_{i = 1}^{N} {( SpC / Length )}_{i}}$$

Where (NSAF)ₖ is the NSAF value for protein k, SpC is spectral count and length is the protein length. This method adjusts for the protein length, because larger proteins tend to have higher probabilities of generating more PSMs as well as the total protein abundance in one sample. However, for peptides that do not map to canonical coding sequences, we needed to estimate the open reading frame in the mapped transcript from which they were translated to obtain protein length. The estimated ORFs could also be used for downstream functional analysis.

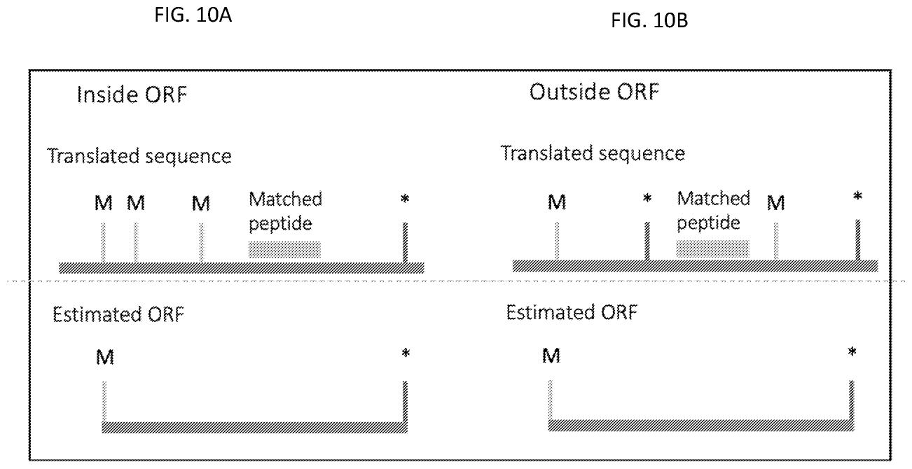

Briefly, for all transcripts with peptide-spectrum matches, we first extracted their spliced nucleotide sequences and performed six frame translation. Then, for each peptide-spectrum match, we identified which frame of the matched transcript it maps to and extract the translated protein sequence. We subsequently determined all possible open reading frames from that sequence defined by the presence of start and stop codon, which is indicated by methionine and respectively in the protein sequence. The matched peptide was then mapped to these possible ORFs and see if it matches to any of them. If not, the ORF will be defined as the sequence flanked by the start and stop codons closet to the matched peptide (FIGS. 10A and 10B). An R script was written to perform this task.

### Gene Ontology Term and Pathway Enrichment Analysis

All categories of novel ORFs analysed in this work except for intergenic ORFs have overlaps with a known gene, We extracted the sets of reference genes overlapped with novel ORFs identified in the oocyst sporozoites and salivary gland sporozoites, respectively, and performed GO term and KEGG pathway enrichment analysis using the Analyze tools on PlasmoDB. InterProScan was used instead to predict GO terms in novel ORFs.

### Structural Prediction

For small proteins with less than 200 amino acids, the structures were predicted using an ab initio structure prediction tool QUARK, which has shown top-ranking performance in the Critical Assessment of Structure Prediction (CASP) experiments consistently. Since ab initio methods work best on small proteins, QUARK has a size limit of 200 amino acids, and larger proteins were predicted using the template-based i-TASSER instead.

### Results

### Identification of Novel Transcripts and Peptides

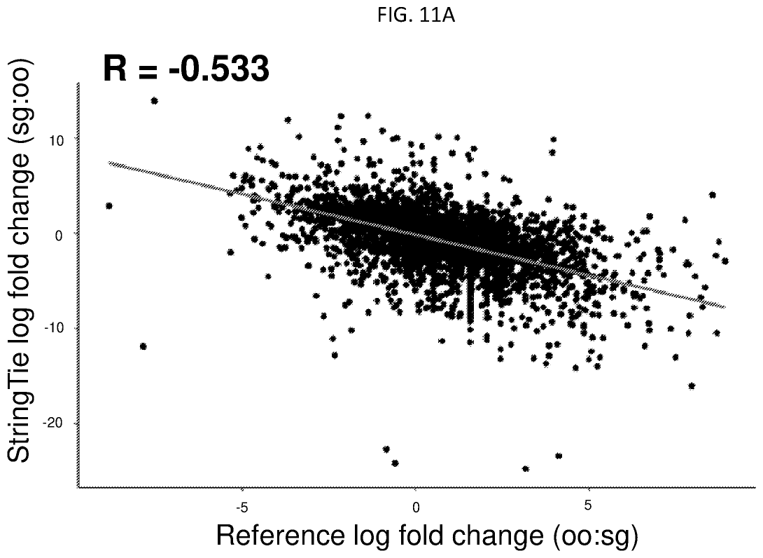

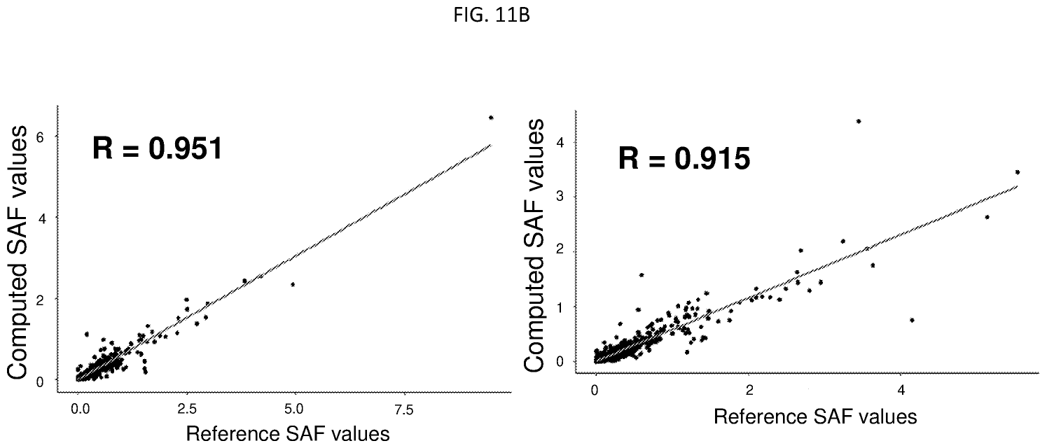

Using the HISAT2-StringTie workflow to align the RNA-seq reads to reference genome and assemble into lull length transcripts, we detected a total of unique 7844 transcripts, with 4727 and 4431 canonical transcripts identified in oocyst sporozoite (oo-spz) and salivary gland sporozoite (sg-spz) stages respectively, which are comparable with the results reported by Lindner et al, (3535 and 3575). The log transformed fold changes of the canonical transcripts also correlate reasonably well with their data (FIG. 11A), with a correlation coefficient of −0.533, where the negative correlation is caused by the stages being compared (oo-spz:sg-spz vs sg-spz:oo-spz). We also identified an additional of 2045 transcripts that are assembled from reads that could not be explained by canonical transcripts (with “MSTRG” transcript prefix). We then classified these potentially novel transcripts into different categories based on their comparison with the overlapped reference genes as described in Methods (FIG. 1). From the MSTRG transcripts, we found 780 and 790 transcripts with 3′ and 5′ UTR regions respectively that are unseen in canonical transcripts, 326 transcripts with retained introns, 41 antisense transcripts, and 427 intergenic transcripts. Identification of AltORFs requires translational evidence and are therefore not applicable. The high abundance of transcripts from the UTR and intergenic regions agrees with a recent study that reported nearly 90% of the Plasmodium falciparum is being actively transcribed. Our results together suggest that these untranslated regions that could not have been identified using traditional methods are likely to have some biological functions. Therefore, the gene boundaries need to be re-defined to take the regions outside coding sequence into consideration.

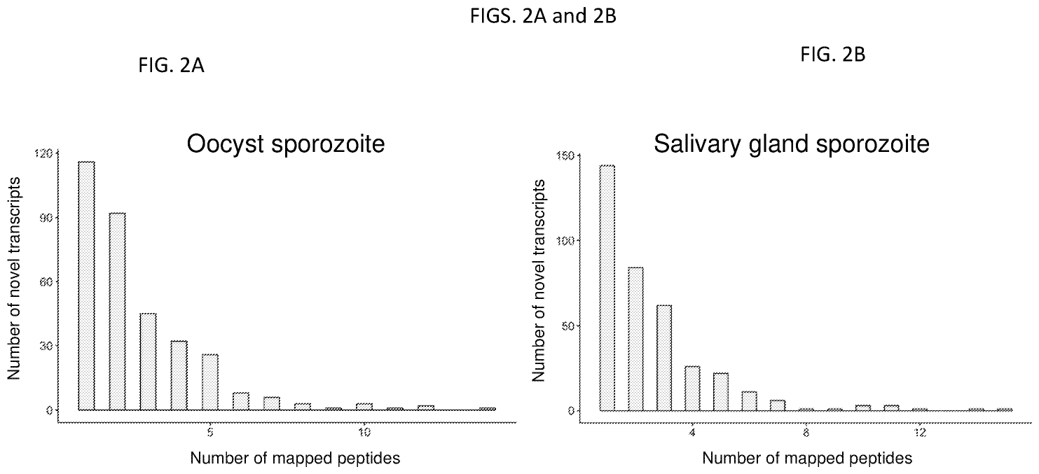

However, transcriptional evidence alone tends to be very noisy, especially for non-canonical transcripts that are constructed from de novo assembly. With the availability of proteomic data, we were able to use the PSMs from novel transcripts to identify their existence with much higher confidence. As shown in FIG. 2A and 2B, most of the novel transcripts have one or two unique peptides mapped to them, with very rare cases of over ten peptides. This suggests that for these novel transcripts, only one frame is translated even with six-frame translation and the translated region probably covers a small segment of the transcript.

Furthermore, a relatively small fraction of novel transcripts has translation evidence. Less than ⅓ of the intergenic transcripts from the two stages have peptide spectral matches that can identify them (n−130), although many of them are specific to either salivary gland or oocyst sporozoites (Table 1). Notably, despite many transcripts showed retained introns of canonical genes, only five of them have peptide evidence that supports the translation of these introns, indicating that the current intron-exon structures of coding sequences are likely to be accurate. Similarly, antisense peptides were detected at a very low level, with one PSM in each stage. Unlike other untranslated regions, we have detected slightly more novel peptides translated from the 3′ and 5′ UTRs. One explanation is that they reflect the incomplete annotation of reference genes, or they could arise from stop-codon run-through and translation of upstream ORFs respectively, where the latter might regulate the expression of downstream canonical ORFs. However, despite the low translation level of these novel transcripts, their regulatory roles on canonical proteins may be at RNA level only, especially antisense transcripts, whose RNA molecules were shown to interact with chromatin and regulate gene expression in P. falciparum.

The majority of the novel peptides belong to the alternative ORF (AltORF) category, which was defined as the translational products from a non-canonical reading frame of the overlapped coding sequence, including those that fail into introns and the 3′ and 5′ UTR regions of the transcript. AltORFs have previously been found in viruses, bacteriophages as well as humans, but to our knowledge their existence has not been reported in P. falciparum yet. This suggests that the proteome of P. falciparum may be much more complicated than previously thought, and the abundance of AltORFs suggests that the parasite may use it to expand the coding potential of existing genes in the compact genome. Although the functions of AltORFs remain largely unknown, it was thought that the translation of these non-canonical ORFs alone could provide a mechanism for expression control. It is therefore interesting to investigate if these novel peptides could explain the development of infectivity for human host in the parasite.

TABLE 1
Number of novel transcripts identified from RNA-seq data and
number of novel transcripts with translational evidence

| | | | Retained | | | | |
|---|---|---|---|---|---|---|---|
| | Antisense | Intergenic | intron | 3′ UTR | 5′ UTR | AltORF | |
| | 41 | 427 | 326 | 780 | 790 | / | Novel |
| | | | | | | | transcript |
| Salivary gland | 1 | 87 | 4 | 24 | 21 | 261 | Novel |
| sporozoite | | | | | | | transcript |
| Oocyst | 1 | 64 | 1 | 22 | 18 | 261 | with |
| sporozoite | | | | | | | peptides |
| Transcript | 0 | 21 | 0 | 7 | 8 | 132 | |
| common in | | | | | | | |
| both stages | | | | | | | |
| Total unique | 2 | 130 | 5 | 39 | 31 | 390 | |
| transcript | | | | | | | |

### ORF Prediction and Differential Expression Analysis

To study the regulatory roles of the novel peptides, we first determined if their parent proteins were differentially expressed between oocyst sporozoites and salivary gland sporozoites. However, unlike most canonical proteins whose amino acid sequences have been identified experimentally, novel peptides themselves do not provide information of the full-length novel proteins from which they were generated, they are only evidence that there are PSMs that identify a segment of the transcript. We therefore attempted to predict the open reading frame based on where the peptides map to translated sequence of transcripts (see Methods).

We applied this approach to both putative novel proteins and canonical proteins, where the latter have annotated protein lengths that allow us to verify the feasibility of estimating full-length ORFs from PSMs of six-frame translated transcripts. By searching the MS/MS spectra against the customized transcriptome database, we identified 2901 and 2933 canonical proteins in oocyst sporozoite and salivary gland sporozoite respectively, as compared to 1432 and 2040 proteins identified by Lindner et al. We then compared our results for the protein-length normalized spectral abundance factors (SAF) for canonical proteins with those reported by Lindner et al. The protein abundances (in SAF) that we computed for canonical proteins in the samples from two stages correlated strongly with the published data (FIG. 11B), with correlation coefficients of 0.95 and 0.91 for oocyst sporozoite and salivary gland sporozoite respectively, indicating that we could deduce the ORFs for novel peptides using this method (FIGS. 10A and 10B) as well.

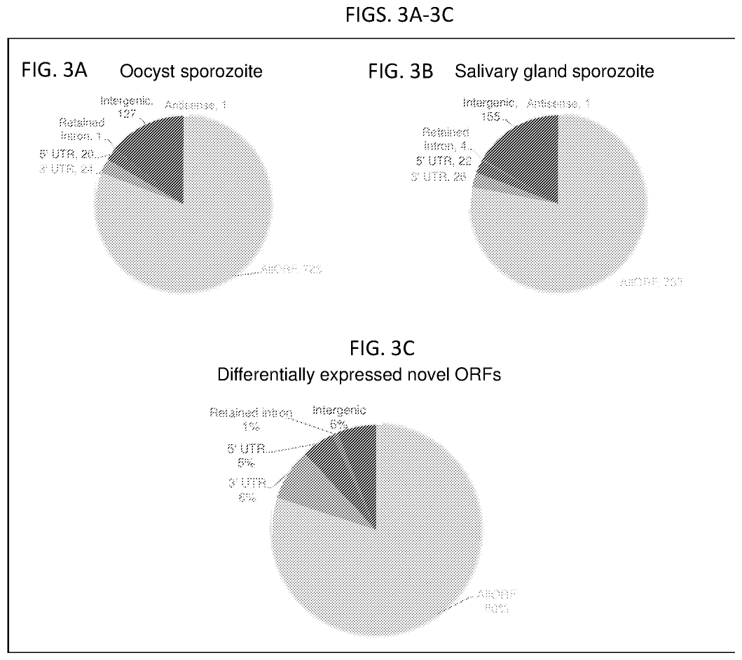

After computing the PSMs for both novel and canonical proteins in two different stages, we could then proceed to identify the ones that were differentially expressed using G-test statistics. In total, we observed 86 differentially expressed novel ORFs, which share a similar distribution of nORF categories with those present in the total proteomics data of salivary gland sporozoites and oocyst sporozoites (FIGS. 3A-3C). One exception is the intergenic ORFs—while the intergenic peptides were the second most abundant category in both stages, only 6% of the differentially expressed nORFs are from the intergenic region.

### Antisense Transcripts and Peptides

Although only two antisense peptides were identified in this study, antisense transcripts have previously been shown to be incorporated into chromatin and consequently activate the virulence factor in P. falciparum, the vargene family. It is therefore worth investigating further the regulatory roles of the antisense transcripts on their parent transcripts. We first extracted the corresponding reference genes of the 41 antisense transcripts, then calculated the transcript abundances in TPM (Transcripts Per Million) of both sense and antisense transcripts, and finally compared the TPM values using Pearson correlation. As a result, five antisense transcripts were shown to have significant correlation (p<0.05) with the sense reference transcripts (Table 2), where three of them have positive correlation that suggests an activation mechanism. interestingly, regardless of the transcript correlation, all reference genes with associated antisense transcripts are significantly downregulated at transcript level in salivary gland sporozoites. Especially for the two antisense transcripts that showed anticorrelated expression, one of the parent gene has both downregulated mRNA and protein expression, while the other is downregulated at mRNA level but upregulated at protein level. Notably, the MSTRG.401.1 transcript also has translational evidence, suggesting that the regulation by antisense transcripts could be mediated by their translational product. Overall, we observed that the actions of antisense transcripts are not uniform where they could potentially regulate the expression of their target genes not just by activation but also by repression. However, since the transcript level of the parent reference genes do not correlate well with protein expression, there might be other mechanisms involved.

TABLE 2
Correlation of TPM values between antisense transcripts
and their associated reference transcripts.

| | | | | Reference | Reference |
|---|---|---|---|---|---|
| Associated | Antisense | Correlation | | transcript | protein |
| reference transcript | transcript | coefficient | p-value | fold-change | fold-change |
| PF3D7_0209600.1 | MSTRG.182.1 | 0.984 | 0.000360 | ↓ | ns |
| PF3D7_0103800.1 | MSTRG.17.1 | −0.964 | 0.00187 | ↓ | ↓ |
| PF3D7_0414500.1 | MSTRG.618.1 | 0.947 | 0.00415 | ↓ | ns |
| PF3D7_0314000.1 | MSTRG.401.1 | −0.928 | 0.00758 | ↓ | ↑ |
| PF3D7_1116000.1 | MSTRG.2636.1 | 0.879 | 0.0211 | ↓ | ↓ |

↓ indicates that the fold change is negative in differential expression analysis and ↑ indicates positive fold-change.
ns indicates that the reference transcript was not significantly differentially expressed

### GO Term and Pathway Enrichment

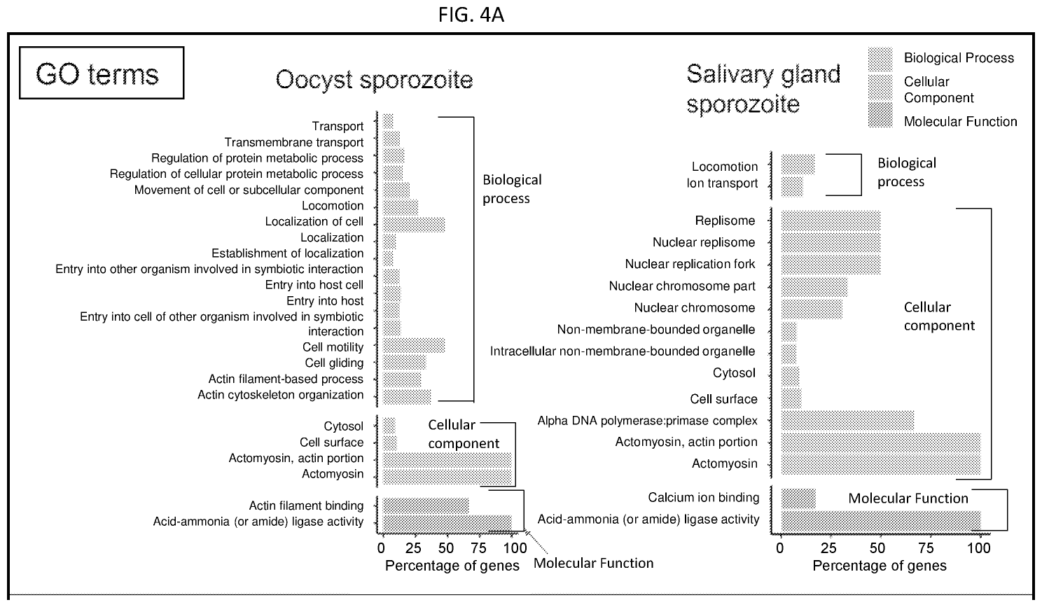

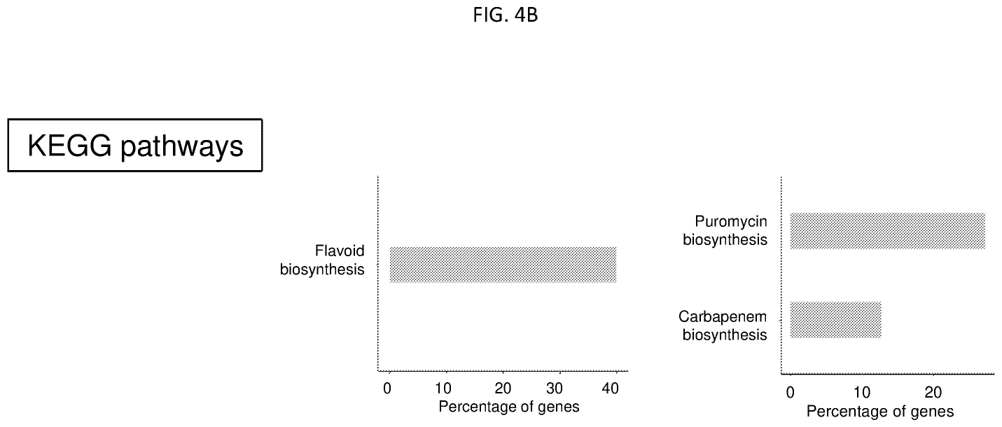

To investigate the functional roles of the loci from which the novel peptides arise, we performed enrichment analysis on the GO terms and KEGO pathways on their parent genes. As shown in FIGS. 4A and 4B, the novel peptides identified from oocyst sporozoites and salivary gland sporozoites are associated with very different sets of known genes. With a stringent p-value cut-off of 0.01, the parent genes that overlap with the novel peptides in oocyst sporozoites have a clear enrichment in processes related to cell localization and movement, which is further supported by the 100% enrichment of the background genes in actomyosin. On the other hand, the gene set for salivary gland sporozoite is also enriched in the actomyosin structure, and distinctively enriched in replisome complex as well.

Since oocyst sporozoites need to migrate from mosquito midgut to salivary gland and await injection into human host via a blood meal, motility is crucial for host invasion and achieved by the invasion machinery called glideosome, which is powered by the actomyosin system. T

[symbol]

For KEGG pathway enrichment, we chose to use a less stringent cut-off because otherwise no pathways could pass the filter, which still returned very few enriched pathways (FIG. 4B), Interestingly, the gene set in salivary viand sporozoite is enriched in the pathway for the biosynthesis of an antibiotic, puromycin, where the genes involved in this pathway are all alpha/beta hydrolases, Of particular interest is the BEM 46-like protein, which was shown to modulate the development of sporozoites, and therefore the associated novel peptides may play a role in the modulation as well,

### High Quality Peptide Filtering and Re-Analysed Differential Expression

To obtain high quality novel peptides, we applied a quality filter of Mascot score>20 on the proteomic datasets and subsequently performed differential expression analysis on the proteins of filtered peptides. Approximately half of the total peptides remained after applying the filter (Table 3), whereas for novel peptides the proportions are much lower, ranging from 14% to 20%. This suggests that compared with peptides from canonical proteins, novel peptides tend to have lower probabilities of being a real positive match. A total of 138 and 131 unique novel peptides remained in the occyst and salivary gland sporozoite dataset respectively (Table 4) after the filtering, with AItORF being the most common category followed by intergenic, which follows a similar trend before filtering. Unfortunately, no peptides from antisense strand or retained intron passed the filter, which were very rare before filtering too.

We then estimated the ORFs of the filtered novel peptides and observed which of them was differentially expressed. With a much smaller pool of high-quality peptides, only five unique novel ORFs showed differential expression between the two stages (Table 5), where AItORF is still the most common category, and one ORF from 3′ UTR and intergenic regions. interestingly, four out of five DE nORFs showed opposite trend in transcript and protein expression, meaning that those that have positive fold-change at mRNA-level showed negative fold-change at protein level and vice versa. As a result, a downregulated transcript can have upregulated protein expression due to the removal of repression.

TABLE 3
Percentage of novel peptides and total peptides
passing the filter of Mascot score >20.

| | Percentage of | Percentage of |
|---|---|---|
| | novel peptides | total peptides |
| Sample | passing the filter | passing the filter |
| oo1 | 17.5 | 50.5 |
| oo2 | 19.8 | 49.7 |
| oo3 | 15.2 | 48.2 |
| sg1 | 14.5 | 53.0 |
| sg2 | 19.9 | 53.4 |
| sg3 | 17.7 | 52.8 |

oo1, oo2, oo3 represent the three biological replicates from oocyst sporozoite and sg1, sg2, sg3 are the replicates from salivary gland sporozoite

TABLE 4
Number of novel peptides from different categories
that passed the quality filter of Mascot score >20.

| | Number of unique novel peptides | | | |
|---|---|---|---|---|
| | 5′ UTR | 3′ UTR | AltORF | Intergenic |
| Oocyst sporozoite | 4 | 4 | 116 | 14 |
| Salivary gland sporozoite | 4 | 4 | 107 | 16 |

TABLE 5
High quality (Mascot score >20) novel ORFs identified by using filtered peptides only.

| | | Category | PSM in | PSM in | Log ratio | Log fold-change | |
|---|---|---|---|---|---|---|---|
| Transcript ID | Frame | of nORF | oo-spz | sg-spz | of PSMs | of transcripts | p-value |
| MSTRG.2270.1 | 1 | 3p UTR | 3.32 | 16.00 | 2.27 | −1.74 | 0.0026 |
| MSTRG.231.2 | 1 | AltORF | 28.75 | 1.00 | −4.85 | 11.57 | 0.0000 |
| MSTRG.633.1 | 6 | AltORF | 22.12 | 2.00 | −3.47 | ns | 0.0000 |
| MSTRG.4174.1 | 2 | Intergenic | 1.11 | 11.00 | 3.31 | −3.49 | 0.0022 |
| MSTRG.4394.1 | 1 | AltORF | 1.11 | 15.00 | 3.76 | −3.34 | 0.0002 |

PSM values were normalized as described in Methods. MSTRG.4174.1 (SEQ ID NO: 27)

### Differentially Expressed 3′ UTR

It was worth noting that the differentially expressed 3′ UTR (MSTRG.2270.1 ; SEQ ID NO: 28), despite showing higher expression in salivary gland sporozoites, the peptide spectrum-match that maps to the 3″-end of the canonical gene (gene identifier: PF3D7_1013400) was only present in oocyst sporozoites. This is in line with the RNA-seq data, where the 3′-end is clearly expressed in oocyst sporozoites but almost undetectable in salivary gland sporozoite. Even though there were only two PSMs from this nORF before normalization in oocyst sporozoites, the 3′ UTR was still captured at such low expression, it is unlikely that the absence of peptide from 3′ UTR in salivary gland sporozoites is due to chance or physical chemistry of the peptide ion. It is therefore possible that the protein with 3′UTR is an isoform specific to the oocyst sporozoite stage.

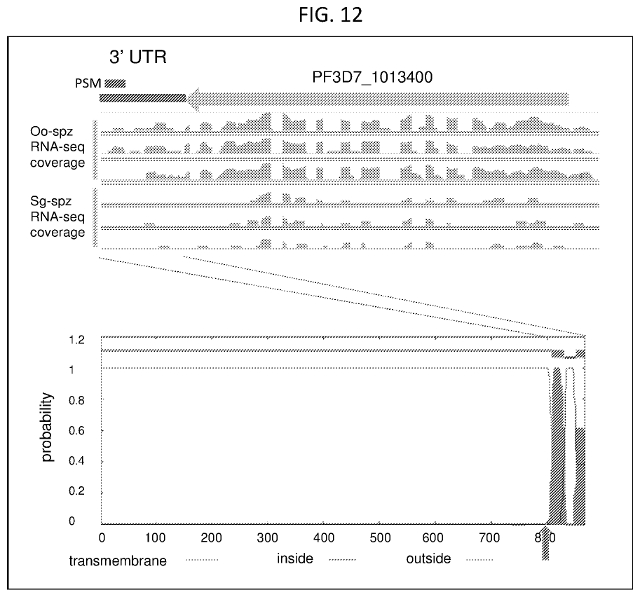

To understand the function of the extended 3′UTR region, we used InterProScan to analyze the sequence of this nORF. The results revealed that while the canonical gene PF3D7_1013400 has only a long stretch of “Non-cytoplasmic domain” identified, the extended 3′-end of 71 amino acids was predicted with multiple transmembrane helices. This is further confirmed by the prediction of transmembrane helices from TMHMM, which predicted two helices in the region after the canonical stop codon (FIG. 12). it also suggested that canonical protein is likely to be outside of the membrane, which coincides with the non-cytoplasmic domain detected by InterProScan. Therefore, the extended region in the 3′ end is likely to act as an anchor that tether the canonical protein to the membrane in oocyst sporozoites. When the parasite transitions into salivary gland sporozoite, the canonical stop codon is used and without expressing the 3′ end of transmembrane helices.

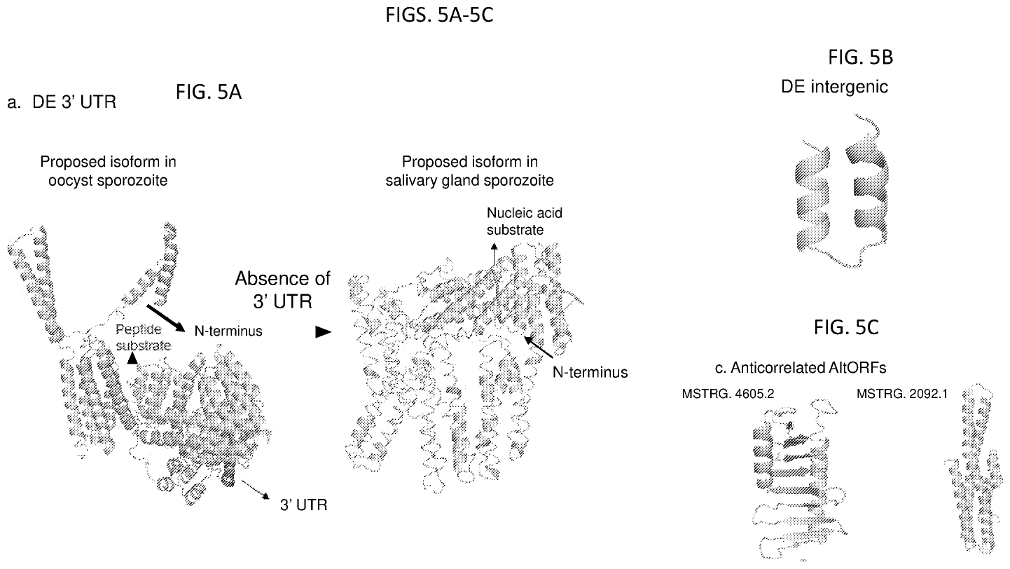

Since there is no function annotated for the parent gene of this 3′UTR other than it encodes a conserved protein, we predicted its structures with and without the 3′ extension using the template-based I-TASSER. It appears that the isoforrn with extra helices at the 3′-end (FIG. 5A) has a very different structure than the one without, where the former is predicted to have a more compact, ordered structure and likely to bind to a peptide substrate, while the latter contains a lot of disordered loops with nucleic acid substrate. Therefore, it is possible that the presence of extra helices allows the parent protein to tether to the membrane and adopt a more stable structure to bind to different substrates. When the parasite matures into salivary gland sporozoite, it expresses the canonical protein with no transmembrane helices, which may be released into the extracellular environment given its non-cytoplasmic domain and potentially involved in the host-parasite interaction upon infection.

### Intergenic ORF and Transmembrane Domains

Surprisingly, an intergenic nORF was also differentially expressed after the quality filtering, It is a short ORF with only 30 amino acids and unique to salivary gland sporozoites, identified by 10 raw PSMs (Table 5). InterProScan could not detect any functional domain possibly due to its short length, and therefore we modelled its structure using an ab initio structure prediction tool QUARK, which can yield high-resolution structures for small proteins. The predicted structure (FIG. 5B) is highly ordered with two short helices connected by a loop, suggesting that the intergenic OAF could form protein-like products too.

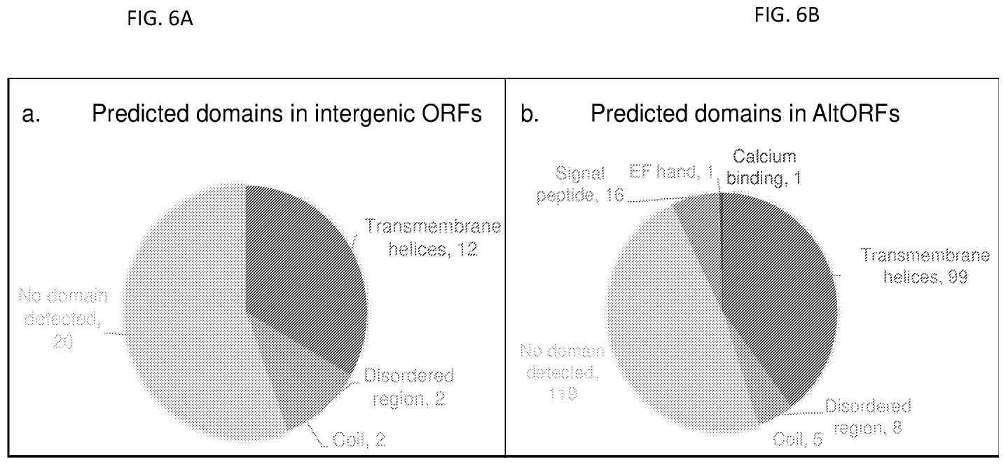

We then tried to analyze other intergenic ORFs with high-quality PSMs and see if they have functional roles. Firstly, we performed BlastP search on all the intergenic ORFs against the non-redundant protein sequence database, and no significant hit with expected value (E-value) smaller than 0.05 was returned, indicating that these ORFs share little or no sequence homology with known proteins. Interestingly, InterProScan detected many transmembrane domains in the intergenic ORFs from prediction by TMHMM (FIG. 6A), where 12 out of 34 were predicted with one or more transmembrane helices, and one of them was predicted with four helices. A similar scenario was observed in AItORFs (FIG. 6B) as wet, where 99 out of 248 ORFs were predicted to contain transmembrane domain. Such abundance suggests that these ORFs may have biological functions in the membrane, which have been previously overlooked by the conventional annotation methods. We speculate that this may be a common strategy adopted by organisms with small genomes to expand their proteome, although their exact functional roles still require experimental testing.

Correlation of nORF Expression with Canonical Gene

Finally, we tested if the protein expression of high-quality nORFs correlate with that of their associated canonical gene. By computing the normalized spectral abundance factor (NSAF) values, we were able to compare the expression across proteins and samples, which were used to perform Pearson Correlation analysis. in total, we identified nORFs (Table 6) that showed significant expression correlation (p-values<0.05) with the parent gene, which are all AltORFs except for one 3′ UTR. It is worth noting that majority of them showed positive correlation, suggesting that either a positive regulation on the parent gene is a wide-spread scenario in nORFs, or that their expression is a by-product of the canonical translation event via mechanisms such as ribosome shifting,

Interestingly, two AltORFs (MSTRG 4605.2 and MSTRG 2092.1) have anti-correlated expression with the parent gene, which are less likely to be a biproduct and more likely have regulatory role on the expression of canonical proteins. Moreover, one of the parent genes, PF3D7_1467600 was significantly downregulated in salivary gland sporozoite, where the associated AltORF is uniquely present, suggesting that it may be involved in the downregulation mechanism. InterProScan did not find any functional domains other than transmembrane helices in the two AltORFs, which was common for novel ORFs as previously discussed. Therefore, we attempted to infer function from their three-dimensional structures by first predicting the structures using QUARK (FIG. 5C), and then submitting them to the structure-based function predictor COFACTOR (Roy et al., 2012). Although there were no significant hits (C-score>0.4) of the predicted Molecular Function GO terms for MSTRG 4605.2 (Table 7), MSTRG 2092.1 was predicted with nucleic acid binding function. Therefore, binding to the mRNA of canonical gene or to the chromosome could be a mechanism through which this AltORF regulates the expression of canonical proteins.

TABLE 6
High quality novel ORFs that have significantly (p-value < 0.05) correlated
protein expression (calculated in NSAF) with their associated reference genes

| | | Category | | | Correlation | Nucleotide | Peptide |
|---|---|---|---|---|---|---|---|
| Transcript ID | Frame | of nORF | Reference gene | p-value | coefficient | SEQ ID NO. | SEQ ID NO. |
| MSTRG.4605.2 | 2 | AltORF | PF3D7_1467600 | 0.0251 | −0.868 | 1 | 14 |
| MSTRG.2092.1 | 6 | AltORF | PF3D7_0927900 | 0.0425 | −0.827 | 2 | 15 |
| MSTRG.1076.2 | 2 | AltORF | PF3D7_0606600 | 0.0480 | 0.815 | 3 | 16 |
| MSTRG.1556.1 | 2 | AltORF | PF3D7_0728700 | 0.0457 | 0.820 | 4 | 17 |
| MSTRG.3171.1 | 1 | AltORF | PF3D7_1225600 | 0.0367 | 0.839 | 5 | 18 |
| MSTRG.2151.1 | 6 | AltORF | PF3D7_0934500 | 0.0149 | 0.898 | 6 | 19 |
| MSTRG.2472.1 | 2 | 3p UTR | PF3D7_1033500 | 0.0134 | 0.904 | 7 | 20 |
| MSTRG.2472.1 | 6 | AltORF | PF3D7_1033500 | 0.0134 | 0.904 | 8 | 21 |
| MSTRG.4173.1 | 2 | AltORF | PF3D7_1417600 | 0.00838 | 0.924 | 9 | 22 |
| MSTRG.370.1 | 1 | AltORF | PF3D7_0311100 | 0.00560 | 0.938 | 10 | 23 |
| MSTRG.3602.1 | 6 | AltORF | PF3D7_1322400 | 0.00098 | 0.974 | 11 | 24 |
| MSTRG.4140.1 | 1 | AltORF | PF3D7_1414500 | 0.000685 | 0.979 | 12 | 25 |
| MSTRG.2185.1 | 2 | AltORF | PF3D7_1004300 | 0.000073 | 0.993 | 13 | 26 |

TABLE 7
Predicted molecular functions of the structures
modelled by QUARK for MSTRG.4605.2 and MSTRG.2092.1,
which showed negative correlation of protein expression
with associated reference gene.

| MSTRG.4605.2 | | MSTRG.2092.1 | |
|---|---|---|---|
| Molecular function | C-score | Molecular function | C-score |
| Phosphatidic acid binding | 0.13 | Nucleic acid binding | 0.46 |
| Phosphatidylinositol-4- | 0.13 | Phosphatase binding | 0.40 |
| phosphate binding | | | |
| Sterol transporter activity | 0.13 | Nucleic acid binding | 0.40 |
| | | transcription factor | |
| | | activity | |
| Oxysterol binding | 0.13 | Structural constituent | 0.28 |
| | | of cytoskeleton | |
| Phosphatidylinositol-4,5- | 0.13 | Catalytic activity | 0.26 |
| bisphosphate binding | | | |

C-scores are confidence scores that range from 0 to 1, which higher score indicating a more confident prediction

### Discussion

We performed proteogenomic analysis to discover novel open reading frames that would not have been identified using conventional approaches and could enhance our understanding in the parasite biology. The datasets analyzed are the total transcriptomes and proteomes of oocyst sporozoites and salivary gland sporozoites, which correspond to the life-cycle stages that are mosquito-infectious and human-infectious respectively, and therefore the identified nORFs could contribute to the understanding of the development of infectivity.

In this work, we classified putative novel transcripts based on how they map to the canonical genes and subsequently determined where their peptide-spectrum matches align to the translated sequence to identify novel peptides. In totally, we identified a total of 1734 novel peptides, where 269 of them passed the high-quality filter of Mascot score>20. We also developed a method to predict the full-length open reading frame from peptides by finding the most suitable start and stop codons in the translated transcript, so that differential analysis could be performed for novel ORFs. Our results for the length-normalized spectral abundance factors of canonical genes correlated well with those reported by Lindner et al. (FIG. 11B), which were computed using annotated protein lengths, suggesting that our approach is feasible.

By performing GO terms enrichment analysis on the canonical genes associated with the novel ORFs, it appears that they arise from functionally important loci that are critical to the parasite invasion and survival. While the gene set associated with novel ORFS identified in oocyst sporozoites was enriched in GO terms of motility and localization, the gene set for salivary gland sporozoites was more enriched in replisome-related GO terms (FIG. 4A). More importantly, both gene sets showed significant enrichment in the genes that form the actomyosin complex, which is part of the parasite invasion machinery, also known as the glideosome. It is therefore possible that these novel ORFs play a role in host invasion and could serve as a drug target as well.

The functional roles of antisense transcripts align with the observations where the levels of the 41 identified antisense transcripts do not have a consistent correlation with protein expression. Interestingly, we observed some translation of the antisense transcripts, suggesting that their regulatory roles could be performed by the translation product as well.

We found that while a lot of assembled transcripts have extended 3′/5′ ends and retained introns, very few peptides are mapped to these untranslated regions. Nevertheless, these UTRs tend to be regulatory elements that function at mRNA level and very little is known about their roles in transcriptional and translational regulation in the Plasmodium parasite, our results could provide a starting point for further investigation. Furthermore, we identified one high quality novel ORF from the 3′ UTR category that was differentially expressed between the two stages studied, and the peptide that maps to 3′ end was only observed in oocyst sporozoite despite low protein expression. The extra sequences provided by the 3′ extension are predicted to form a transmembrane helix and could affect the protein structure significantly, which may therefore allow the protein to attach to the membrane and bind to different substrates. We speculate that the parasite may choose to use different protein isoforms depending on the life cycle stage by skipping a stop codon and express the 3′ end, which could be an efficient mechanism to change infectivity for different hosts.

Finally, AltORFs and intergenic ORFs are surprisingly abundant, with AItORFs being the most common category in both stages as well as in differentially expressed novel ORFs. By correlating the nORF expression with their associated canonical genes, we found that most nORFs that have significantly correlated expression are AltORFs with positive correlation, except for two AltORFs that showed negative correlation. Structural analysis reveals that one of them is likely to form a protein structure that binds to nucleic acid, which provides a possible mechanism for this AltORF to regulate the expression of the associated gene by repressing its transcription or the translation of mRNA. On the other hand, we found that intergenic ORFs were not only translated, some of them were even differentially expressed between cocyst and salivary gland sporozoites, suggesting that they might be involved in the stage-specific functions. An intriguing finding is that 12 out of 34 high-quality intergenic ORFs, and 99 out of 248 AlTORFs were predicted with transmembrane domain, which may indicate that they have an important functional role in the membrane. Given that a similar scenario has been observed in Escherichia coli, non-canonical transmembrane ORFs might be common in organisms with small genomes to expand the functional proteome.

In summary, we have identified the novel ORFs in oocyst sporozoites and salivary gland sporozoites through proteogenomic analysis, which allowed us to explore transcription and translation events outside the coding sequences that are annotated using conventional criteria. Combining analyses of differential expression, GO term enrichment and predicted structures, we have shown that they are likely to have important functional roles in the parasite invasion.

### OTHER EMBODIMENTS

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

1. A method of treating malaria in a subject comprising:
(a) identifying a sequence of a novel open reading frame (nORF) in Plasmodium falciparum, wherein the sequence of the nORF is distinct from a canonical open reading frame (cORF) of a gene, wherein the nORF is present in (i) an overlapping region of the cORF in an alternate reading frame, (ii) a 5′ untranslated region (UTR) of the cORF. (iii) a 3′ UTR of the cORF. (iv) an intronic region of the cORF, (v) an intergenic region of the cORF, or (vi) a region not associated with the cORF or the gene, or in an antisense transcript thereof, wherein the nORF is expressed in Plasmodium falciparum; and
(b) administering to the subject an inhibitor that reduces expression of the nORF to treat malaria.

2. A method of treating malaria in a subject comprising administering to the subject an inhibitor that reduces expression of a nORF in Plasmodium falciparum; wherein the subject has previously been identified with a sequence variant of the nORF, wherein the sequence of the nORF is distinct from a cORF of a gene, wherein the nORF is present in (i) an overlapping region of the cORF in an alternate reading frame, (ii) a 5′ UTR of the cORF, (iii) a 3′ UTR of the cORF, (iv) an intronic region of the cORF, (v) an intergenic region of the cORF, or (vi) a region not associated with the cORF or the gene, or in an antisense transcript thereof, and wherein the nORF is expressed in Plasmodium falciparum,

3. The method of claim 1 or 2, wherein the nORF is expressed in a mosquito and in the subject.

4. The method of claim 3, wherein the nORF is expressed in an oocyst sporozoite and a salivary gland sporozoite.

5. The method of any one of claims 1 to 4, wherein the inhibitor comprises a small molecule, a polynucleotide, or a polypeptide.

6. The method of claim 5, wherein the polynucleotide comprises a miRNA, an antisense RNA, an shRNA, or an siRNA,

7. The method of claim 5, wherein the polypeptide comprises an antibody or antigen-binding fragment thereof.

8. The method of claim 7, wherein the antigen-binding fragment thereof is an scFv.

9. The method of any one of claims 5 to 8, wherein the inhibitor is encoded by a vector.

10. The method of claim 9, wherein the vector is a viral vector.

11. The method of claim 10, wherein viral vector is selected from the group consisting of a Retroviridae family virus, an adenovirus, a parvovirus, a coronavirus, a rhabdovirus, a paramyxovirus, a picornavirus, an alphavirus, a herpes virus, and a poxvirus.

12. The method of claim 11, wherein the parvovirus viral vector is an adeno-associated virus (AAV) vector.

13. The method of claim 12, wherein the viral vector is a Retroviridae family viral vector.

14. The method of claim 13, wherein the Retroviridae family viral vector is a lentiviral vector.

15. The method of claim 13, wherein the Retroviridae viral vector is an alpharetroviral vector or a gammaretroviral vector.

16. The method of any one of claims 12 to 15, wherein the Retroviridae family viral vector comprises a central polypurine tract, a woodchuck hepatitis virus post-transcriptional regulatory element, a 5′-LTR, HIV signal sequence, HIV Psi signal 5′-splice site, delta-GAG element, 3′-splice site, and a 3′-self inactivating LTR.

17. The method of any one of claims 12 to 16, wherein the viral vector is a pseudotyped viral vector.

18. The method of claim 17, wherein the pseudotyped viral vector is selected from the group consisting of a pseudotyped adenovirus, a pseudotyped parvovirus, a pseudotyped coronavirus, a pseudotyped rhabdovirus, a pseudotyped paramyxovirus, a pseudotyped picornavirus, a pseudotyped alphavirus, a pseudotyped herpes virus, a pseudotyped poxvirus, and a pseudotyped Retroviridae family virus.

19. The method of claim 18, wherein the pseudotyped viral vector is a lentiviral vector,

20. The method of any one of claims 17 to 19, wherein the pseudotyped viral vector comprises one or more envelope proteins from a virus selected from vesicular stomatitis virus (VSV). RD114 virus, murine leukemia virus (MLV), feline leukemia virus (FeLV), Venezuelan equine encephalitis virus (VEE), human foamy virus (HFV), walleye dermal sarcoma virus (WDSV), Semliki Forest virus (SFV), Rabies virus, avian leukosis virus (ALV), bovine immunodeficiency virus (BIV), bovine leukemia virus (BLV), Epstein-Barr virus (EBV), Caprine arthritis encephalitis virus (CAEV), Sin Nombre virus (SNV), Cherry Twisted Leaf virus (ChTLV), Simian T-cell leukemia virus (STLV), Mason-Pfizer monkey virus (MPMV), squirrel monkey retrovirus (SMRV), Rous-associated virus (RAV), Fujinami sarcoma virus (FuSV), avian carcinoma virus (MH2), avian encephalomyelitis virus (AEV), Alfa mosaic virus (AMV), avian sarcoma virus CT10, and equine infectious anemia virus (EIAV).

21. The method of claim 20, wherein the pseudotyped viral vector comprises a VSV-G envelope protein,

22. The method of any one of claims 1 to 21, wherein the encoded protein product of the nORF is less than about 100 amino acids.

23. The method of any one of claims 1 to 22, further comprising performing a statistical analysis between the expression of nORF in the mosquito and expression of the nORF in the subject.

24. The method of claim 23, wherein the statistical analysis measures a positive or negative association between the expression of the nORF in the mosquito and expression of the nORF in the subject.

25. The method of any one of claims 1 to 24, wherein the nORF has at least 85%, 90%. 95%, 97%, 98%, or 99% sequence identity to any one of SEQ ID NOs: 1-13 or a portion thereof.

26. The method of claim 25, wherein the nORF comprises the sequence of any one of SEQ ID NOs:
1-13 or a portion thereof.

27. The method of any one of claims 1 to 24, wherein the gene product of the nORF has at least 85%, 90%. 95%, 97%, 98%, or 99% sequence identity to any one of SEQ ID NOs: 14-28.

28. The method of claim 27, wherein the gene product of the nORF comprises the sequence of any one of SEQ ID NOs: 14-28.


SEQUENCE LISTING (28 sequences)

SEQ ID NO: 1 - DNA, 4317 bp, Plasmodium falciparum
atgagtgatattttgtacggcaattctctttcaaatggcaaaaagtccaaaagaagaaaaaaccatattgccaagaatcgagatcagaatgaatcggttcctgtagattcaacatcacaacaatctacaagtgctgataaagaggaaggaaaagttgaattgaaaagtgctgtgaaaactgttccagaaaataaaacaagtaatgaacaaaaggaacaaccagaggaaaattcagctgttgaaaaattaaacacatcattagataatgatgctgataagggagacaagaacaaattagatgttgtagaaggaaccacgaacaatttgcccaaaaaaaagaagagaaacaaaaaaaacaaaaaaaagaataatgaaaataatgaagaagtagctgttaaggaaacgaaaaacgtggaaaacaaggatgtgaaaagttatatatcctatgctgacaaggttactaatggtaaacctgtaaatttaaaaagcggtgcagcagttacgcattgcatactaagagttgatgttcctttaaaacgtggttcgaaaaatgaagaaaggaaatcatcttcatataaatcgtatccaaaatttaataagggaaaaagaaatgacgcttttggaaaaaagtatagtatggggaaagattatttcaaaaaggaaaaattctcaacttttaatcagaaaaaggagagaacattgaattctgttgaaaaaaagggaaaaagttcttatttttatgagaacaaggaaaaaatatcaaattctgatgagaaaacgaaaaaaatatcaaattctcatgagaaaagggaagaaatatcaaattctcatgagaaaagggaagaaatatcaaattctcatgagaaaagggaagaaatatcaaattctcatgagaaaagggaagaaatatcaaatttgtatgagaaaaaaaaggagagtgctccaaaatctgatgagaaaaatgatgaaatattaaatttttatgaacaaatggaagaactattatactttgatgaagatttattagatatattaaattcagatgaagaaaaggaaaataaatgggaggtcgttaaaaaaagagacaaaagaaagagagttgatcataaaaaggaaagtaaatcaagcgaagattatgaaaagtatatattatcaagttctcatgaagatgaagcaaaaacgtggaccattgttcaaaaaagtgatagaaaggtgaaagttgataaggtatctgaaaaaaaaaccacctttaaagaaaaaaacaatttgaatggtaatcagaaaaaggataaaatatccaacgttgacaagcaaaagggaaagacgtcaacgaatgatgaaaaaaaaaaagtgaagtgtgatgagaagatgaaagaaacatctaaggaggatgaaaaacaagtgaaaagtgatgagaacaagaggaaaaaaagaagaaaaccaaagaataaaaagaaaaatttaaatgatcttgagaaagaggaaaaaacgtcacaagttgaagaaaaaggaatgatgataaatgcagaaaaggaaagaaactcaattggtaacaataaaacagtaaaggtagatgaaaaaaaagaaaattcttcaatccataaggaaaaaatagtaaatggtgataataagaaagaaaaatcatcaaatgatgatgacaagaaaaaaaaaccaaatgatgctgaaaagaaaaaaacaccaaacaatgctgacaagaaaaaaacaccaaatgatgctgaaaagaaaaaaacaccaaatgatgctgaaaagaaaaaaacaccaaataatgctgacaagaagaaaacatcaaatggtgctaacaaggaaagaacaccaaataatgctgaaaagaaaaaaaacccaaatgatgctaacacgaaaaaagcatcaaatggtgctaacaaggaaagaacaccaaatggtgctaacaaggaaaaaacaccaaatgatgctgaaaagaaaaaaacaccaaatggtgctaacaaggaaaaaacaccaaatgatgctgaaaagaaaaaaacaccaaatgattctgaaaagaaaaaaacaccaaatgatgctgaaaagaaaaaaaacccaaatgatgctgaaaagaaaaaaaacccaaatgatgctgaaaagaaaaaaaacccaaatgatgctaacacgaaaaaagcatcaaaaggtgctaacaaggaaagaacaccaaatggtgctaacaaggaaaaaacaccaaatggtgctaacaaggaaaaaacaccaaataatgctgaaaagaaaaaaaacccaaatgatgctaacacgaaaaaagcatcaaatggtgctaacaaggaaagaacaccaaataatgctgaaaagaaaaaaaacccaaatgatgctgaaaagaaaaaaaacccaaatgatgctaacacgaaaaaagcatcaaatggtgctaacaaggaaagaacaccaaataatgctgaaaagaaaaaaaacccaaatgatgctaacacgaaaaaagcatcaaatggtgctaacaaggaaagaacaccaaatgatgctgaaaagaaaaaaacaccaaatgatgctgaaaagaaaaaaaacccaaatgatgctgaaaagaaaaaaaacccaaatgatgctgaaaagaaaaaaaacccaaatgatgctaacacgaaaaaagcatcaaatggtgctaacaaggaaagaacaccaaatgatgctgaaaagaaaaaaacatcaaatgatgctgaaaagaaaaaaacattaaatgataaagaaaaaactgttaattttaatcagaaaaaagacgacaatttaaatgttgataataagaaagaaaacacagtaaatgacgctgagaaaggaagaacattaaatgataaagaaaaaactgctaattgtaatcaaaaaaaagaagacaactttaatgttgataataagaaagaaaacacatcaaatgatgatgagaaggaaagaatatctaatgctgatgaaaatatgaaggatgagaataaagaagcagaaaaaaagaaaaagaaaagaaaaaagaaaagaaaatctaaaaaatcagacaataaggaaaatgaagttacattagtatcaaatgatgtaaaatctgatcaattagttcaagaggataagaacgaaatacaaaatcctttagtagataatgaagctgctgattcaactgctgatcaaaatccagacaaaaaagtatttgtcataacatattcatggaaaaatttaaaggacgtaaatgataaggctatgaaattaaatcttgatgatcaaatgaaagaatcaaactatacaaataataataagaatgataaaggagtccttttaaatttgttcggtaaaccttattcactacgccaaagaaaacagaaaatagaatctgttgaagatttggcgaaagaattagagaaaaaaaaaataataaaagaaaatcaaaagaagaaagacaaggataacaaaagaaaaagaaaatacaataaaaaagaaagaatacataataataaaaataatagcaataatggaaatgaaaatggtaataacaataatggaaatggaaatggaaatggtaatggtaattgtaatggaaatggtaatggaaatggtaatggtaatggtaatggtaattgtaatggaaatggtaatggtaatggtaatggtaatggtaatggaaatggtaatggtaatggtaatggtaattgtaatggaaatggtaatggaaatggtaatggaaatggtaatggtaatggtaattgtaatggaaatggtaatggaaatggaaatggtaatggaaatggaaatggtaatggaaatggtaatggaaatggtaatggaaatggtaatggtaattgtaatggaaatggtaatggaaatggaaatggtaatggaaatggaaatggtaatggtaatggaaatggaaatggtaatggaaatggaaatgttaatggtaatggaaatggtaatggaaatggtaatggtaatggtaattgtaatggtaattgtaatggaaatggtaatggaaatggaaatggaaatggtaatggaaatggtaatggaaatggtaatggaaataatgaaaatggtaatggtaatggaaatggtaatggtaatggaaataatgaaaatggtaatgacaataatggagatagtaataaacacatattgcagaatggggtggacaaaattaacagtactgaaaatgaaatacctattcataataatagtgataatataaataatatcacaaataatacgaattcaaatgtaacatgtgaggagaataaggaattaaataaagatgtaaatactaatgaagaaacgagtcagatacagtttcaaaac

SEQ ID NO: 2 - DNA, 2377 bp, Plasmodium falciparum
attattttatgaatgttgtattaatatgtttatttatttatttatatatatatatatatatttttttttttttttttttttcttgtgaattttttaaagttggtcatataatacatataatatatataatacatataataaatataatatatataatttatataatacatataaccatttttataatttatttattttttattttttattttttttattttttctttttagttttattattgtaaaatgcgcaaaggaaaatcgccttcgagcttcttttccctacacaagaagtacctcttgacaggcgtaacgatattatcatttatttttatgtttcagtataaatatcacgaagtattaacagtatatgaagataagacgaatgttcagcagagtagtcgtttgttttggactcgtttattatttgggagaacacgtagtaggataacggggagaatttttaatattgagatacctcactcttatagattatatgtatataatttttttataaaatatttaaatataaataaagaagaaataaaatatcctatagaatcatataaatccttgggtgattttttttcgagatatattagagaggacacgagacctataggcgatttgaatgagtattctatagttagtccatgtgatagtgaaattgttgattttggtgagttgacatcaaattatttggataatgtgaaaggtataaaatttaatataaaaacattcttaggttcggatatgataaagaaatataatgatgatagcacaagtttttattatgctatattttatttgagtccaaagaaatatcatcattttcatgctccatttaattttaaatataaaataagaagacatatatctggagaggtttttcctgtcttccaaggtatgtttaaaattataaataatctatttgatataaacgaaagagttatattatcaggagaatggaaaggtggacatgtgtactatgcagccatcagtgcatataatgttggaaatattaaaattgttaatgatgaggatttattaaccaacaatctaagaacacaattaagttatatgggaggagatattaataccaaaatttatgatcattataaagatctagaaattggtgatgaagttggtgagtttaaagtaggctcatccataattgttatttttgaaaataaaaaaaattttaaatggaatgtaaaaccgaatcaacaaatatccgtgggtgaaagaattggtggtgtggaccaaccaaagcagcccaaaaataagtttatcaaaataaggagttaagcaaaggatatatatatatatatatatacatacaatttatagaaatattacaaagggaaaaatgaattgtatataataagtattaacccactatacaatataataggtagtgaggatatatcatcaccatttggttaaaaaataatatgtaaatggataaataaaaagatatatattatatgtctatgtattaatatatttattaatttatttataaatttattaatatatttattaatttatttataaatttattaatatatttattaatttattaatttatttattaatttattaatttatttattaatttatttataaatttattaatatatttattaatttattaatatatttattaatatatttattaatttatttattaatttatttattaatttatgtgcttattaatccatatatattattttattttatttttattttttttttttttttatcctttgtttgtaaaagaacactatctatatcaattacgtacaaattgtacctctgctttttgaaagggcagggaaagtaaaaaaaaaaaaaaaaaaaaaaaaaaaataaagaataaatagaaataaaccacagcaaggtgaaaatataaatatataaaaaaatatatatgtgtgtatttttttaactcttataattgagtatatgttacatgttctcatgacataatattgttattttgtttttaaaataaaataaaatggtatcgaaccatgggtatgcttccctcttaggggtgaggtggggtttaagcgaaatgataatcctgtttattatacatataataggcgaattataaatgataagaaaaaaaatatatatatgtatatatatatatataaatatatatatatatatttatttattatatatatatttttttttttttattttcattatgagcacatgaaccaaaaacaatatggcatttattaaaacgttgtgtgtgtgttttgtatatgtggtatgtacaaaaaaaaaaaatataaagataaataaaaataaagatataaaatatattctatatattataatttatatttaaaaaaaa

SEQ ID NO: 3 - DNA, 5099 bp, Plasmodium falciparum
atgaacatacacgaaatagatgaaaaaattattactcatggtaatttcatgaggcaagccttttattcaagtaacataaaaataaagactcatgatatcgtacaagctatcgaagacgaagaagaaaaagaaaagaagaaaaagaaaaagagtgataataaagatttattacgactaagtgaaaatatcatttctaaaataaaaagtgatggtagcgatataataaaaaaagaaggttccatattagacgtcaattcacgtaatgtttgttatgctttaaaaaatgggaaatttcgtttaataaatcagaatggtataaatactacaagagtaaaattattatatgataatgaagttttatatgtttgttttaataaagaaaatgggaattatttattattattagataataaaggacatttatatatttataaaattaatgaatataaattagatttaatcttatgcttaaatttccctccttatgaaaaaaaaaatccaaattataatattttaaaagatataaaaaatgttttaaaaagtggaattccaaaaaaagctagctggctacctaaaagtgataagtatttcctgactggtcataataattgtatatatatatggaacttatctttattaacaaatacaataattatgaataagttaaaagatcaaattgatgttactgataagttaatatcattatgtgctattactttgtcatttgaacaagtgatgtataaatataattatatatttaatgaagaaaaaaatgaagtggaaggaaaaaataacttggaaggaaaaaataatgtggaaggaaaaaataatgtggaaggaaaaaatgacgtggaaggaaaaaatgacgtggaggaaaaattaaatatggaagaagaacaattttgtaaaagaagaatatttttgaatacatatagtttaagtttaaatggaaaatatttatgtgcgttaattaacaattattatatgttaatatggtgtgttcatagaaataatcattttttaaaattgacgttggttggtttaaacaatttaagtgtagaattacaaaaggtacataaagaagtgtatgtaggaagtgaaatagcagaaggagaaaaagaggaaggtgataatataaatgttaaggataataacaaaaaagataatattttagcatataataataaaaataatgaacataataatgagaatttaaaaaatgataacttatatataaattcgatccatataataaacaattttttttcacctaatgaaaataataataatgaatcatcgtattatttattagtatttcataaaaattgttgtatatcagtttttaattttaaatattatgtgggtgaattttgtgataatttatcagagcgcattgacatattgaatataaatagtatacagaatatatatgtagagaaaatgggtcatattgaaaatattgaattattcgtagatccatcagaattttttgtattttttaatatttcatataaactcattactaatgaaagaaaaaaaaaaacagaagttaataaaagtcttctgtttgttttggaaatattcaataagaaaaggttagattttaaaatacatcccaaattttttcatataccgaataaatcaatactccccttgtgttcgataagactattacaaataaatgacgcaatgaaatttacaaaagtgttgaatgtatcggtatcatcatcttctttaaatataattaatctatttatgttttctatcatatttaattcttcaaaaaataatgtgagtgtgcaatcgttttcggctccaatacctttgttaatgggcgatgtgtgtggtacggagaaggacggtgatgagatgacacatggtaatgttctccatgctgaaaataataatgatgtaaatggtgataatggtgataatggtgataatgatgatgataatggtgataatgatgataataatgatgataataatgatgaagataataatgatgataataatgatgaagataataataatgatgataataatgatgaagataataataatgatgaagataataataatgatgataataataataatgatgataataataatgatggaaataatgtgagtacctatcataaagaccattgtgctaagagtaccctcgagaatgaaaaatatacctttgatgaatgtacaagtataaacaaagaaattgatgatataaatcatcattacaattttgtaaagaaaaactcattgagtgaaccttatgaaaatattgtaaatattttgaagtataaacaaagtgaagatatggaaaaaatacctaacatgaatgataatgtgaataataatgtgaatgataatgtgaataataatgtgaatgataatgtgaataataatgtgaataataatgtgaatgataatgtgaataataatgtgaatgataatgtgaataataatgtgaatgataatgtgaataataatgcgaatgataatgttcatgttaataatattgctcatatgaatatgaacacttttagttgtgagaacccgcaatccatggaacatataaataattataatataccatcagaagatataaatgaaaatggaggttttgaatatttaaagaaatatataaataatgaagaaaataatttaattatgagaaataaattaaaagaattgttacataagaggaatgagtattatttagggaatgttaaaaatgatgattatgtatctgtttcaacattaggaatatctaatgatgtggatatattaaataaacaaaatagtgatcaaattaattataaatatgatatgaatgaatgtaataaatccttagatcatacatcttctgttgaaaaaaaaaaaaaaaatataaattgtgatgatacatatgttaatataaataatgataatttgttgagctattctataggcgaaataaaaacaagtgggttatcgtttgatgaggttttatttagtcatgatataaaggacaaggaagaaaaagaaaatatttatatgaatgataataatatattgaacaagatgaatagaaataatatgatgaggcacataaaggataataatcaaaatgacgaggaacaaaattgttttacagcaaatatgggaacctcaaatggtaatacgaatggtaacataattgataatataaatgataatataaacgataatataaatgataatataaatgataattttaatgataatataaatgataatataaatgacaatataaatgacaatataaacaataatatgagtaataatagcattagtggggtgagttccgaattttgggaaaaattcaatgaacaatatccgatatgtgtaaaaaagagaagcagcaaaatgagaccctttaacgataccaaagatattgttgacttaatattttctagcagtacaaatggtaagattgaagaggaagaggaaaaaataggagatgaagaaatgaaaagttgtgaggtaataaaaaatgtggaggaggaaaaaataaaaaatgaggtaataaaaaatgtggaggaggaaaaaataaaaaatgaggaaatgacaaatgtggaggaggaaaaaataaaaaatgaggaaatgacaaatgtggaggaggaagatataaaaaaaaaccttgatcataataaaaaaatagatatcattatgtcgaatcatgaagatacgttaaagaaatatatggaatgtgaagaagaagatgtcgaagaagacaacaaatataatgatgataatatgtgtgacaagaaagtttctattttaagcttatcagaaaatgagaaattactggaagagaaaaaaaaggaaagttttattacaaacatattattaaataataaatatgataaaaataatttgagtgatctaacatataataattacaatgttcatactattcttcaaaataatcaaatgttgagaaatggggaaggtaataaaattttccatcatgtaagtgaactagaaggagaagaacatgtacaaaataaagatgatattgtaataaaaacaaatataaatatggatgatacacatatgaatataaaaaaagatgaaacatttgggaagaaagaaaaaaatttaaattcatttttacaaaaattaggcttctttaaaaattcatatcctgtaacggtagactccctattgaacaacagtgatattattaaaaatgagatgcataaggaaacatcttcaataaaaaaaaaatgttctgatatgttagatgaggatactacatctcatttgttattatttaacaggatgagtgaaaaatatatgtgtgataaagaaatggagaatgaaaatgtgtatgcatttttgcataataatgtgcatgataacaacaatatggtggatgataacaacaatatggtggatgatagcaacaatatggtggatgataacaacaatatggtggatgataacaacaatatggtggatgataacaacaatatggtggatgataacaacaatatggtggatgataacaacaatatggtggatgataacaacaatatggtggatgataacaacaatatggtggatgataacaacaatatggtggataatcacaatactatgcgtagcgaagatgaaataaaaaatatggataacatattaaatcatggtgatgataagagagaggatgatgtagattcacatgatagagagggctcgttaaataaaaaggacatctttgaaaaagtatatagaaatataaatagaaaggtatcagaaataatatatgaagagttgagaaatataaattgtgtagaaaataacgaaataacaaatgaaaaaagttatagaaaaggtaaaggattaataaatattgataaatataataaagaaggagaaattcatgatatgaataaaagtatatttaatgaacatgataatagattgattgaatatagctgttatcgtaatagtattgaattagagaaagaagtaaataaatatcataagaaaataaatgaactgaatgaggaaattatacatatgaagaatatgcaagagaatttaaatataaatatgataagtataaataa

SEQ ID NO: 4 - DNA, 2604 bp, Plasmodium falciparum
ttttttttctgttttttctgttttttctgttttttctgttttttctgttttttctgtttttctttttttttttttttttttttttttttgtaagatggatgatagtgaagatgttgtaggagatgatgagtacctagatgaattttctgatgaaggagattcgaacgaatttcttagtgacgttgagaaaaatagtaacaactcattggatgataaatccaaaggaggaaagtgtttggatgaagaaagcatttgtgaagataagataaaatatgataagagtagtatatataatagttataataatagttgtgatatatttaatttgaaggaagaggttttaggtattcataaaaagaatatttgttgtataaaattaataagaaacggtagtaacttaataatatcaggaaatgataataatgtaagaatctatgaatttaagaatatgaacagatatgaaaagaattatacaaaattagtaagtctaaaagaaggatctattattcaatcattagatgctagagataattatattttgatagcacatggaaataaatgttttgtatataataaaaatggggaatatataaaaaatagtatacgaggtgatatgtatattaaagatgtaaataaaacaaaaggacatactagacaaattaattgttgtagatttcatcctttaaatgaacaagtttttataagcggtagtctagatagtaccataaggatatgggacatggaaaaggaaaataatacatatggattaaataaagaattgattcattcccaatgtataaaagtagttaatgagaaaaatttattaacaaataatattatatcatgcgaatttacaaaagatgctaattcaattattatcggttgttctaacgggttgattgatataagaaataagttatcaaatgattatacatataattataaatcaagtgataattattatataaaaaaagatgtttgtcataaagattcaataatcgatatattaacttgtaaaaaaaataaaaattatttttttacacgtagtatggataataccatcaaatattgggacctacgaaatcttcatttatgtcttcatacgattcaagatgtacctaccatatatgaaaaaagtaatatgtcatattttcataatgaaaaatatttaattataggtacccaacaaaaaaaaaaaaatgattctattcttttagatgaacatgtaaattatatatcaagtaatgtaaataataagaacaatactcatgatataaaatatcgtcaaaaagttgaagattttgaaaattcacaaaatgattatgactgtacagaagaaacagatataagaaaaaacaaatataaaaataaggaagaggaagaggaagaggaagaaaaaaaaaaaaaaaaaaaaaaatgtattattaataataattatattaaagaatataaaggtgatgaagatatgaataattttttaacagaaatgtcatcattaaataaaatagaaaaaaatatatttggaagtatacagatatatgatatagaaaacaattttaatttagtatatactaaaaattatgaatgttcaggtattatatgtacatattatgatgaatatataaaacaattatttttaggaacaaccgacggaagatgtcttatatattatggtgataattcaaagaaaggtgtattagattatttacaacaaaaaggttataaaagaaaagatgaaacaaatgaaaacacgtttttctatatgaagaatgataatatatataatttagataatttaccaaaagaaatacaaataacgcaatcgggaaatgtaataattaagaaaaataatctaaataataaaaaaataaaagtaaatccaagtgttcaatcattatatcaaaatgcatatgaaagaaaaactaatgttagtgcatatgctaaatttattacagatgataataataacaatttaaattatcatgatgatataaaaaatatttcaaatataaaagatgataatatagttgaagttttaagaaatagagaattaaataaaaaaggagatgactactttatgaaggcttataaatatacacagccaaataaaattattgattattcatcatcagatgaacaagaatattccaatattttaaaaagaccaaaatgtcctcaatgtggaattaaaaattgtgtttgtggttatatgaaaaataaataaatatataaataaatatataaataaataattgaagccacattagaatatctacacgtaatataatatatatatatatatatatatatgtataaatatacattttttatagatgaacatattattatattaaaaaatgtcatcattttttaattcaaacatttcatttttataagtttctttttattttttttttaaacaaaaaatataaacattttacaaaaaaaaaaaaaaaaaaaaaaaaaaatacacataaataaataaatatatatatatatatatatatatatatatatttatatgtgtgtgtttttttataggaataaaaataaggataaaatatgttgccgtacatattcatatacttaacacatattttatgg

SEQ ID NO: 5 - DNA, 2604 bp, Plasmodium falciparum; identical to SEQ ID NO: 4

SEQ ID NO: 6 - DNA, 3365 bp, Plasmodium falciparum
attaaatgcttaacaaatatgaaaagctgttttgcaggaacgatttattaactatacatatatatatatatatgtagttatatttataatattacaaaatattgtcttttattttattttatttttaacaccgtgtattcctcaaatttcaggacctaagccttttctctgttcttctttattaataaagagaaacttgtttaaacatgcacaaaaaaaaaaaaaaaaaaaaaaaaaaaaaaagacgtagagaaacgaattggcatccttaataaattcgtcaccatatttctcacaataaggatatatatatatatatatatataatgtatttatatactttatatttttttgtttgatatatacccacaacatattatctttaaattaaaaattaaaaaaaaaaggtattaaagccccaaatgattttgtaaccactgggaacaaataaaataaaaaaaaaaaaaaaaaaaaaaaaaaaaaaaacacatttgtcatttatttataaagttatatatatatatatttatatttatatttatttatttatatttacatatttttatttttttgtttgaaaaaaaaatatgggaaacaccttgtccaattcaaccctattccgccctactgaaccaagtgttaaaataaaaataaaaacctacatttataatatatatatatatattgttatatgtccatgttcatattatttatgaataattaaatgaaatttatatacacaatcattagacgaataaatatatatatatatatatatatatatatttgatatattacaaaaaagtaatcatatatatattatatattcattcatgtgtgtgacaccatatttttatcacgtgatatggaattatgataattacatattgtatacatttaaaatatatatttttttccatcttctcagtatgatgatgaattgaaaaatcttgtatacatccccgaattaatgaacattgatgtcaacaaattttggaatgacaaaacatatgaaatattcaataaggaagagaatataaaagaacttcataataaaaagtttccagctttatttttatattatactaaagatgtaaaaactaaacataccataatgtattttcatagtaattcctgtgatctaggacaaatttatgatgaacttaatcatttacatgaacatttgcatgctaatattttggctatagaatatataggatttgggttatgttatttagaaggttcaacaaaccaatataatattaacagaagagcattagctgcttataattttttaaaatcattaaatattaaaaatgaaaatattctattatttgggagatctataggtacaggagttgctagtaagttagcatataacctaaaattaattggggtgtcagtagctggaattattttacattctccatatatatctatagaaaaattagttgaagattattttacttattcttcttattttattgaaaacatttatgataattataaaaacttatccttcctaagtaacaacacagattcagatataccaattttgttaatacatggtaaagaagatgaaattattcatgtctcgcattcagaatatttaatgaagaatttgaacaacaaatttaaacatgcttcttatccaactgattcgtatcataattattactacgttatagatgtaagtcaaaaaaaaaaaaaaaaaaaaaaattacattacctaattgtatatatgtaatacatataaattatatatttatataaaattttttatgcacatttcaatcactactatatatatatatatgttttataaattgtacaggatcttggaattccaataaaaatattcctagaaaccttgagcaaatcaaagaatgcaaaatctgttgatattaatattcccaaagtttatttttatagagagtaatataaatataattttaagaatatatgaatatatatgtatatatattcatatatatatacatgtgcatatatctttttccctttagacttatatattaccgaataaatggcctcgatgaaacattcaaaaggttaaatgaagacgaaaaaaataaaaacacaaatgacaagaagacaacaaaaggagagtcatcaaaagatatatgttccattgataaattaacaaaaaaaagagattccaaaaaaaaagataaaaaagaaaaaagcacaaatataacgtgtactaatataatcaataacaattcaagtaacgacctatctatacaaaatataacaaacgctaatataaaaaataaggaacaagaatatacagatcagagtacacaaaaaaataataatacatgttctaaaaaaggggattcaatatatagtattattaaaaatgaatatataagaaatacacttgaaaacaatttaaaaaaaaattatatggcatccaaaaaacattatcataaaaacgatgaagaaaaaaaaaaatcaaatattaaacaacacaatgtaatacacaaggaagatgaaaataaaattttaaaacaaaaacacagtgatttaaaaaaaaataaacatctagataaatataatatagaagaaaatgttcataaaaaaagtaaagaaacaaaaaatacaaaaacctcaaaaggatatacaaacattttaaataaaaattatgataaagatatcatcaatgaagaaaaaaaaaaaaaaaaaaaaaagaatataaataatgtaaatataaatataattacatcatcatcttcaaaaaaaaattttaatgatcattatgaaaataatttatttgataaatgggatggtaaaatatattgtaccaagaaaaaaaaaacaacagaagaatctgatgaaaaaaatgaatttaatacaaatgttgaacaaaatatttcttcctatcaaagtaaccaaagaaataaaaaaggaactaatgatgataataacttggataatgaaaacaatatatatataaaagaatgtaatatttatgatattaatagtataaataataataataacaagtacgaaatatgtaattatgataatgataatgataatacatttaaaagaaactcatcatcaagtaatctagacaaggacaaaaatcataccttatataatgaaaaatataaagatgaatatcccaaacatgtacacgaaaaaaataaaaagtttcctattattcattataaaataaatgatttcaaatattatatcaaaaataatattattaataaagaaaaaattgataatattgttgaagggtaatcaataacttaaatgaaaaagaataattttgtgattaaatatatatatatatatatatatatatataataggagtaaaaatatatataatca

SEQ ID NO: 7 - DNA, 3011 bp, Plasmodium falciparum
attttttatttatatttttataataattttatcttgataataaatataaaatgaagaaagatgaaaaaaacaatatggacgaatcattagaagaatatgacgaggaaaactatacaagtgaacttgatgatttaccggaatatgaaagagaactcatattagctaaacgccatgaagaatatatgaagaaaaaacatagacgtatgttattaaaaaatttacatattgatgaaaagtccaaagggaatattcatgatgatacaaataaaagtattgaaactagtaataaaagaaaaggaaaattaaataataaaaaattgaagttaaaaaagaaattatatgaaagtgatgaagaagaagaaaatgataaaaatgataaaaatgatgatgatcaatatgataatacatatcaaagtgatgaggaccggaaaaaaagtaataataaaaaaaaagaacaaagtaaaaaaattaaaggagataatgaatcatataatatagattccatttcgtacgtgaaatataaaaaaggaaaaataaaaatattagaagatgatgatgaaaagaaccatgaggatgaaaaaaaaaaaactgatataattgatcatgtaagtgtgacagatgattcttatacagactcttcttataaaaataagaagaaaagtaaatataagaagggagacacaaatcaaacagacaaaacaagtaaaaaaaataaaaatgataaaacaaataaaacaaataaaactaatgtttctataaaaacgaaaaagcaccgtgaagattattcagatgattctatggatgattatcatagtacttcatcttatgaacatgaaaaaagtagtgatgtacattataataaaaaggaaaaaaaaatacagttaaaagtaatttctgaagagagtcatttagataatactaagaataaaactggaaaaggtttaaacaaaattttaattgaaaaagaaaaaaaaaaaaaaaaaaaagatttacatatcgttgatagtgttcataagaataaagaagacgggaaaaatgttattaccgatatttttgaaaagaatgaaaaaattaataaagataaggaacttgaattaccccaaacacctgaaagattaatacatttatataaagaagaaaagaaaataaaaatggatatatataattatatgacatatgatattattacatatttccaattaaaaaaaacgtttcttcttgatatgtgtgaacatgtacatttttcttatcatgttattggtcatatgataaaaattatagatatctccaagttaaataaagttactaatcaggatgtacaaaatacaaataataaaaaaaaaattgatacagcaaatgaaaataaaaaaaaaatatttttcataactaatgtaataaaatcagaaccatatttttctacagatcgtaatactaatattaaatttgaagtagcacatttagaaaatttagcacattctaaattttttaaaaaaatcaaaaatattatgtatcaaaataaaaaaatatcaatggatgaaataaaatcaaatgaatattatgaaacatatatttgtgatatgaataatataagtgatcaaaaatttacaatagacgaatataatcatataaaattattttctatagatttagacgtattaaaaacgttccatctatttttaaaagaaaaaaatgaagacctaaaaaattttcgttatacagagaagcagttacaagaccttttcgaaaagaagaaacaaaaaagtttttacgaaatttatacaaataataaaagtatagataatttgccaataactcgtattacagtacaaagggaaatttgttctatacagagagaaattgataaattgaatttcgacaaaaaaaaaacaaatgtaaatgatacatatacattgtcgaagttaaataatcaaataaatgaattgaccaaaaaaattaatattttacgaggaaatctggataaagctcgaagtaattttttttttcttcttttctaaaagagattaagttaatacttgatatatatatatatatatatatattatattgaattatttttttattttttattttttattttttattttttattcttttatttttttttctgtagaaaaccatgctgcaatgaagtctaacgatcaattcgctcaagaaaaaaatatgactaaagaaaaatcaaggactaacataaaatctacacagaaggatgatgctacaaataaattcttaggtgatgaattgtataaggatatataaataaaatatgtattattattaacaagagaaaattttgtctttgcgaaaattataagcaaacaaatataaataataaaagaaaatatataaattaataatatatatactagccatatttttttatatatatacattaatattatcaccttataccatatgtataaaaaatttatgaattgaaattcttttttttttaggagtgaaagaaatggatatatcgtatttctcgaaacaaatatatgatgagaaaacaaattttaataatatgctttcttcaaaatttataaatttaccacttgatgtacatcacaaagttgttcatcactttttattggggaccatacaaaataatgatgcggtaaaaaaattaaacgaaacgaaataaaaaaaagtctatatatatatatatatatatatttctatgtgtttataatatttttaggttttttatgatgacccttctcacaaaattaaaaaggaattggatgcagaaattgataaactccttggacgtaataattaaaaaacaaaaatgcatacaccatatcttataatattcatatctcatatatatttttatatatatcattatatatattttgttttttgtagttcatgggaataggcatataaccctgtttgatgatataaaaaaaaattacgaataagataaaaatgagaattaaatggaaaaatattagacaaaaaaaaaaaaaaaaaatttatatatatat

SEQ ID NO: 8 - DNA, 2990 bp, Plasmodium falciparum
gaccgatataacaaatatcccaaaagtacacatataaaaaaaaaaaaaatatatatatatatatatggacacttgtatgtctcattagaaatatatatataatatagatatatttttatttttgatatattattttgtaaccttataatttcacaaaaatatttaattgtttatgtactaaaatattatctatctctctctatatatatatatataaacgtgatacatatagaaacaaaaaaaaaacaaaaaaaaaaagttatatctttgaattataaaaaaattaaatcaaaacataaggacaacattttaacaatttaaaaaaataataattataaataaataaatgcataccacatacatattatataaacataaaaacgaattgccaacatccttttccctgaataatacattaaattaaaaaagcatatttccttctagaatatattttattaattttatcaaaaaaataaataaataaaataaatacatattaaataataatataacaaaaaaatcattttagatattaatttcgatttttcttttaacatatttgtatatttaaaaaaaaaaaaaaaaaaaaaaaaacgcctatttattttcttttaaatatacttcttgattttttcattttgaaattaatatttaatacttaaaatgttatatatataaaaatatatatatataatatatatattatatgtacattattgaaatatatactaattatacataatatataaatattatatatatatatatttttttatatgtaatcatataaaagaataggccttgaattttatactgaaaaaaattattattattatatatatttacatatatatataataatataatatataaaaatacaatatgaaaaaaaagaaaaaaaaaaaaaaaaaagaaaaaaaaaaaaaaaaaaagaaaaaaaaaaaaaaaaaaaaaaaaaaaaaaaaaaaaaaagaaaaaaaaaaagagcacatatattttattaatagttattataacgaaataataataatatataattttttttatttttaataattttccaaatttttttaatttttttataaatcttttatattttatatatattatatttcttttatattatatattttattacttattaatattatataatatatatatatatatataaatatttatatgttatattattttatttttttttgcattcatatattattattattatatttttatacctttaattaaaaatgatataaatttaataactgttaataagtaaaagtaaaacataataacaaaataaaaatataatatatgtatatagtaaatatatataatattatatatatacacatatagtaataataataatatatatatatatatatatatatatatttatttatttctatcctgatatatatatattttttttttatccacccagttttattttgatatacaaaatatgttcttctctttcgagaaatagtacgatttttttttttttttttttgattaattatatatatatatatatatattttttttttttttattttatttattttattattttggtaattattacctggtttaaaactggaatgaataaatatcattaaaataagaaagatggcattagtaagatgaaagaagataaaagaaaacatatttatatgtagaaatgtgtatataaaaatatttacctatttgttaatcattttagtagtattttaacttgaacaaataatatacatatatatatatatatatatatttattcatttatttatttatcttaaccctttcaatgttgtttatttgccacaataacactttatgcatctctccttatttaagaaaataaaatgaattaatagaacaagaaaataactttctccgtttttatatatgaacctaatatagttttgtataacaaatatatttatattacacatacgtgcatatacatatatatatatttatatatatatattttttttttttatcattgatcccgtttttaggatgatgcagaagctcaaaaacaaatccaacaaatggtaaacttcatcttgaatgaagccaaggataaagcacatgaaattgaagccaaagccttagaagattttaatattgaaaaattaagaattgtgcaaaagatgaaagaaaagatacgtgtagaatttcagaaaaaagctaaacaaatggaaattaaaagatcgattgctagatcatcagctataaataaagctagattaaagaagatgtgtgcaaaagatcaagtttttaaagaaatttataaaattagtagtgataaattaaatgatttatataaagataaagataaatataaaaaccttattgtagatttaatagttcaatcattattctatatgcaagaaccacatgttattgttcgatgtagagatatagacaaagcagttgttgaaagctccttaaacgaagctgtttcaaaatatacagacaaattaaaaaaacaattcaatgttaccaaaacagttaaaatagaattagataaatcgggaaattatctacctccaccacctacacctgaaaatgaaggaaattcatgtcttggaggtgttatcttaactacaccaaatagaaaaattaattgtgataatactttggatgtgcgtctcaaactagccatcgaatattgtacaccggaaattaaaagaatgttttttgaaaacgcataaaaatataaaatatataaaaatatatgagaatattaaaatgaacacttttatgtaagccaatatcccaagtgtatcaaaaagaataaaataaaataaaataaaataaaataaatatatatatatatatatatatatatatatatatattttagaaattataacaattttcccattttttattacaacagaatattctttaatgtgcatataaatttatatgtatgtgtatgtatttatatatacattattattact

SEQ ID NO: 9 - DNA, 15376 bp, Plasmodium falciparum
aaagtaaataaaattaaaaaaaaaaaaaaaaaaagaaagcaatatagtaatataacttttatatgtttaattaagaaaggaaaaaattataaacgaggcaaaagaagcaaaaagaaaaaaaaaaaaaagagagagagaaaaaaaaaaagtagagggaaaaagaaaagtatactgaagtgaagatatgtttattaattcaatataaaaggtatttcaataaagagcagaacaaaaatatatagtggtttaagaagaaaaaaaaaaaaaaaaaaaaaaaaaaaagacgaaataaattgaaaagaagtattacagaaaatatttagttgtataatagacaaaatgaactcaaataaaaattttgttaataaattatatagatattataggaaaaatgataactataataagaaaagaatattcatatatgaaaacaccgtttattatactcctgatgattgtgtattttgtacacaattaaattgtaccattaaaaagagatgggataaaaagaagaaaaaaaaattaaaatcagctgaaaaggaagaatattttaaagattataaatacttcgaaacggagaataatgataatactgatgaatattataaaaatgatgaagaagaaaatgaagcttccgatttaatgataacatattgtgataggcatgtaagccttataaatattttagttaatatgtttaagatatataataaaaaaaaaacatatttaaatgttacgttaaacataaattacgttttccttcaaattatatacaatattattttatattttgtttacttatatatatattataccaaatatatatattatatattaagaacatatttgttcctattattatataatagttttcatatcatacgagaaaattgttattatccgaatatctttttgtataattattatagaaacagaataaaaaagaatattctacaacaatggaaaatgaaaaaaaagaaaaagaagaaaaaaagaaaaaaaaaaaaacattctcttttcctatgtaagaaaaatatgagggacacaaagagtaatcattttgttagaaactattcatataaaattataaaggattcatataccaatcacgttgatgatgttataattaaatttttattatatatgttttatgaaaaattcataaataaatttaataaatatattaatatacatcatcattatcataatgtttataaaaaaatatattattataagataaaagaaggaatgcttccatttttgtgtgatcatatattcaagtgtaattacaatttattttatatctttaatagatacaatttccaatttaatgcatacaacatattattaaattgtaaagaaaagaacagtgaacaaatatttatattttttaacaagaaatttttaattaaaaaagattatgttatgaactaccttatttatgtaattaataatattagggaaaaaataaataaaataaaaataatatatgatgatacaaaaaaatgttattttatattttataaaaacaaatgtacctcttgtcttataacacataatattttaaatgtaccaattgaaggtattaaaaaaaaaagtgataatatttattttgagaaaccaaaaactaatataaaaaatgaggatgtcaattgtaagtataataaaatattttataaatattacataagtgtgataaatgatatgttatgtatatcaaaaaataatatgattattaaaaatattgatagtataaataaaaatcataaaaggctaataaaaatatctaattataatataacaaatgtttgtatgtttttattattacctgagattttttgtaaatattataaaaaaggagataaaaaaaagaagaaaaacaaaaatgtaaaaaaaataatatttaaagagagaagagtgaatattaagtcgtataagatattattaaaatgtgaaatcctaacacattttaatttttttaatgtaattaattgtgatagatatatctatcaaaataatttgtataataatctagttgggttaaaaaaaggatcgtacatgaaaacatgtacaaatgatgagacatatagaaatgatggagatgtaaataataaaaatggttataataataatgaacatagtaatagtagtaatgagagaagtaataacaatggtgataataataataataatcataataataataatcataataataatcatcataataacggatcaaataatcataatggtaataataatagtaataatgatgataatgataaggatgacgataaaaaaggaaatgataaaaaaaatgaagacaaagacgatgaaaataaaagtgacgatgataatgatgatgataaaaaaaatggtgatggtttgaatcgttccttggataccaagaaaaggaaagaaaagagtaagaacaagaataaaatgaaaccagataacacattaaatgaaaatgtaaaagaattattatttgataaagaaggaaaaagttatacatgtactaataatgatttaagtctagtaaataatacaatatgtgaatcaattaaaaagagaaaaagtaatagaggagaaaagagaaattataataatgaaaaaaatgataagaattcattaaataatgttatagctgaaaatgcatataaaattataaaaaatggtagattatataaacctactgaaccagttaatatatttcgatcacataataaatttatgttaaaagaaatgatgtggatgagtatagattattatgaagaaaagagatggaaaaaaaatgtttcaaaaaaatttggatatttaatgagtaaccattttgaagaaaaaaaaaaaaatgataaatattttatttcttctcaaatatcaaatgatattaaaatgttttggttttttatgttaaatgaaattagaccagatttagttccagtagatttagatcataagataaaaaataaaaatcacttaaagaacgattttttgaattcgtttagaaaaaatctacaaatagaggaatataatcttgagatggatgataataatcaatattcctttaatcaacatgatggaataaatgaggaatcatctgtacaaaataagagtgagaagaatagtttctctataaatacatctattgatgataattggggattaatgaataagatgagtcatcaaaatggaaaaaataataaccataatgattataataacaaatgtgaagataataaaaataatgattataataacaacaaatgtgatgataataacaataatgattataataacaacaaatgtgatgataataaaaataatgattataataacaaatgtgaagataataaaaataatgattataataacaacaaatgtgatgataataacaatgatgattataataacaaatgtgatgataataaccataatgattataataacaaatgtgatgttaataagcttaataatatttcaatagaacatactaattgtattaatagtaataaatataatttgttaaacatttccacacataaagaaataaatatgatcgataataatactaatgatattagtacggtttgttgtgaaaataataataataataataataataataataataatgaaaggaataaccatataaatgtaatgaaaaaaaagaatatcatagattatgaaaattatgatcaaattgttatgaatatagaagaaaatgttaataaagaattattgaaaaaacataaaatatataaaaaggattattatactgttgaaaatgttcatataaaccatatacaatatgatgataataatttatattataatgatgatctatataattattatcaatctaaaaatgatgatggatatttagatttaagtaatcatttatatatttattgtttactttttataagtatttctcttatcgaaattaatgaacatattaatttttctagacccgataatcaccttcctaatgatgtagataacgcattgttagaagatgaacaaaataatagtaataataataattataataattataataatgatatttttgttaattatatgaatgcatataataatggagaattattttgtaataatacacaaagagataatattaaatttccacaatatggtacatttaaaaaaataaaaaacgaatttgaagatttcagagaattgaaaaagaaaaagaaaacagatatgttgaaaaataacaaagatggtttattaagtaatgtagataacattaatttagatatcataagaaaaggtaatactgaatttaaagaaaacaaaattgttttgaataatgaaaataatgtatgctatccgaatgcaaaaagaaatagcattatggataaagatatatatgaaaatgaaaaagacattaataattgtgaaaataatatattaagaggaaaacaagaaaacataaaaaattatgaagaaaacaaaatgatagaaacgtattatgatgaaatgttattaaagaagaagagtataaaccaattacgattaatatcagaagagatgttattatccaaaagggagaatgataatggtaatgattataagaataaaaattttatagatcattatgatgaagaaaattataatggatatgataaattaaataaatatgtaaataaaagaatattatccattgataataataataataataataatattgaaaataaattggattcatttgttgtacataaaaatatgaacaatagtaatatttataatattctcagtgtaccttcattgaataacacagatgtaaaagacctagtagttataccctattcatgtgataataataataataataataatagtaataataataatagtaatagtaataataatagtaatagtaatataaaaacgaaagaatcagttagtactactaatcataataatagtagtaataatataatatatgaaaatattaaaaacgattatattaatgaaataaaagaaaaaaatgtaaatgtttatttacacaatatggagttgtatggaggcataccatatccctttgaatataaatatattgatgatttttatgatgacattaatttgcctttaatgaatttagctgaatatgataagtacacttttttcttatatttattttatatgaaaaattcttcttatatattaaagaaacaaatgaaaaaggataaaagaaaacgtaagaaagaattcacagatgtacttttaacaaaaaaaagaacacctaagaggaaaataaacaaattagaaaatgaaaagaaaatagaacaaatagaagatgctgaaaaggttttaagttcagaaaaagtagttgatggagaaaatatattaaaagttataaatgatcaaaataatttatcaaatgaagttaatattattaaagacgtaactatttcaggtacaaaaattaatagcaaaaataatgacaaaagtgatgacaaaaatgatgacaaaaatgataaaaaaaatgatggaaaaaatgacaaaaatgatgaaaaggatgataacaaaactggcgaaaaaggtgataacaaaattggtgaaaaagatgataacaaaattaacgaaaaagatgataacaaaattaatgataaagaaatcgttggtactgttttaaggacaagtactaagaataagactaaaaataaattgaaaaataaagatggttatgttattaaaaaagaacatttaggagatttagaagaacgacaacaaaagaaaattaaaaagaaagaaatggaaactattaaaaaatggaaagagaaaaaagacgaatgggaattagaagaaacaaatttccttcttatattaacaaaaacgtacatgaattatattaacatggatgaaataattaagagggaatatggaatatcattaaatagctgcacagcattaggagacataaaaaataagaataatatgaataatatgaataacatgaataatatgaataatatgaataacatgaataatatgaataatatgaataatatagatgaaacagataatgggaataatataaatattgaaaataatgtatataatagtaataatggcaatattaataatgttgtggaaataaaaaatgcgaataatagtaaaacaaaggaacatgaagaaattaataatgataactcaaatgtagaaaataaaaacgtaaccaatgattttataaaaaaatataaaggacatgaatatatcatgtcttttaataaatttgaaaataatataaataataagttgaagaattataccgattttataaactggaatattatatcggtagctttagtatcatataataaaataagtcatatatatgaaaagaaaagaggaccgaatgaatgtaaagaaaaatttctatcattaataaaagatgatatatataaaattaccaagttacaaaacctgtatcatgattattatttgaataccataaaaaataataatagcatgaaaagaaaaaagaaaaaatgtataaccaaagtaaagttcttgccattattttcatcaaataatgtgaataaattaataggtatgtttaataaatatatgaatgaaaaattaaagaaatatgaattaaaaaaaatggatatgaataataataataaaatcgaaacattaaaagtgaagaatataataataaagaatgaaaaaattgaagaaaaaaagcatgatgaggaaataaatatattaaataaaaatgatgataaaataaatgaatataaagatgagttaaacgttaaagatgaagatattcatttaataaaaaaagtaaaaaaaaaatccagtattataaaaaaaaagaaaaaaggtttttttcataatctttttagtacattttctggaaaaaaaagtcgagaaagctcagaagaagatactgattgtggtagagatagtttttttagttgtagtgatgtagaaatttctgcaacaaaattagatgattcatgcatatcatttagtaataataaaaataaaagtaataaaaagaaaagagcaaagaaaaatataaaacttggtgaagaagaaaatgaaagtgaatgtaacaatgaaggtgaatgtaacaatgaaggtgaatgcaacaatgaaggtgaatgcaacaatgaaggtgaatgcaacaatgaaggtgaatgcaacaatgaaggtgaaggtaacaatgaaagtgaaattgcaagtactaaaaaagaattattagatattaataataaaattaatgatgagaatgataatcataattttaataagaagagaagtagtattagctattattttgatgattattttgatgaacattttgaagattatttagatgattattttgaagatgaatgggatgattatgtggacgaaaattttgttgataactctgatgaattttttgaagaagatgtaaatgaatttaaaaaaataaataatattgatgagaatactactaataataatagtacagatattataaaaaaaaatgagaaaaataatatagaacatataaaagaattgaataatacctatgttgatgaaaattctcacatagaaaaaaaagaaccaaccatttgtgtaaatataaaaaatagtacaaatgaaaataataatatattaataaacaatgatacatttaaatcgaaagatgaaaaaaaagatacatataataaggaagtatgtgtaaatagtatatcacaattaataatatataataaatgtaaagatgatgaatataaatatgatttcaattatattttaaaaaatagagataagataaaaaaatggttgaattgtatatgtaagaataaaaagaatataaaagaatttatgaatataataaataatgattattttgttaaaaataaatattttcatagattgattgaaagtgttgtaaataatgtaagtaaggaacaatcatcatttacatattctaataatataataataaattcattaaataaagaaccagattatgaattaacatatagagaaaatagcaatgatagaaatataaataatcctttagataatgaatttattaaaatgataagaaaaataatggaatatgataaaaagagaaaattaaaaagtatacaaaaattaaaaaatatatcaaataattatagttataatacaattaatgaattttatatatcaaaaccaaatgaatcatataatacagtaaatgaatttgttcagaaaatattgaattatgtaccttatgtagatgataagaaaaaaattattataaaaaaaattaggaaaagatttcaatgtaaaaatcttataacacaaattttattagctgactatatattagataaattaaaaaatttcccaattaatgaatctatagaatcatctaaaaataataataaaacaattgatatgttattatctgaaacacgattaaaagcttatagtaaatatattaatgaacattttaatgataattacgatccagttaaaattgaaacatcatttttaaaaatgttttcgtctgaaaatgatgaattatataaaaagaatatattgaataataataataataaagaagtaacacatataaatattaagtctgaagaagtcgcattaaataaatctatccaagaaacgacaaatactgtattaaaagatactattattgaatcaaatgaaatatcatgcattgttgataaaacggtagatatacataatgaacaaaatgataaaaagcatgacacttcccctgcagatattaataataaggattatattggatatatgcatgttaataagaaagaattggtatctatgcaaaattcttctaatccttcagatataattcagcaatgtgttaataaggttgaagtgtataataaggatgtatgccagaccaatggatacatgaatatcaatgatgataataaaatgaataattttgtaaataataatataactcatatcaataataatgtgaataataataataatattgtgaaccatttaaataataataataataataataatgcgaataatataaataatttgaatgtgaaccatttaaataataatataagccaaattaataataattataataatagtaatatgagtagtatttatatcgcatctgatcaaaatgaacattttaataaaatgacagtgatggaaaaacaggatccaatgaataacacaaaacaacgttcacactcatcatatcacacatcttttcctatgcaaaataataatacgccattatacaataaattggaaggagatcaaattaaattatcaaaatataatagtttagaatctaataagaataatatagatatcatttcaactaataacaatgcaaccaaaacaaaagatcacgtaattaaagcgaataataaaggatcaacaagaggcaagagacaaaccttatcattagtgaaaccacataaggaaagtgaacatgttcaagattcctctgtaataaatcaaccagtaaaaaatgtatcgtccaaaggggaggaaattatattaggtaataataggcagaaatcacaaccaattaaaaatgtagatattacaaataaaatgacaaaatataaaacttctgttagttcatccattttatcttcagataaattaaagaacaacaatattttaaatactgtacaaaatgaaagtgcatatttatccagcaaatttaattccaacgtgcaaacgttaaataataatgtgaataataatgtgaatattaatgtgaataatatgaatagtaatattagtaataatatgaatagtaatataaataataatatgaatagtaatatgtgtaataatatgaatagtaatatgagtaataatatgaatagtaatatgcacgttatggatcaaggctcttttttcacgtccaatatggtaaatagagaagttaataatttattaaatgaggaacaaagaataaatacaaacgttgtaagtagtcattataattctgtagtatcaagtacacaatttataaatgatagaggaaattacacaaacagaatacaaaaaatattaagtaataataaatatagtttaactcctatgaaaaataatataactcaagaaaatatgaataattgtatagttaatcatcaaacaccaaataaaaacatacaagaatattatattccaaatgcatcaccaaataaattaaataaaagcatatcaaatgtagatataatgaaatcaaatacgtcatctatattatatccgcaagcacataattcggtttcgtctaattttaataatgtatccgaaactctacaaaatgtgaatgtattgcatgcttcaaagaaatatcatatgaacaattctaatgatatttgcacaaatataaataacaacaacaacaataataataataataataataataataataataatgtgcatcataacagtatgaatcagaataatatgaatactatcaatatgaataatattaatatgaataatatcaatatgaataatattactatgaataatattaatatgaataatatcaatatgaataatatcaatatgaataatactaataataatgtgaataatattaatatgaataatattaacagtaatgtaaataatattaataattctatgaataatattaatattagtaataatattaattataatattagtaataattataataacagtgtaagtatgtacaatattccaaataaggaagatgataaatttatatcacccttacattcttcaatgcaaaataataaatatatgcctataaatcgtaataatagtagtatgtcgtataataaaacacctatatgtgatagtgttgaattaccaataaaacaaatgtcaggaaatatttcaaataattgtaatgcttctatgcaattagaagtttcatccaatagttcatcaaagaaattacaacataccataggtagtaatacaatcaatacagataaaaaatccttattaatgtatatagaaaatagagcaagaactggatcatcatctataaataaaataaatgatgatatgttctctataaaatcatcaactccaagtaattatcctccgtcctatacaacacatcaacaatttatatcagaaagtgaaaacaagtcaaatttatatcatgaccaaatgttaaatcaagatgttattatggataataaaaatgtacaaaattttacatatgctccatgttctaatagtcttaataacaatttaaaaaatacaaatactatgaataatgaaacacgatcgaatatcatttcaaattataaacaggtcaaatattcaaatataagtgtaaataatacacctaataatgtaattatgcaaactcaaaatatgacagtacaaaataatgcaaaaatgtccttaaacggttcgactacacacccaatagtaaatcgaacaaatatggaaaatatagatcttataaaattagctaatgatccattaaacattcatagaaacggtatacatatgggtaacataaagaatgataatatagaagaaaagaaaaatgtatcatcaaatataagaagtatagcagataattctcaacgattacaaaatatacatttaggaaatcaacaaaatgatatacaacatatgaatttggataacataaataataataataataataataatttgaagatgaagaaaaagagtaaaaataacattaatataaataataaccctaataataatcttaataataatggacaaattgataatgcctatttaaatgtatcaccaaccatttcaaattcattacaacaaacaaatatgtaccaaagtgatatgaaaaatagtattatatataatagtagtatacaagaaaaagatccaaataatagaaattataacacacaaggaaataatgtgcaaactatgaatcaatcaaatggtaacgtatacaatgtaaatgtaccaaataggaaacaatccaatttaaatcaatatgtcaatcctatgaatttcttaatacaagttttaaataaatgtaatagtaatacgaatggtcaagttaaagcaagtgcaagtaatgctaatgtgaattatgcaaatgtaaatagtacttccttgaaccagccaaatataaacaatgttggtatgaatcagccaaatataaacaatgttggtatgaatcagccaaatataaacaatgttggtatgaatcagccaaatataaacaatgttggtatgaatcagccaaatataaacaacgttagtatgaatcagccaaatataaacaacgttaatatgaaccagccaaatataaacaacgttagtatgaaccagccaaatataaacaattttagtatgaaccagtcaaatacaaacaattttaatatgaacaagccaagtacaagcagttttagtatgaaccagccaagtacaaacaattttagtatgaacaagtcaagtacaaacaattttagtataaaccagacaagtgcaaacaattttagtatgcatcagccgagtacaaacaattttagtatgaacaaaccaagtacaaacaattttagtacgaacaaaccaagtacaaacaattttagtatgaaccagccaaatataaataatattagtatgaaccaacccaatacaaataatgttcatatgaataatcttaatatgaaccaatctggtatgaatcatacaaatatgacaagtccaatatttaatcaatcaaattcttctaaacaaagcgtcaacgaaaatgcttttgtaccccctgcatatcatacgtcaaatataattcaaaagaatgcaacctcagtatataatgataattttaataatcctaatgtggtgaatgtgggattaaagaatactatgacaaaaaatattatacaaagagtacctcaaaaaaaaggatattctactcatgatatgaaccaacaggagagattacaacaagaaaggttgcaacaagaaagattacaacaagaaagattacagcaagaaagattacaacaagaaagattacaacaagaaaggttgcaacaagaaagattacaacaagaaagattacaacaagaaagattacaacaagaaaggttgcaacaagaaagattacagcaaaaatgggaacaacaaaaaatacaacaagaattatatcaaaaatcacataatgataaggaaagtgaaactaataattcttcatcttatcaagagttaaataatcacactttacaagaaaatacaattgcaaaggattcaatgagacaacacatattcttaaaaacaaggcaacaagaaaatataccaaaagagttgcaaaaaatgcattctcatcaattacaaacagaaaaaatgaaaacccagcaattatctgcacattctttacaacaacaaatgtatccgcagcaaatgacatcacaggtacattcatcctttcacgcgcaaaaaattaaacagcagttacaacaacaaatgaatcatcaacaaataaataaacaccaaatgaatcagcagcaaatgaataagcaacaaatgaatcaacaacaaataaatcaacaacaaatgaatcaacaacaaataaatcaacaacaaataaaccaacaacaaataaaccaacaacaaataaatcaacaacaaataaatcaacaacaaataaaccaacaacaaataaatcaacaacaaataaatcaacaacaaataaaccaacaacaaataaatcaacaatcaatgagtaaatatccatatcaagcacaagaaataaaaaagcatatggatcaactacaacaaaatgtatcaatagtgacacaacaaaaaatgcagccaatttattctgtgaaacaaaacatgccatttaatgaggaaaatgaaatgaaaagaaacacaaggaaaataactttaccaatgactcaccaaattcccaatgtacctaatatacaacaacaaataaataataaaatggatgaaaggatgtcaacagaaaatataaatgagaataacttatataatatgatgaatagaaaattatctacctctataaataaatttaacaatcatgatcagtattacccatatgattcttatcaacaacaacaattacaagggaataatattaatgttatggatattagaaatgttgataaaaatgaaaataatatatttctattaaaaaataatcaatcaagagatattcaaaacaactgtacgaatattatgatgaataataatataccatcatcatatataactccaggacaagcttataaactaaattctaatcaaaatataaatcacacccaacaaaaaaaataatactacaagtgtcatttttacgtataaataaatatcttatttaaaaaaaagaaaaagaaaaagtaacatttttttaaattattaactatatctttctaaatataaaacacttgtatgtttaatatgtgttagacatgaaaaaaaaacaaaaaaaaaaaattaaatgaaaataaaaaaatgtaattttaaaatgctacctctgtagaacaattttgagtacgcatatatttaagaaaaatttataattatgtacatttttttaattaatatattaaagttttattgtccaaatatattttttgagtttttcgtcattcatgtatgtgtttgttattcgtaaacaaaagtttgtgctttataaaatatggaatgacaagtttttttttttttttttttttttatacattattatttaaaaatatttttattatattattatacataatatatatatatatatattttaaattataatattttcatttatgattttttattgattcttataaataattattatagtaattatttttatttttttttattatttttttttttggtttttttaaaatgaaaatttttttttgatgataaaaaacaacacgcctacgtttttgtttttaaaaacattataaatttataatattattttatatatatttgtgcggttaaggattatgtggatatatatatatatatatatatatataatattatactaatatacactttcttcttattaatataagaacagaagataaagctgaaaataccataagtttttttttttttcttttttttttttgataattctattatatatatagaaagaacagaaagaaataataatatatacatattgtgtacatatttctataaaaatataaaatacatgaaaacgtccatttttttatttttttatttttttattaaatatattaaagatattattaatactctttatatatatttttttaatttaatatttaaaatatattttgaatgtatgaatgaataataagcgattcccatataatttccatagaac

SEQ ID NO: 10 - DNA, 2647 bp, Plasmodium falciparum
attaaaattttttaaatattatgaggggtggaggtttttataaaggcacgagtactgaacaaacaccttattttggtgataaggaaaaaaaattgatagaaaaaatagtatggccagaaatatataataagaaaatagatgtaaataaaattaaattcccattaatagaaacatggataaataaaaggttgattgaaattttaggttttgaggatgacatattatatgagtattgtatatcacaattgaagcaaagtaaagagaaaaaaggttaggccgtcttaaatttttttttttttttttttttttactctccttgtacaaatgtatcacaatgtatatatgaatatatatgtaggatatattttatacaatatttatttatttgttcatttatttgttcatttatttgttcatttatttgttcgtttatttgttcattttttttttttttttttttttgagcagatggtgaagaagataaatatctgaatgcaaaaaaactaaaaataaatttaactggtttcataggtaataagaaaagtgatatatttattgaggaattattagaactcttaataaatgaagaaaaaaaggaagaacatattgccgataccttgaatgaaaataaaacaaatgatataaaaaaagtgaaaaatgaaaatgaaaacataaatgagaatgtgtataatggtaagggaaaatatatttaatatgtacataaaatgaattatacatatattaaataaattgtacacatatatatatatatatatatatatatatttatttatttatttatatttatttatttatttatatttatttatatttatttattttgttttattttagaaaataaagatatcagtaataaggacaaagaacatgtcagccatcagaatgaacacaatattaataatgtaaatcttaaaaaagagaaagaatatacagatatacaacgtgataaaagaaaacataaaagatctttatcacaaaaaagtgattcttataaaaagagaccatttaataaaagaaagacttctatcgaaaggtctttatcaaataaaagatacgatgaaaaaactaacaaacgttttaataaaaatttcagaacagacatatataatagaaaacattctaaagatattggatataaacataataattcatatagctcaagtgatgataaatatcataatggtcgttcattatccagtagtgatatatcatatcaacatcgaagaaaagtaaaaaaaaaaaaaataaataaaagacacgatgattcgtcatctgaaataagtagtcatacttatgctagtaggtatgataaagagaggaaaagatttagaaaaagagacataaatttagagagacataaagaaagaggatatataaatagagataaagaaggaaaacatataaggagggataaagaaggaaaacatataaggagggataaagaagaaaaacatataaggagggataaagaaggaaaacatataaggggggaacaagaaagaagacatcaaattaggaataaggaaaagtatagagataggaatgatcataagaaaccatattatgatgaaaacaaatctgaccgttcaagaaatagtagtgataataatgagaaaatgagaaaaaaaggaaaaaataaaaaacacatgaaacgttatacgtcatcgaaatcggttagtaaaagtagcaacacgtcaattaatagatcaagtgataggtcaagagacagatcaagtgataggtcaagtgacaaatcaagtgataggtcaagagacagatcaagtgatagatcaagtgatagatcaagtgacagatcaagtaacagatcaagtaatcgatcaagtgatcgatcaagtgacagatcaattaataggtcaagtgataaatcaattaatagatcgagtgatagatcaagtagtagatcaaataatccaagtgattcgtttaataataaatctgaatcttcagaaaaaattaagcgagataaaagagaaaaaataataaattctgatagaaataaaagaaacaggtaatagctacatctttatttttatttatttattcatttttacttcaaatcttccctcttaaaatgtgtgtgtccaattttttttttttttttttttttttttttttttatgattattcctttatctttctattatcagttctataacggtgtctagagatataaaaagaaaagaaatagataatataatgcgaaaaaaaagttacagaacttcaaaaaaaaaaatgagggcatggtcatcgtcagaaagtagcgactctggagaaaagtaaaaaaaaaattatatatataatgaaatatatatatatatatatatatatatatatatagaattataaaaagaaaaaaattcacctaattaaacacttgcattgaatgtatattataaaatgcggaaaaccattttcatttcttttatttatttaaaacaaaatatagacaccacatttttagagtatccacttttttttttatttgaataatattttcatttcgaaaaggaaaattaatatttaatagttttgtctaattccataaaaaggatatatttatcaagttatatatatatatatatatataatttattttatgtgagag

SEQ ID NO: 11 - DNA, 1902 bp, Plasmodium falciparum
ttttttttgtttcaattatataaatttatatttttaaacattttaacgaattgatgaaatgtaaaaaaataaagaaaaaaaacatcattatagaaaataagttggtatttaaaaaataatatatatattaaatatatatatatatatatatatatatatatatatatgtgcttataattaagtttatttaaaattcagttgtttatttatttatttatttatttatttttttttttttttaacgttcacctttcatatggaaaatatagagaaggaaaacaaggaagaaaattatttggacagacttacaaatttgtttaaatatttaacattaggtataagatagaaaagttttttttttaaaataaatatatacaagaacaattttttaatatgtgtatatatttatatataacctatttttttttttttttttttttttatattgagataaaaaggtgtagaaaaaaataatagtacagaaaaaaattcgactcattctattaaatatgatggtcataaaaatagtttacaacaagttgtttataatttaaaattgaagagggcatctgagttaagcgttcctcacagaattttgggaaacacgagcatatccaagaatggttacacatatatatatatatatatatatatatatattttttttttttttgtgagacattgtttttatttatttttctttttatcatagattctgataaagaaagagataacaatacaaagaaggaagaaaatgatgatacacgtgatagtcaaatatatgatcacaacatatgtggacttattaaagaacaccaagatattcaaacaatatctttacaacaagaagatgacaaaaaggtttatttttactcttctagtatagatcttgaacatggggaaatttataaaatagaccaagcacataagaaaaaaaagaaaaaaaaaaaaaaaaaaaaggaaaaggaaaaggaaaaggaaaaggaaaaggaaaaggaaaaggtaaaggaaaaggaaaaggaaaaggaaaaggaaaaggaaaaggaaaaggaaaaggaaaaacataaagatgaacataaagatgaacataaagatgaacataaagacacatataatgataaacatagtgataaacatagtgataaacatagtgataaacataatgataaacatagtgataaacatagtgataaacatagtgataaacataatgataaacataatgataaacataatgataaacataataataaacataataacaaatacaaaaaaagaaaaagtaaaagtaaactatatgaattagaagaacaaaattattcaaagaataaaataaaactggttgattcagaaaaagaaaatattctcatgacaaatttaaaaagtgtctcgtcctctaataattcagaagatataaagaatgaaccaaacataaaacaaaatattatcaataataatttatatacattacataaaaataaaacatacataagtataagaagtaataaagaaaaaaatcgtgtaattaaattaccaaaactaaatttcacaaataacctcgaatcttcttcaaatgactaaacgagaggattattaataaaaaaaaaaaagaaaaaaaaaaaaaaaaaagaaaaaaaaaaagtatatttttttgaaaatccattcattataatattccattaaaatttaacaatatatacatatatatatatatacatatatgtgtgtttttttttttttttttccttgttgaataattattcattttaaaatgcatgtctgattttttgttgctcaaaatataattcataatcatatgtattaggaaaaaaaaaaaaaaaaaaaaaaaaatatatatacatatatatatatatatatatatatatgcacaaaaaaaaaaaa

SEQ ID NO: 12 - DNA, 10029 bp, Plasmodium falciparum
atatatgtatatatttttgaagagggaaaaatatggtgttcttacattcttttcatttttatcctttttaaaaatttattaagtaaaccaattgataaatgtgatcacatattttggtgtgtttcactttttttttttatttctaaaaaaaagaaaagaaaaaaagaactttttatatttaattggtacttgttcttttaatagaataagttgtgtgcaaatcttatagaaaaatgaatcaatatgatcaatggaatcgtagaattcgggtaggaaaaaaaaaaaaaaataaagaaaaataaaattaaatatatatactttgttgaattaattatattatattgttttattttattttaattatttattttatttttttttttttttttccttttagacaatatggtactgtagtagtctgtatgtagaatataagaacgctttgaggaaatccaaaaaaatgccggtggagaaaaaaaatgaatactgggaaaagaagcatgaagaatttgctacaaagatgttaaataatatatatgaattaagaggtaaggaatattattataaaagttaaaaaaaaaaaaaaaaaaaaaaaaaataatatatataaatataaatatataatacatgaatgcgttgtctatttttcatattttataaaaaaataccactacacgtttatattatctttatttatttatttgtttgtttgtttattataaataggttggtgggttaaagtaggacaatttttaagtacccaggaaaatatcatgcctgttgcttatatagaaaaatttacgaagctgcaagatatgatgcctacgtcttcatttgataaaatagaaaatattttaaaaaaggaattaggtaatttaaaaaaaaaaataaaaaataaatgtgtctatgtatgcataaaagtatgagtatatgtatgtataaatttatgtacaaatgtatgcgtatatgtatgtataaatttatgtacaaatgtattcgtatatgtatgtataaatttatgtacaaaagtatgcgtatatgtatatataaatatatgtattttttttttttttttgtttgattcctaggtaacatttatgaaatgtttgaatacatcgataaagaaccactagctagcgcttccattggacaagtacataaagcgaaattaaaaaaatatgataaacatatatgcgatatagacagtagaaagcataaggattataatgtaataataaaaattcaacatgaaggaatagatcagtttttgtcatcagatataagtactttaaaaaaagtgtcttgggcatttggattaatagataagaatttttattttacagattttatagatgaatggcaagattctgcttctcgagaattaaattataaatatgaattatatcatcaattgttagcttataacagttttaaaaaatcaggtatccctgtaaaaatacctaaaatttattgtgctcatacaacttctaaggtattggttatggaatatattaaaggctttaaaataactgatactgaattacttaaaaaatataatgtagatacatataaaatggtctataaaattattgactattttgcttatcaaatacataatgatggtttttttcatggggacccacatccgggaaatatattagtcatgatggaagaaaagaaaaaaagaaaaaggaaaaaaaaatataataaagataaaataaataactcgaaaatgaaaatgaaaaatgaaaatatatatatgaacaagagaaaaaaaaatcataaaaaaagtaataggaattctaagaaaaagttaagaagttgtaaaaaaaaaaaactagaaataaataaagaaagagaaaaaggagaagaaaagaaaaaaaatatgtataataataatgatataaattattgtgaaaataaaaatgaagatattataacaaacaatcccaaattacaaaataatagtagtgatatgaaaaatataataaaatttacaagttattccaaaagtgtatcttcatctaatgcttcattgatggatcattttgtggattcaagatataatattgataaaatgagagctcctaataatttttcgaatccttttcataaatcttttagttttgttgattattcacttaatcaagatgtagattccgataaaatgaatcaagaagattatgaatatattttgaaggaaaatatgagtgatggtttagataatatgaaaaattgtctctcaatatcccatgaatatatttcatgcgatcaatcaagaagtggagtaacatctattgcgacaagtaggaaaacacaaaatagggattctaacaaagctagtgaaaatagtattatggaatctcgagaaacaacgatgttttcttttgttaacaatatggaggagttagtcaagatgaaatccgagactaaaacggatctacaaattaaaggtaatgataaaaaagtggttaaaggtagtaagtgtgacaataaaaatgatcaaaatgttaataatgataatattaataatgataatattaataatgataaaattaataatgataatattaataatgataatattaataataataatattgttaataataataacaatatttctaatggtgagaaagagaaaaacaaaagtggtccgatatacaaatccaaattcaaactatcagatttaaaaaaaagaaaaaatgcaaaatataattttgtacctgttattattgattggggtttaattaaacaattagatagcgtaatgaaattcgccttttgtaaattagtttataatattagttgtatgaatgtattaaatattatagaagcctttgaagatatgggtttctgctttaaagaagattttacatacgatcctgaaatatatattgagaatttgaaaacatactttttaagcaaattagaagaatctaaaagtaaaggtaatgaaggagaaaaggaatttggtagtgctaatacaattgatgaagcaaataagagtaataagaatatggaaatattaaaaaatattgaaaaaaaagatgttatggataaaaatccgataagtgatgtacctaaagatattatattttttatgagagtagcatcattattacatggattatgtacacaattaaatgtaaaaattaattatttaaatattttttccagaagagctaaagaagctttagaaaaaatatatataccaataaataatagtatacacacaataccaatagataaatgtccaaatacatttgtagaaaaaagaattcatcaatttataaaaacattatatgaaaaaaaaaaaatattaggatgtcaagtagccattatacataaaaaaaagctagttgttaatacatgtgtaggaattacaagtacaacagataaaaggcctataacaaaacattctttatttaatggatattcattaaacaaaattattttaaatatagctctatatcatcttatatgtaactcagtaaatgaacaaacgtcaggtgaaagtattctgctcggtatggataagaaaaaggataagctaggggaaaagataagtcaaaaaattcaagaaaaaataaatgaaaagataaaaattaaatcaagtgcgaaggacaaaaatcaatccgatgaaaaaattaaaatgaagataaacgaaaaggataaggataaggataagggtaaaggtaaggaaaagaataaaaataataataagcaaaacaatgatagggataaaaataataatgaggaaaacaataataatgaggacaataataaggataacaataataataataataataataataataataataataataataataataataataaattattaaacgaaatggatcaaagtaaacacaatgcaaatgaaaaaacatccaatatgaaaaataacaataaactttatgaagaagaagatgtagaggatgaagttgctgatgataataaaacagataatgaaaataattatattaccgaaaaattaaatgaatttaataaaatagaaaacgattatttttcagcaaatgttaatgaaaatataattaaatgtgaagagtttggtgagagcaaggatattataagtcctttatctgtcaatagtttcataagttttgatagttataatagttataataataatgttagttggatcagtagtatgagtaggaatagttacaaaagtatgattgaaaggatagaagatgatgtggaatataaagaaattaatgatgatctacctaatgatagtaaggttagttgtatgagcgaatacaaaaaggatgataaatataaagggagaaattatatacatgatcacaataatgatgatgtacatcatcataataatcataataatcataataatcacaatcatcaccataatcatcaccataatcatcaccataatcatcaccataatcatcaccataatcatcaccataatcatcaccataatcatcaccaccatcaccatcatgataataatataaataaaagtaataaagatacagagtccaatccaaataaatccataaatatatatccaaatatatcatataacaaaatggataccttaatacaaaaaaaacaaagttatattgatgagaaaaactttataaaaagtatagaaagcaaacaaataagaaatttcaaaaatattattaacgatcatatttgtaattactgggatggatttatatgtaataataaaaaaaatataacgataaaagatatattaactttaaagtgttttattaagaaaccttttcatgataaaataacgttaagcaaatttatagattacgatcaaatgataaatatgattgagaactctaagcattataagagttcagataagacgtcgaaatatggagagtatttatatttaatcgatacgtatattatatctgagttaatacataatatatcaggattacattattatgagtatatatataaatatatagtgaaaccattaaatttgactgaagaaatgtttgtacctataccttcagaattattaaaaaatcaaaaatacgaagattcaaaaaagaaaaatgtcaacaatcaaaataatgtaaataccactggtggtcttagtaataataatttagataaaacaaaaaaatatataaaggataaaaagacaaatgttatagataagaaatctattgttatggataaatggcctaacatgatgaaggattatacaaccggtgctttaaaaaataatgaatacaagtcaacttcatttattggtggtaagaaatttaaaaagtctagtactatttccaactttatagacgatataaatgatgatatgaatttttatattatgaacgcggaacttttaaagtttgaaaatattaatttaaagaataccatatctaacaaaaaaaaatttgatagtaatcaaaaagttttatccaaaaaaagaagtataagtgtagatgtgtattattctaagaaaaaaattaaaggtaacacaataaatagaataaaataagaacaagtgaatagataaaaaaaaaaaaataaaaaaaaaaataaacacatcaaaattgatatataaagaaaaaatatattcataaagatgtaatatgtaatatgtaatatgtaatatatatatatatatatatatatatatatatatttatttatttatttattttttgtttatttatagatggtagtaagaaggagaaaaaaaaaaataaggataagaaagatcaaaagggaagtaaaaccaaaatggattttgaaaaaagagacattgcagaaatgaagaaagtagataggaatgaacagaaagaccaaacgaatcaacagaaagatcaaacgaatgaacagaaaaaccaaccgagtcaacaaaacgaacaaatgtatgatgacatagttgaaacgtatgaagatataattgaaacatatgaagacaaaatagaactaaaccatgataatgatgatagtaacataaaaacgaatgatgtttatcatgacattatagaagagaaagaagacaatgaatattataataatattaagaatgaatatgagaataaaaggaataataataaaattgaaatatcaaaggattctttaaataagaatccatctaatatagaatgtaaaatgaatattttaataaataacgaacttatagataatgaaaatgcaagaaaattagattcaagtaatcttcttgaaagtaccttaaggaataaagaatcctttagttttaaaaataaatttctgaatcatgtagataatttaaaattaaaaacgaagaaaataaatgattctataaaaaatatgtatgaaaataatgagaatatttttttgaaaaatttctttctatcatctaagaaatatgataatatagaaaataacttagatcacagaaatgtagaggctaattgtgtagataataatatgtgtgaaagtaatagtctgaataatagttgttataataatagtttttataatagtttttataatagtcatagtaatagtaaaaatagtagtaaaataagaaactacaataatatggataaggaaaataataatgttaatagtgataatataaatttcaatcccaatgataataaaaagaaaaatagttataataataataataataataataataacaatattatggtgagttctagcccttatatttataattacaacaataaatatatattaaaagaaaattatgataaaaaatttaatatgaacaaatttaataaagtaatgttatctaatcaaaaagaaccagttcataaaaataataaaaaagataatttatttttatttgaagaaagaggtaagagatcacatagttatagtgactgtagaaatgaatcacaccatgtaatgaaaaattcttatatggagaatattaacaatataaataattatagtaatagtagtatgaattacttaaactattatattagtaataagaaaagtgtaaattatatgaatgctaatggggtcgtcataaattatgatgataataataataatgtaaataatgttaataatgtaaataatatgaataatataaataacaatgaaactaataatactaacaaaatgagcttctttacaaacactatcttgaatacttttcaagataaaaataataagacaaaaaagaaaaaagaagtgaaatgtttaagtaatgaagataaatatttttataatttagaagaaagaagaaaatattataaatataattttgataaaaatatcagaaagttatttaacaatgtggatacaaaaacgagtggtgaagaatcagcaaatagtctattgaaagaaaaatataataaatatcataagagaaataaaaaatataaaaaattgttcgaatatatcaaacaattaaaagatcatataaaaaaaaaaagcgatcatattagagaggcaaatgaagcatttataagaagtataaaacaaaagcaagaggacgaaaataaaaaaaaagaagagaataaaaaaaaagaagagaataaaacaactgatgggggtaaaacaactgatgggggtaaaacaactgatgagggtaaaataattgaagagggtaaaataattgaagagggtaaaacaactgatgagggtaaaacaactgatgagggtaaaacaattgatgagtgtaaaacaactgatgagtgtaaaacaactgatgagtgtaaaacaactgatgagtgtaaaacaactgatgaaggtaaaacaattgaagagtgcaaaatcattgaagataataaaaagggtagtgaagattacaataaaaatgaagagaataaatcgaaagaggaaaaaacaccaaacgatgacaaatacgaatttgttcaagatactgaagagctcataaaaaaggttacgaaaaaagatcaagagttgttgaaaaaattaatagaatataaataccaaatattaaaaaaacaaaataataaccttatgaaaaaaaaaaataaagaaaatatatatatgaacgattattcagataatatagagatgtttagtgatactgaaacggattctactgattattatgatgaagatgatgcagattatgatgaacatatggttgaatataataattatcatgatgaaagtaatcagaatacaaaagattatatattttatgataagaaaaaaacgagaagagaactaaaagaattacgacgttttaatgaaaggaagaaaaggttagaaacatatttaattatgaataagttatataatcaaaataaaaaaggagtgatatcaaaagaatatcatacaattaataaaaaaacggatgtatattggcgactagcatttgcaagaagaaatatcaatgtattagaaaacgtaacacaagaggatgtgaataatcaattagaaatgttaaagtcaaatgggaaggtacaaaaaaagggagcagcacataataaggaaaatgaaaatgaggaaaaaaataaaaataataaaaataattatattcataatagtagtaataatataaagaatgatcaagagaaatcatcactaaatgataataaaaattttgataacaaacttaaaatgtatgtagataattttaatattaaacaatataataaatatatatcattattccaattaatgcaatcgaaaccatatattttagatccattaatttatgattcaaaaaaaattttggataaatttataccaatcaatggtagatttacagcaaaagcattatgtaaattatttgcatttgtgaataacgaatttttttttccatcttatattattaataaaataagaaagaaatatactattgataaaagtgtagaggcattgattttaacaggagctatgagtagaacatggggtatgggttttcaactttttgaatgtgaatataatgctaatattaatgatgaatttaatttatataaaaaaaaaaataaatataaaaaaaaaaatgtagaatctagaaaaaaaaaaaaaataaataaaaaaaaaaaaaaaattgttggatatggacaatccgatttttcaggatgtttggctatatcatttccagaaattgatttatctatcacaatacttttatctgacgtttttaaaggagcagacgtaatagaaaatgaaaataattcaacaattatgtgttatatatgatatatgttaagtctcaatatttatttaatttatttaatttatttaatttatttaatttattcattttttttttttttttttttttctttcttttctatcgttttcttaggtgtgtcatctcttattagaatttatattaaaactatacggattgaaacctcaatggaaagtgcctgtcaaaatgtctgaacttgtcaaagttttttaaaattaaaaaaaatgacagaaaaaagaaagcatcttttttgtcgtcctacaattcgtaatacacacacatatatatatatattatatatatgtgtttatttcttcctaatttttttagttgtacttttttttttttttattattatatatatttttttttacccagtagacacaggaatatactcttaataattcccctttattttttaatttgtcaaattatacaaatttatttttaacatagaattttataaaattattatactagaaagagatttattttattttattttatttttttatttatatttatttatttattttttatttccgacaaaaagaaatatatggctaaattaaatataaatagcgtaaatgttatgttgcatatataaatcattaagataataatttatttgattcatatactatatatatgtgttatttaatatgaaacagttttttttttttaattttaatatttttcgttgttttattttatttgtttttatttcctattttctgtttttttttttttttttttttattccttgcgtttaaaatgtttttataataaatacataaataaaattcatattaataatttaaataataaatatatatatatatatattt

SEQ ID NO: 13 - DNA, 4140 bp, Plasmodium falciparum
atgaatcacgttgatgattatgattacttctgtcatagttgtgaagatgtaaaaagaactagtgatgttgaaattatttacaatggtgagatcaaatgtaagggttgtaacaacgtagggtttgttgaaaaaattgatgaagatgaccctttgagttttcattcattacatactagtagggatgtaggagaaagtcgaagtgggccaaatagcagaaatgataataataataatgatggtaacaataatagtaataatacaaatagtagacattataattatagaaatatgaacaattttggttatcgtaccaatataccaagaagtcgaagaaataaccataacaataataataataatgatgatggtagtaataacaatcgtcacaatagtagtaatagtaataataataataataatactagaagtagtcatgtacgaggagaacatgataatttttttaacgattttatgttatttagaaatatgtaccaacaaatatttaatccttcctttcgagctgcagatgctgaagtacattttacagcggcggtaggttcctcaaacaattctaatgatattaatagacataataataatagaaataataccaccacacccaccaccaacaacaataataataataataataataataataataacaataacaacaacaataataacaataataaccatatgggtaatagaaattctagtcgtaataatgagaatacgaacgaaactaatgaaaatgagactagccaaaatgataatagaaatagaaatatatttggaaggacctatagttataataggaatacaagaaatccacgaataataacaagagctatcgatataactaatataccatttaattctccaggacgttttaatttccatgatttgatagataacatgtttgcgaatacatttgataatatatccttagatcaagtgttaacaataattatggagagtgacccatctcgaaacggtcctccgccagcttctgaagaagtaattaagaatttgaaagtagaaacattaacagaagagagagcaaaggaattagaatcatgtgcaatatgtagagaggaatataaagaaaatgatgaagtgcatagaataacagataatgagcgatgtagacatgtctttcattgttcttgtattataccttggttaaaggaaagaaactcatgtcctacatgtagatttgagttacctacggatgatcaggagtataattgtaagagggaggagttgagggaaagattgaattctgagatgtctcgaaataatacctacaatggtactgacaatgataataataataataataatgataataataatgataataataataatgataataataatgataataataataatgataataataatgataataataatgataataataataataacagcaataatgatagtaataacaacaataatgatagtaataacaacaataatgatagtaataacaacaataatgatagtaataacaacaataatgatagtaataacaacaacaatgataataacagcaataataataatagtcgtagacataatgccgatgaagcgaattataataccttaggagatgactacaatattaacatattagactcttttacaaatgaacaaacgacttcaatattacaacaagtttttaacagttttttatcgggaagcaacgattcctatagaatagacagaatctatttatcaccagataattattttagttacagtagtaatagaaatagaaacaatataaatgatgagaatgtggaagtaattcaagttgaagagacccgttccgattataatgaatcacatgacgaaacaaatgaaaataatgaaaaggaagaaataaatagggaggaaggaaatgaagaagagaaaagaagtaagaggaagaaaacgaatgattatgatgataataaggatgtttataataatgcaagtggtagtaatagtaatcatagtaatcatagtaataataataataataataaagggaatgaaaaaaatgagcgtaaggaagatatgtgtaatgtatcaagtggtagcaataataatagaaattcatcttctagatcaaattattataacaatagatatcgttataataattatcgacgtagcaatagacgtaatatggatcaagattattttaatatacaagatgatgaagtagaggaagaaaattattattattgtacccctccaaacgaaacaattaaaaataatacaagaggacaaagaaataatgacacagataatgtaagaaataacaacaataatgataatgataataaaaataatgataataaaaataacgataatgataacaacaataataataatacaagacgtagaaataattattatgataataattcatataataatccatcttatttacatgaaaatgtttttgatacatctaatgtgtatgtaataagtgaaagaacccattcgaatgatagaagcaacgatagtaatacgagaactatatcacagctttatttaacagatttaagtgatgtttttgctaattttaattatagttgtaattttaataatgatcaatatattataaataatgaagaggatcatacgaaggatcatgatggaaaagatgaacaagaaggagaaaaagaaaaaacaaagaatgtaaattctgatcatatggaatccaatataaatgattcaggagtctctttaggtggaccaaaagaaaacaaatccaatgataagaatgaacatcaagtaaaagagaaaagggcatccttcgaaataaatactggtaagaccagtacaacgaaaaagaatgaaaagagattatatgaaaaggttaataatactacatatagtaatgcgagtaatagtaacaatagtacaaggagaagtagagatagtgataatgagaaagagggaagtagaaaaaaatcaaagaattataaaaatgaagaggaatacagacaaagcattaataataacaataataataataataataataataatgaagatagaagaaatagtaacacaagctgtagccataaatataataacaatacagacaatgctcctaatcgcaatccttccgatgtggttaataataatgaatcaaacgaagaacagaggaatactaccaaaagacaaagtactgtgaagaaataccttttcgataagttgaattggttaaaaagttatgataataaagataaaaaaaaaaatgaaaaaaatcaagattcaaataataaaagtgatgaaaatgatacggaacataatcaaaatgatgataatgccacaggtatgtcgaacgaaaataatgacgatcatacatcacatttgtaaaaaaaaaaaaaataaaaaataaaaagacaaacttaaagtagtcaattatatatatgtatcaaccaagttgataatgacagaacgatagatatgtagacacaaaaaaaaatatatatatatatatatacttatatatgtatgtttttttttttttctttttttttttgttaaaagagaaatgtttctcaaaaaaataaaatacactgaaagttataattgtatatatatatatatatatatatatatatgtccgttacaaccatatcagatgtttccatggacctataatcagatcatttgttttttttttttttttttttatttgaaaaatgagccaacaaaagtaatatatatatgtatataatgatgggaatataacatatttttttgttaatatattagagtaatactaaagatgaaccacataaagaacgcatatttattatatatgtatatatttgtgggaaaaatgttataagaattctttaaaaagaaaaaaaaaaaaaaaaaaaaaaaaaaaaaagtgctatttaagtattttattattaatatattttttttttttttttttttttttttgtttgttaattaaaatatattagtgattttaaatatgtatgcatcataatatattatataaatatatattgtttcttttttttatattgataaaaattataacaaataatatatatatatatatatgtatatattaattagttttttttattattatttatgatttcc

SEQ ID NO: 14 - protein, 141 aa, Plasmodium falciparum
MKKGNHLHINRIQNLIREKEMTLLEKSIVWGKIISKRKNSQLLIRKRREHWILLKKREKVLIFMRTRKKYQILMRKRKKYQILMRKGKKYQILMRKGKKYQILMRKGKKYQILMRKGKKYQICMRKKRRVLQNLMRKMMKY

SEQ ID NO: 15 - protein, 187 aa, Plasmodium falciparum
MDAHKLINKLINKLINILINILINIYIYKINKINKLINKLINIYIYKINKYINKFINKLINILIHRHIIYIFLFIHLHIIFPNGDDISSLPIILYSGLILIIYNSFFPLYFYKLYVYIYIYISFALLILINLFLGCFGWSTPPILSPTDICWFGFTFHLKFFLFSKITIMDEPTLNSPTSSPISRSL

SEQ ID NO: 16 - protein, 23 aa, Plasmodium falciparum
MKSWEIIVKITKQMKKVIEKVKD

SEQ ID NO: 17 - protein, 90 aa, Plasmodium falciparum
MYKYTFFIDEHIIILKNVIIFFKHFIFISFFLFFFTKNINILQKKKKKKKKIHINKIYIYIYIYIYIYMCVFFYRNKNKDKICCRTYSYT

SEQ ID NO: 18 - protein, 49 aa, Plasmodium falciparum
MWLQLFIYIFIYIFIYFSYNHKHNFFHIEDILVFLKYWNILVHLMMNNQ

SEQ ID NO: 19 - protein, 68 aa, Plasmodium falciparum
MIIMKIIYLINGMVKYIVPRKKKQQKNLMKKMNLIQMLNKIFLPIKVTKEIKKELMMIITWIMKTIYI

SEQ ID NO: 20 - protein, 23 aa, Plasmodium falciparum
MYKIQIIKKKLIQQMKIKKKYFS

SEQ ID NO: 21 - protein, 78 aa, Plasmodium falciparum
MYNIIYLSLYIYITWYIKQKKNKKKKLYLWIIKKLNQNIRTTFQFKKIIIINKMHTTYILYKHKNELPTSFSLNNTLN

SEQ ID NO: 22 - protein, 72 aa, Plasmodium falciparum
MIWTNRRDYNKKGCNKKDYNKKDYNKKDYNKKGCNKKDYSKNGNNKKYNKNYIKNHIMIRKVKLIILHLIKS

SEQ ID NO: 23 - protein, 152 aa, Plasmodium falciparum
MIIRNHIMMKTNLTVQEIVVIIMRKWEKKEKIKNTWNVIRHRNRLVKVATRQLIDQVIGQETDQVIGQVTNQVIGQETDQVIDQVIDQVTDQVTDQVIDQVIDQVTDQLIGQVINQLIDRVIDQVVDQIIQVIRLIINLNLQKKLSEIKEKK

SEQ ID NO: 24 - protein, 21 aa, Plasmodium falciparum
MKCKKIKKKNIIIENKLVFKK

SEQ ID NO: 25 - protein, 39 aa, Plasmodium falciparum
MWIQKRVVKNQQIVYWKKNIINIIREIKNIKNCSNISNN

SEQ ID NO: 26 - protein, 120 aa, Plasmodium falciparum
MIRSIIVRGRSWGKDWILRCLEIIPTMVLTMIIIIIIMIIIMIIIIMIIIMIIIMIIIIITAIMIVITTIMIVITTIMIVITTIMIVITTIMIVITTTMIITAIIIIVVDIMPMKRIIIP

SEQ ID NO: 27 - protein, 30 aa, Plasmodium falciparum
MLCIHTLYLFKKNPQNDMFYYLRNNLLMLK

SEQ ID NO: 28 - protein, 868 aa, Plasmodium falciparum
MNKENIEKLINIINLLDKISENKRDKNNYGEHNVNKFLSSELSKTLDSLEYRKSLFPLCDEKEKVNSNVNKIISNFKTTKSFFYFNKSSSLNMGEDMDENMDENMGENMDENMGENMDENMGENMDENMGENIDKNIDKNIDTNMDENIYSNKHLRKNIYLLSSNVKSSHIKKDNKNIMKYTNNVNINDNDIYLNTNQKNVKYNYLNVIQLNEKKYKRENSFSSSSTIFSFIKLKTDNVIEQRNTSNVLRNYFSDTELLSYNNNHKSKYNLLKMLKRDELKKYEQEKFKVQNKGRQNQVEKEELMEELEDGCIDNNTTGLLLEKIIEHLNINLKHILNNYKKEINKLCKLNDTLSGKLENALENNDEQAQEIRDLNKKIHIIKEQKTEMNKIIEDYEFQTVNRKKLENSQQNYIKERDKLDDEIKYLKNELNMMKSTNLKIKEEKLLLKDKFKKYKGKYHNQKEKLQNANELILELKLAQNEQITEKNLNIINNEYSKQRDIFPYEKKRKIIINRFRSDTIMDNFERMKKKLFESNERCYILSKTNLQLFKGIRNRKKKIYTMNREMSNLKHILLKGKEGMSRCTLLDLKENTAGLGTSNSNVSYNGLCKRCCKLKDENDTISEKSYNNKICNNIIDEKMLYENSSNFDLILNRKNNEYNLLKDRYDVLYKKYIKLLTTYSCKNEDFTFIKTKTQEEMIPKKDVFHSFYVKNTKNNIEKCHSKDNKLISYIKAKVVNKKDICKKKFKHYTESNNYRDLEFYISNQILNKLDMKDILNIRYVYSYGNKNGKILCHNTNSLDNNTCIYLNIYIYIYIFIYCYILVSNKFYHVSLYIKTKIHIRKKKKKKQINKYLKNNINLFNIIIIYYY